(19) United States
(12) Patent Application Publication
(10) Pub. No.: US 2026/0162792 A1
(43) Pub. Date: Jun. 11, 2026

(54) SYSTEMS AND METHODS FOR CLINICAL GUIDANCE PROTOCOL GENERATION

(71) Applicant: ZOLL MEDICAL CORPORATION, Chelmsford, MA (US)

(72) Inventors: Paolo Giacometti, North Grafton, MA (US); George Beck, Salem, MA (US); Guy R. Johnson, Wilton, NH (US); David M. Betz, Bedford, NH (US); Lisa M. Campana, Waltham, MA (US)

(21) Appl. No.: 19/181,758

(22) Filed: Apr. 17, 2025

Related U.S. Application Data
(63) Continuation of application No. PCT/US2023/077193, filed on Oct. 18, 2023.
(60) Provisional application No. 63/380,131, filed on Oct. 19, 2022.

(51) Int. Cl. G16H 20/00; G16H 40/20; G16H 50/20; G16H 50/30; G16H 70/20

(52) U.S. Cl. CPC G16H 20/00; G16H 40/20; G16H 50/20; G16H 50/30; G16H 70/20

(57) ABSTRACT

An example of a system for generating encoded clinical guidance protocols includes a clinical guidance protocol generation (CGPG) GUI and a clinical guidance protocol platform including a CGPG engine configured to receive a user selection at the CGPG GUI of protocol blocks including at least one unspecified protocol block, receive a user specification at the CGPG GUI for the unspecified protocol block, convert the unspecified protocol block to a specified protocol block based on the user specification, receive user indications at the CGPG GUI of sequencing links between the plurality of protocol blocks, connect the plurality of protocol blocks according to the user-indicated sequencing links, generate an encoded clinical guidance protocol including the protocol blocks connected by the sequencing links, and export the encoded protocol to a clinical guidance engine configured to implement at least a portion of the encoded protocol at a clinical guidance user interface.

### BACKGROUND

In a pre-hospital and/or acute care treatment setting, medical responders are often tasked with providing interventions for emergency conditions such as respiratory distress, cardiac arrest, and/or trauma in a treatment environment that is often noisy, crowded, and chaotic. As an example, the scene of an automobile collision may be at the side of a busy highway during a snowstorm and involve multiple victims. In many cases, medical responders are encountering the patient for the first time without knowledge of medical history and may have to perform immediate interventions to prevent the patient from dying before the patient can be transported to a hospital or another setting providing advanced care and diagnostics. In such settings, even well-trained paramedics, nurses, or physicians often have difficulty in rapidly assessing and recognizing patient conditions and in identifying and providing the most effective immediate interventions. In some cases, although optimized and efficient care provided by first responders is often critical for ensuring a positive patient outcome, in some cases the first responder may have limited training for interventions for a particular medical condition or injury. However, aside from training concerns, in any emergency medical response, a caregiver is often presented with distractions and an overwhelming amount of information that must be synthesized to provide effective care. This is a challenge even for medical practitioners with advanced levels of training such as paramedics or physicians. Given this challenge, it is possible for medical practitioners to miss key decision drivers. This situation becomes particularly acute in the case of medical events involving complex interactions between physiological systems and/or high acuity low incidence events where the medical data driving decisions is sparse and/or conflicting and where the treatment protocols, including, for example, medication dosages and therapeutic targets, are highly specific and infrequently applied. This may challenge a practitioner's ability to recall and apply these protocols.

To alleviate these difficulties, rescuers and caregivers can benefit from tools that guide them in decision-making and in providing interventions. Such tools may automatically monitor, integrate, and analyze a variety of information provided by both caregivers and medical devices. The tools may use this information to provide automated and life-saving guidance for effective and immediate medical care. However, to be effective, such tool should provide this information without distracting caregivers and without creating extraneous administrative or recordation tasks.

### SUMMARY

The foregoing general description of the illustrative implementations and the following detailed description thereof are merely exemplary aspects of the teachings of this disclosure and are not restrictive.

An example of a system for generating encoded clinical guidance protocols includes a clinical guidance protocol generation (CGPG) graphical user interface (GUI), and a clinical guidance protocol platform including a CGPG engine configured to receive a user selection at the CGPG GUI of a plurality of protocol blocks including at least one unspecified protocol block, receive a user specification at the CGPG GUI for the at least one unspecified protocol block, convert the at least one unspecified protocol block to at least one specified protocol block based on the user specification, receive user indications at the CGPG GUI of sequencing links between the plurality of protocol blocks, connect the plurality of protocol blocks according to the user-indicated sequencing links, generate an encoded clinical guidance protocol including the plurality of protocol blocks connected by the user-indicated sequencing links, and export the encoded clinical guidance protocol to a clinical guidance engine configured to implement at least a portion of the encoded clinical guidance protocol at a clinical guidance user interface (UI).

Implementations of such a system may include one or more of the following features.

In general, and in one or more embodiments described herein, the plurality of protocol blocks each represent one or more instructions that, once exported, are configured to provide for control of one or more medical devices, for control of one or more clinical guidance user interfaces, or for control of a combination of one or more medical devices and one or more clinical guidance user interfaces. The one or more medical devices may provide the clinical guidance user interface. In other examples, a computing device may be configured to provide the clinical guidance user interface and may be communicatively coupled with the one or more medical devices. For example, the protocol blocks that form the generated encoded clinical guidance protocol may enable one or more of closed-loop control of one or more medical devices; information requests from the one or more medical devices such as from one or more sensors or electrodes of the one or more medical devices; information storage at the one or more medical devices; determination of operational settings one or more medical devices; determination of inventory of one or more medical devices or other available devices; display guidance and/or feedback to a user of the one or more medical devices; request information from a user of the one or more medical devices via the clinical guidance user interface; and provide clinical guidance tailored to a specific medical device of the one or more medical devices available during patient care. Accordingly, in one or more examples, the system for generating encoded clinical guidance protocols described herein may be configured to enable the generation of different encoded clinical guidance protocols based on the user selection, the user specification and the user indications for the protocol blocks, tailored to an intended use-case for the one or more medical devices and/or one or more clinical guidance user interfaces that are to be controlled by the exported encoded clinical guidance protocol. Further, in one or more examples, the system for generating encoded clinical guidance protocols described herein may be provided in combination with one or more or a plurality of medical devices and/or computing devices configured to receive different exported encoded clinical guidance protocols generated by the system. This may provide the advantage that a medical organization having a fleet of medical devices and/or computing devices may provide and/or update custom protocols to the all or selected portions of the fleet of medical devices and/or computing devices from a central location and thus cause devices to operate according to a uniform care standard as defined by the custom protocols. Alternatively, this may provide the advantage of enabling a centralized control of protocols customized to a particular environment in which the medical device and/or computing device is located. In either case, the solution provided herein alleviates the risk of lack of protocol adherence created when protocols must be loaded or specified at each individual device.

The at least one unspecified protocol block may include at least one unspecified foundation block, the plurality of protocol blocks may include at least one workflow block, and the CGPG engine may be configured to convert the at least one unspecified foundation block to at least one specified foundation block based on the user specification, and generate the encoded clinical guidance protocol including the at least one specified foundation block and the at least one workflow block connected by the sequencing links. The at least one workflow block may be associated with one or more of specific clinical procedure or specific patient condition. The at least one workflow block may be a built-in workflow or an imported workflow. The built-in workflow may include one of a bi-level ventilation workflow, a continuous positive airway pressure (CPAP) ventilation workflow, a spirometry workflow, a non-rebreather (NRC) mask supplemental oxygen workflow, or a nasal cannula supplemental oxygen workflow. The imported workflow block may be an encoded clinical guidance protocol previously generated at the CGPG GUI. Re-use of previously generated encoded clinical guidance protocols may provide the technical advantage of importing and proliferating medically acceptable and customized protocols and sub-protocols, as constructed using the tools described herein, without requiring each customized protocol construction to start from a completely unspecified group of protocol blocks. Further, the tool described herein enables the combination of previously generated and specified protocol blocks with newly generated and specified blocks to further enhance the customization capabilities of the system. The encoded clinical guidance protocol previously generated at the CGPG GUI may include one of a spinal cord immobilization workflow, an acute respiratory failure (ARF) workflow, a modified early warning score (MEWS) workflow, a multi-threaded score evaluation workflow, a CPR advisory workflow, or a trend monitoring protocol. The at least one specified foundation block may be an initiation block for the at least one workflow block. The CGPG engine may be configured to incorporate the user specification into the at least one unspecified protocol block to convert the at least one unspecified protocol block to the at least one specified protocol block, and the at least one specified protocol block may be configured to cause the clinical guidance engine to implement the encoded clinical guidance protocol according to the incorporated user specification. The at least one unspecified protocol block may include (a) an operator and (b) an undefined operation and the at least one specified protocol block may include (a) the operator and (b) a defined operation based on the user specification. The operator may be configured to cause the clinical guidance engine to execute the defined operation as a background task without generating a clinical guidance UI control. The operator may include one of an evaluation or a command for the clinical guidance engine. The evaluation may include a conditional evaluation or a range evaluation. The command may include a refresh command or a timer command. The operator may include a value assignment operator configured to define a value of a parameter or define a variable. The operator may include an internal logic operator for the clinical guidance engine. The operator may include a guidance operator and the unspecified protocol block may include at least one instruction operator, at least one reference operator, or a series of operators including at least one instruction operator and at least on reference operator. The operator may be configured to cause the clinical guidance engine to generate a clinical guidance UI control to execute the defined operation. The operator may be configured cause the clinical guidance engine generate to generate one of: (a) a delay clinical guidance UI control, (b) a user input clinical guidance UI control, (c) a drug administration clinical guidance UI control, (d) a caregiver instruction clinical guidance UI control, (e) a multiple-choice clinical guidance UI control, (f) an alert clinical guidance UI control, (g) a guidance request control, or (h) a checklist clinical guidance UI control. The drug administration clinical guidance UI control may include one or more of a drug name display, a dosage display, a number of doses display, dose count, a dose interval timer, a delivery confirmation control, or a delivery cancellation control. The clinical guidance UI may be configured to implement at least the portion of the encoded clinical guidance protocol with medical device information for at least one medical device communicatively coupled to a device providing the clinical guidance UI.

The CGPG engine may be configured to receive a user entry of an encoded clinical guidance protocol attribute including one or more of a protocol name, a protocol input, a protocol output, or a protocol variable. The protocol input may include an input to the encoded clinical guidance protocol from the clinical guidance engine. The protocol output may include an output from the encoded clinical guidance protocol to the clinical guidance engine. The clinical guidance engine may execute the encoded clinical guidance protocol as a nested workflow block within an envelope protocol and the protocol output may be a value used in a subsequent protocol block of the envelope protocol. The protocol variable may depend on a context of the encoded clinical guidance protocol. The protocol variable may include a conditional operator for the clinical guidance engine.

Each of the plurality of protocol blocks may include (a) an entry linkage port and (b) one or more output linkage ports, and the user indications of the sequencing links may include user selections of (a) at least one output linkage port of a first protocol block of the plurality of protocol blocks, and (b) one entry linkage port of at least one second protocol block of the plurality of protocol blocks. The CGPG GUI may be configured to display each sequencing link as a line connecting the first protocol block and the at least one second protocol block. Each of the one or more output linkage ports may be associated with a sequencing attribute that indicates a sequencing relationship between the first protocol block and the at least one second protocol block. At least one protocol block of the plurality of protocol blocks may be a user input protocol block configured to generate a user input clinical guidance UI control, and the one or more output linkage ports comprise an entry confirmation port and an entry cancelation port. The one or more output linkage ports may include an advance port. A protocol block having the advance port may include one of (a) a wait time protocol block configured to cause the clinical guidance engine to generate a wait time clinical guidance UI control, (b) a drug administration protocol block configured to cause the clinical guidance engine to generate a drug administration clinical guidance UI control, (c) an alert protocol block configured to cause the clinical guidance engine to generate an alert clinical guidance UI control, or (d) a checklist protocol block configured to cause the clinical guidance engine to generate a checklist clinical guidance UI control, or (e) an instruction block comprising a guidance output port and configured to cause the clinical guidance engine to generate a request guidance UI control. At least one protocol block of the plurality of protocol blocks may be a UI instruction protocol block configured to generate a caregiver instruction clinical guidance UI control, and the one or more output linkage ports may include an advance port and a guidance port. At least one protocol block of the plurality of protocol blocks may be a multiple-choice protocol block configured to generate a multiple-choice clinical guidance UI control, and the one or more output linkage ports comprise respective ports for each choice option. At least one protocol block of the plurality of protocol blocks may be a protocol block configured to configured to cause the clinical guidance engine to execute conditional evaluation without generating a clinical guidance UI control, and the one or more output linkage ports may include a yes port, a no port, and an unknown outcome port. At least one protocol block of the plurality of protocol blocks may be a range evaluation protocol block configured to cause the clinical guidance engine to execute a range evaluation without generating a clinical guidance UI control, and the one or more output linkage ports comprise respective port for each range option and an unknown outcome port. At least one protocol block of the plurality of protocol blocks may be a timer protocol block configured to cause the clinical guidance engine to execute a timer command without generating a clinical guidance UI control, and the one or more output linkage ports comprise an advance port and an expiration port. At least one protocol block of the plurality of protocol blocks may be a refresh protocol block configured to cause the clinical guidance engine to obtain an updated parameter value from a medical device without generating a clinical guidance UI control, and the one or more output linkage ports comprise an advance port. At least one protocol block of the plurality of protocol blocks may be an internal logic protocol block configured to cause the clinical guidance engine to execute an internal protocol operation without generating a clinical guidance UI control, and the one or more output linkage ports comprise an advance port. The CGPG engine may be configured to validate the user specification and the sequencing links prior to an export of the encoded clinical guidance protocol. The CGPG engine may be configured to validate the user specification and sequencing links based on validation information retrieved from one or more libraries by the CGPG engine, the one or more libraries comprising one or more of an instructional library, a medication library, a physiological indications library, a medical device library, a protocol library, or a user account information library. The validation may include at least one of a first test that the user specification may be actionable and physiologically valid, or a second test that the sequencing links provide for a sequence of protocol blocks that may be actionable and physiologically valid, and the validation may generate a corrective action if either of the first test or the second test fail. In other examples, the corrective action is not generated. Instead, the validation generates an output indicative of the user specification being one or both of unactionable or physiologically invalid if the first test fails, and the validation generates an output indicative of the sequencing links being one or both of unactionable or physiologically invalid if the second test fails. The corrective action may include one or more of an automated correction with a prompt for user confirmation or a prompt for a user correction. The first test may confirm that a value for a parameter provided in the user specification conforms to a format of the parameter. The validation may confirm multiple choice loops indicated by one or more of the user specification or the sequencing links. The validation may confirm drug administrations indicated by one or more of the user specification or the sequencing links. The validation may confirm medical device instructions indicated by one or more of the user specification or the sequencing links. The validation may include a comparison of one or more of the connected protocol blocks to predetermined information in a medication library, which may include at least one of a medication indication library and a medication dosage library. The CGPG engine may be configured to retrieve medication specification guidelines from the medication library. The validation may include comparing one or more of the connected protocol blocks to predetermined information in a physiological indications library, which may include one or more of a physiological parameter library, a patient symptom and condition library, or a threshold and ranges library. The validation may provide for identification of invalid combinations of protocol blocks and/or sequencing links therebetween. The user specification and the sequencing links may define a closed loop control sequence for a medical device, and the CGPG engine may be configured to export the encoded clinical guidance protocol to the clinical guidance engine with an instruction to execute the closed loop control sequence, receive a parameter summary from the closed loop control sequence, and provide a corrective action based on the parameter summary.

The CGPG engine may be configured to store the encoded clinical guidance protocol at a platform resource manager communicatively coupled to the CGPG engine. The encoded clinical guidance protocol may be a data interchange format file. The encoded clinical guidance protocol may be a web application code file or an executable image file.

The CGPG GUI may be configured to provide a protocol block selection menu, a graphic protocol display area, and a user specification window. The protocol block selection menu may display the plurality of protocol blocks available for selection. The CGPG engine may be configured to receive the user selection of the plurality of protocol blocks via a drag and drop operations from the protocol block selection menu to the graphic protocol display area. The plurality of protocol blocks available for selection at the protocol block selection menu may include foundation blocks and workflow blocks. The foundation blocks may include protocol blocks that perform a background task without generating a clinical guidance UI control. The foundation blocks may include one or more of a condition evaluation protocol block, a range evaluation protocol block, a value assignment protocol block, a timer protocol block, a delay protocol block, a computation protocol block, a trend protocol block, or a refresh protocol block. The foundation blocks may include protocol blocks that generate a clinical guidance UI control. The foundation blocks may include one or more of an alert protocol block, a checklist protocol block, a multiple-choice protocol block, a medication administration protocol block, a UI instruction protocol block, a reference protocol block, or a UI user input protocol block. The workflow blocks may include built-in protocol blocks. The built-in protocol blocks may include one or more of a bi-level ventilation workflow block, a continuous positive airway pressure (CPAP) ventilation workflow block, a spirometry workflow block, a supplemental oxygen with non-rebreather mask workflow block, and a supplemental oxygen with nasal cannula workflow block. The workflow blocks may include encoded clinical guidance protocols previously generated at the CGPG GUI. The encoded clinical guidance protocol may include the workflow blocks nested within an envelope protocol based on selections from the protocol block selection menu. The CGPG GUI may be configured to display graphic representations of the user selected plurality of protocol blocks in the graphic protocol display area. The CGPG GUI may be configured to display graphic representations of the sequencing links in the graphic protocol display area. The CGPG engine may be configured to provide the user specification window in a format based on the user selected plurality of protocol blocks and receive the user specification as entries to the user specification window. The format may provide for one or more of at least one fillable field or at least one drop down menu.

At least one protocol block of the plurality of protocol blocks may be configured to cause the clinical guidance engine to utilize data from at least one medical device configured to communicatively couple to the clinical guidance engine. The data may include one or more of a patient status parameter or a device status parameter. The user specification may include a type of data and a type of medical device. The user specification may include a medical device make and model.

The CGPG engine may be configured to store the encoded clinical guidance protocol at a platform resource manager. The clinical guidance engine may be disposed at a device including a display, the device including a mobile computing device or a medical device, and may be configured to communicatively couple to a cloud server providing the clinical guidance protocol platform, receive the exported encoded clinical guidance protocol, and provide the clinical guidance UI at least in part at the mobile computing device or the medical device. The clinical guidance engine may be disposed at the cloud server and may be configured to communicatively couple to a device including a display, the device including a mobile computing device or a medical device, host a clinical guidance application at the device, and provide the clinical guidance UI at least in part at the display via the hosted clinical guidance application.

The system may include at least one resource manager communicatively coupled to the CGPG engine and the clinical guidance engine. The at least one resource manager may include at least one of a platform resource manager, a local resource manager, or an agency resource manager. The at least one resource manager may include a platform resource manager and at least one of a local resource manager or an agency resource manager and wherein the local resource manager and the agency resource manager may be configured to synchronize with the platform resource manager. The encoded clinical guidance protocol may be associated with user account information, and the clinical guidance engine may be configured to store the encoded clinical guidance protocol in the at least one resource manager according to the user account information. The at least one resource manager may include one or more of an instructional library, a medication library, a physiological indications library, a medical device library, a protocol library, or a user account information library. The instructional library may include one or more of an instructional image library, an instructional animation library, or an instructional text library. The plurality of protocol blocks may include at least one protocol block configured to cause the clinical guidance engine to retrieve instructional information from the instructional library and provide the instructional information at the clinical guidance UI. The at least one protocol block may be configured to cause the clinical guidance engine to retrieve medical device information from the medical device library, the medical device information being indicative of one or more medical devices communicatively coupled to the clinical guidance engine, and retrieve the instructional information based on an association with the one or more medical devices communicatively coupled to the clinical guidance engine. The medication library may include at least one of a medication indication library and a medication dosage library. The CGPG engine may be configured to retrieve medication specification guidelines from the medication library and provide the medication specification guidelines at the CGPG GUI. The CGPG engine may be configured to validate the user specification based on information in the medication library. The plurality of protocol blocks may include at least one protocol block configured to cause the clinical guidance engine to retrieve medication information from the medication library and provide the medication information at the clinical guidance UI. The physiological indications library may include one or more of a physiological parameter library, a patient symptom and condition library, or a threshold and ranges library. The CGPG engine may be configured to retrieve specification guidelines for physiological parameters from the physiological indications library and provide the specification guidelines for physiological parameters at the CGPG GUI. The CGPG engine may be configured to validate the user specification based on information in the physiological indications library. The plurality of protocol blocks may include at least one protocol block configured to cause the clinical guidance engine to retrieve physiological parameter information from the physiological indications library and provide the physiological parameter information at the clinical guidance UI. The medical device library may include one or more of a medical device drivers library or a verified medical devices library. The plurality of protocol blocks may include at least one protocol block configured to cause the clinical guidance engine to provide an instruction to at least one medical device based on information in the medical device library. The instruction may include an instruction to the at least one medical device to perform one or more of a therapeutic function or a monitoring function. The therapeutic function may include providing a therapeutic treatment or changing a therapeutic treatment parameter. The monitoring function may include initiating monitoring of a physiological parameter of the patient. The monitoring function may include monitoring the physiological parameter of the patient for a pre-determined time interval. The instruction to the at least one medical device may include a closed loop control instruction. The plurality of protocol blocks may include at least one protocol block configured to cause the clinical guidance engine to receive and evaluate data from at least one medical device without generating a clinical guidance UI control and based on information in the medical device library. The plurality of protocol blocks may include at least one protocol block configured to cause the clinical guidance engine to provide caregiver guidance for at least one medical device at the clinical guidance UI. The verified medical devices library may indicate medical devices authorized to interact with the clinical guidance engine. The protocol library may include the encoded clinical guidance protocol. The system may include a protocol customization engine configured to enable customization of at least one encoded clinical guidance protocol and save the customized at least one encoded clinical guidance protocol in a customized protocol library of the protocol library. The user account information library may include user account information associated with the encoded clinical guidance protocol. The user account information library may include user account information associated with one or more of the instructional library, the medication library, the physiological indications library, the medical device library, and the protocol library. The encoded clinical guidance protocol may include a monitor block configured to cause the clinical guidance engine to implement the encoded clinical guidance protocol as a monitoring protocol. The encoded clinical guidance protocol may include at least one variable and the monitor block may be configured to cause the clinical guidance engine to detect a change in a value of the at least one variable and implement the encoded clinical guidance protocol in response to the detected change in value. The encoded clinical guidance protocol may include a first workflow sequenced to a second workflow, wherein the first workflow may generate a parameter output and the second workflow may evaluate the parameter output at one or more constituent protocol blocks. The parameter output may include a trend evaluation for a physiological parameter. The trend evaluation may include one of improving, deteriorating, or stable.

The encoded clinical guidance protocol may include a spinal cord assessment workflow. The CGPG engine may be configured to receive the user selection at the CGPG GUI of the plurality of protocol blocks including unspecified multiple-choice protocol blocks and unspecified UI instruction protocol blocks, receive the user specification at the CGPG GUI for the unspecified multiple-choice protocol blocks and the unspecified UI instruction protocol blocks, convert the unspecified multiple-choice protocol blocks and the unspecified instruction blocks to specified multiple-choice protocol blocks and specified UI instruction protocol blocks based on the user specification, receive the user indications at the CGPG GUI of the sequencing links between the specified multiple-choice protocol blocks and the specified UI instruction protocol blocks, connect the specified multiple-choice protocol blocks and the specified UI instruction protocol blocks according to the user-indicated sequencing links between the specified multiple-choice protocol blocks and the specified UI instruction protocol blocks, generate the spinal cord assessment workflow including the specified multiple-choice protocol blocks and the specified UI instruction protocol blocks connected by the user-indicated sequencing links, and export the generated spinal cord assessment workflow to the clinical guidance engine configured to implement the spinal cord assessment workflow at the clinical guidance UI. The specified multiple-choice protocol blocks may cause the clinical guidance engine to provide a plurality of spinal evaluation criteria at a clinical guidance UI, receive caregiver entries at the clinical guidance UI indicative of a presence or absence of each of the plurality of spinal evaluation criteria, and provide a caregiver instruction at the clinical guidance UI based on the caregiver entries. The sequencing links between the specified multiple-choice protocol blocks and the specified instruction protocol blocks may cause the clinical guidance engine to provide a first instruction if all of the plurality of spinal evaluation criteria are present and to provide a second instruction of any of the spinal evaluation criteria are absent. The first instruction may be for spinal motion restriction and the second instruction may be to bypass spinal motion restriction. At least one of the first instruction and the second instruction provide a caregiver selectable option for further guidance at the clinical guidance UI.

The encoded clinical guidance protocol may include a modified early warning score (MEWS) assessment workflow. The CGPG engine may be configured to receive the user selection at the CGPG GUI of the plurality of protocol blocks including (a) a plurality of first foundation protocol blocks configured to cause the clinical guidance engine to execute a background task without generating a clinical guidance UI control, and (b) a plurality of second foundation protocol blocks configured to cause the clinical guidance engine to generate a UI control at the clinical guidance UI, receive the user specification at the CGPG GUI for unspecified first and second foundation blocks, convert the unspecified first and second foundation blocks to specified first and second foundation blocks based on the user specification, receive the user indications at the CGPG GUI of the sequencing links between the specified first and second foundation blocks, connect the specified first and second foundation blocks according to the user-indicated sequencing links, generate the MEWS assessment workflow including the specified foundation protocol blocks connected by the user-indicated sequencing links, and export the MEWS assessment workflow to the clinical guidance engine configured to implement the MEWS assessment workflow at the clinical guidance UI. The first foundation protocol blocks may include medical device interaction protocol blocks. The medical device interaction protocol blocks may be configured to cause the clinical guidance engine to retrieve MEWS assessment physiological parameters from a communicatively coupled medical device. Each medical device interaction protocol block may include an indeterminate value output linkage port and may be configured to default to the indeterminate value output linkage port if a respective MEWS assessment physiological parameter is unavailable, and the sequencing links connect the indeterminate value output linkage port to an input linkage port of a protocol block configured to cause the clinical guidance UI to generate user prompt at the clinical guidance UI for a caregiver entry of the respective MEWS assessment physiological parameter. The MEWS assessment physiological parameters comprise non-invasive blood pressure (NIBP), heart rate, respiration rate, and temperature. The medical device interaction protocol blocks may include range evaluation protocol blocks. The first foundation protocol blocks may include internal logic protocol blocks. The internal logic protocol blocks may include value assignment protocol blocks and at least one computation protocol block. The internal logic protocol blocks may be configured to assign variable values for variables corresponding to MEWS assessment components. The second foundation protocol blocks may include UI instruction protocol blocks and at least one multiple-choice protocol block.

The encoded clinical guidance protocol may include an acute respiratory failure (ARF) workflow. The CGPG engine may be configured to receive the user selection at the CGPG GUI of the plurality of protocol blocks including foundation protocol blocks and a bi-level ventilation built-in workflow block, receive the user specification at the CGPG GUI for unspecified foundation protocol blocks, convert the unspecified foundation protocol blocks to specified foundation protocol blocks based on the user specification, receive the user indications at the CGPG GUI of the sequencing links between the specified foundation protocol blocks and between the foundation protocol blocks and the bi-level ventilation built-in workflow block, connect the specified foundation protocol blocks and the bi-level ventilation built-in workflow block according to the user-indicated sequencing links, generate the ARF workflow including the specified foundation protocol blocks and the bi-level ventilation built-in workflow block connected by the user-indicated sequencing links, and export the ARF workflow to the clinical guidance engine configured to implement the ARF workflow at the clinical guidance UI. The foundation protocol blocks may include (a) a plurality of first foundation protocol blocks configured to cause the clinical guidance engine to execute a background task without generating a clinical guidance UI control, and (b) a plurality of second foundation protocol blocks configured to cause the clinical guidance engine to generate a UI control at the clinical guidance UI. The first foundation protocol blocks may include medical device interaction protocol blocks. The medical device interaction protocol blocks may be configured to cause the clinical guidance engine to retrieve respiratory parameters from a communicatively coupled medical device. The first foundation protocol blocks may include internal logic protocol blocks. The internal logic protocol blocks may be configured to assign variable values for ventilation parameters. The internal logic protocol blocks may be configured to define delay times in the ARF workflow to account for delayed patient responses to ventilation interventions. The internal logic protocol blocks may include value assignment protocol blocks and delay protocol blocks. The second foundation protocol blocks may be configured to cause the clinical guidance engine to generate a caregiver instruction. The second foundation protocol blocks may be configured to cause the clinical guidance engine to generate a caregiver instruction for a medication. The second foundation protocol blocks may include one or more of UI instruction protocol blocks or medication administration protocol blocks.

The encoded clinical guidance protocol may include a cardiopulmonary resuscitation (CPR) advisory workflow. The CPR advisory workflow may include (a) one or more specified protocol blocks configured to provide chest compression guidance, (b) one or more specified protocol blocks configured to provide ventilation guidance, and (c) one or more specified protocol blocks configured to evaluate an ECG rhythm and provide shock delivery guidance based on the ECG rhythm evaluation. The CPR advisory workflow may include one or more specified protocol blocks configured to evaluate return of spontaneous circulation (ROSC) and provide guidance based on the ROSC evaluation. The CPR advisory workflow may include one or more specified protocol blocks configured to evaluate an amplitude spectrum analysis (AMSA) value and provide defibrillation shock instructions based at least in part on the AMSA value evaluation. The CGPG engine may enable a user selection of AMSA score evaluation tools. The user selected AMSA score evaluation tools may include one or more of a conditional evaluation of an AMSA score value, a conditional evaluation of a fractional change in the AMSA score value, or a conditional evaluation of an AMSA value trend. The CGPG engine may enable user entry of conditional evaluation metrics for the one or more of the AMSA score evaluation tools. The CPR advisory workflow may be configured to enable the clinical guidance engine to receive at least one of a ROSC indication, an ECG rhythm analysis, or a chest compression count from a communicatively coupled medical device and/or medical sensor. The CPR advisory workflow may include at least one drug administration protocol block. The CPR advisory workflow may include at least one protocol block configured to count chest compressions and at least one protocol block configured to count chest compression cycles. The CPR advisory workflow may include conditional evaluations of the counted chest compressions and counted chest compression cycles and provides for one or more of shock delivery, compression delivery, ventilation delivery, or drug delivery based on the conditional evaluations.

Each sequencing link may connect an output port of a first protocol block with an input port of a second protocol block, the CGPG GUI may be configured to graphically represent each sequencing link, and the CGPG engine may be configured to assign a first unique identifier to the output port, assign a second unique identifier to the input port, and encode the graphically represented sequencing link as a sequencing instruction in the encoded clinical guidance protocol using the first and the second unique identifiers. The encoded clinical guidance workflow may be a multi-threaded workflow configured to cause the clinical guidance engine to evaluate a plurality of medical evaluation scores in parallel.

An example of a method for generating encoded clinical guidance protocols includes receiving, by a clinical guidance protocol generation (CGPG) engine a user selection at a CGPG graphical user interface (GUI) of a plurality of protocol blocks comprising at least one unspecified protocol block, receiving, by the CGPG engine, a user specification at the CGPG GUI for the at least one unspecified protocol block, converting, by the CGPG engine, the at least one unspecified protocol block to at least one specified protocol block based on the user specification, receiving, by the CGPG engine, a user indication at the CGPG GUI of sequencing links between the plurality of protocol blocks, connecting, by the CGPG engine, the plurality of protocol blocks according to the user-indicated sequencing links between the plurality of protocol blocks, generating, by the CGPG engine, an encoded clinical guidance protocol comprising the plurality of protocol blocks connected by the sequencing links between the plurality of protocol blocks, validating, by the CGPG engine, encoded clinical guidance protocol, and, based on the validation, exporting, by the CGPG engine the encoded clinical guidance protocol to a database configured for access by a clinical guidance engine configured to implement at least a portion of the encoded clinical guidance protocol at a clinical guidance user interface (UI).

Implementations of such a method may include one or more of the following features. The method may include exporting the encoded clinical guidance protocol if the validation is satisfied, providing a notification at the CGPG GUI, and enabling a user of the CGPG GUI to return to at least one of selecting the plurality of protocol blocks, specifying at least one unspecified protocol block, or indicating a sequencing link if the validation is unsatisfied. The validating may include at least one of verifying that the user specification is actionable and physiologically valid or verifying that the sequencing links provide for a sequence of protocol blocks that is actionable and physiologically valid. Each sequencing link may connect an output port of a first protocol block with an input port of a second protocol block and the method may include graphically representing each sequencing link at the CGPG GUI, assigning a first unique identifier to the output port, assigning a second unique identifier to the input port, and encoding the graphically represented sequencing link as a sequencing instruction to generate the encoded clinical guidance protocol using the first and the second unique identifiers. Receiving the user indication of the sequencing links may include receiving a user selection at the CGPG GUI of the output port from one or more output ports associated with the first protocol block and receiving a user selection of the input port at the CGPG GUI. The method may include displaying each sequencing link as a line connecting the selected output port of the first protocol block and the selected input port of the second protocol block. The at least one unspecified protocol block may include at least one unspecified foundation block, the plurality of protocol blocks may include at least one workflow block, and the method may include converting the at least one unspecified foundation block to at least one specified foundation block based on the user specification, and generating the encoded clinical guidance protocol comprising the at least one specified foundation block and the at least one workflow block connected by the sequencing links. The at least one workflow block may be associated with one or more of specific clinical procedure or specific patient condition. The method may include providing the at least one workflow block at the CGPG GUI as a built-in workflow, wherein the built-in workflow is a workflow provided with the database. The method may include providing the at least one workflow block at the CGPG GUI as an imported workflow, wherein the imported workflow is a workflow previously generated at the CGPG GUI and saved in the database. The method may include incorporating the user specification into the at least one unspecified protocol block to convert the at least one unspecified protocol block to the at least one specified protocol block, wherein the at least one specified protocol block is configured to cause the clinical guidance engine to implement the encoded clinical guidance protocol according to the incorporated user specification. The method may include displaying the at least one unspecified protocol block with (a) an operator and (b) an undefined operation and displaying the at least one specified protocol block with (a) the operator and (b) a defined operation based on the user specification. The operator may be one of a conditional evaluation operator, a range evaluation operator, a refresh operator, a timer operator, an internal logic operator, or an operator configured to generate a clinical guidance UI control. The method may include receiving a user entry of an encoded clinical guidance protocol attribute comprising one or more of a protocol name, a protocol input, a protocol output, or a protocol variable. The protocol input may include an input to the encoded clinical guidance protocol from the clinical guidance engine. The protocol output may include an output from the encoded clinical guidance protocol to the clinical guidance engine. The method may include storing the encoded clinical guidance protocol in the database as a data interchange format file. The method may include storing the encoded clinical guidance protocol in the database as one of a web application code file or an executable image file. The method may include providing a protocol block selection menu, a graphic protocol display area, and a user specification window at the CGPG GUI. The method may include displaying graphic representations of the user selected plurality of protocol blocks in the graphic protocol display area. The method may include displaying graphic representations of the sequencing links in the graphic protocol display area. The method may include providing the user specification window in a format based on the user selected plurality of protocol blocks and receiving the user specification as entries to the user specification window. The format may provide for one or more of at least one fillable field or at least one drop down menu. The database may include at least one resource manager and the method may include storing the encoded clinical guidance protocol at the at least one resource manager. The at least one resource manager may include at least one of a platform resource manager, a local resource manager, or an agency resource manager. The method may include associating the encoded clinical guidance protocol with user account information and storing encoded clinical guidance protocol in the at least one resource manager according to the user account information. The clinical guidance engine may be disposed at a device comprising a display, the device comprising a mobile computing device or a medical device and the method may include communicatively coupling, by the clinical guidance engine, to the database, receiving, by the clinical guidance engine, the exported encoded clinical guidance protocol, and providing the clinical guidance UI at least in part at the display. The clinical guidance engine may be disposed at a cloud server and the method may include communicatively coupling to a device comprising a display, the device comprising a mobile computing device or a medical device, hosting a clinical guidance application at the device, and providing the clinical guidance UI at least in part at the display via the hosted clinical guidance application. The encoded clinical guidance protocol may include one of a spinal cord assessment workflow, a modified early warning score (MEWS) assessment workflow, an acute respiratory failure (ARF) workflow, a CPR advisory workflow, or a multi-threaded medical evaluation score workflow.

An example of a system for generating encoded clinical guidance protocols includes a clinical guidance protocol generation (CGPG) graphical user interface (GUI), and at least one non-transitory, processor-readable storage medium having stored thereon processor-readable instructions for providing a clinical guidance protocol generation platform, the processor-readable instruction being configured to cause at least one processor to receive a user selection at the CGPG GUI of a plurality of protocol blocks comprising at least one unspecified protocol block, receive a user specification at the CGPG GUI for the at least one unspecified protocol block, convert the at least one unspecified protocol block to at least one specified protocol block based on the user specification, receive user indications at the CGPG GUI of sequencing links between the plurality of protocol blocks, connect the plurality of protocol blocks according to the user-indicated sequencing links, generate an encoded clinical guidance protocol comprising the plurality of protocol blocks connected by the user-indicated sequencing links, and export the encoded clinical guidance protocol to a computing device configured to implement at least a portion of the encoded clinical guidance protocol at a clinical guidance user interface (UI).

Implementations of such a system may include one or more of the following features. The at least one unspecified protocol block may include at least one unspecified foundation block, the plurality of protocol blocks may include at least one workflow block, and the processor-readable instructions may be configured to cause the at least one processor to convert the at least one unspecified foundation block to at least one specified foundation block based on the user specification, and generate the encoded clinical guidance protocol comprising the at least one specified foundation block and the at least one workflow block connected by the sequencing links. The at least one workflow block may be associated with one or more of specific clinical procedure or specific patient condition. The at least one workflow block may include a built-in workflow or an imported workflow. The built-in workflow may include one of a bi-level ventilation workflow, a continuous positive airway pressure (CPAP) ventilation workflow, a spirometry workflow, a non-rebreather (NRC) mask supplemental oxygen workflow, or a nasal cannula supplemental oxygen workflow. The imported workflow may include an encoded clinical guidance protocol previously generated at the CGPG GUI. The encoded clinical guidance protocol previously generated at the CGPG GUI may include one of a spinal cord immobilization workflow, an acute respiratory failure (ARF) workflow, a modified early warning score (MEWS) workflow, a multi-threaded score evaluation workflow, a CPR advisory workflow, or a trend monitoring protocol. The at least one specified foundation block may be an initiation block for the at least one workflow block. The processor-readable instructions may be configured to cause the at least one processor to incorporate the user specification into the at least one unspecified protocol block to convert the at least one unspecified protocol block to the at least one specified protocol block, and the at least one specified protocol block may be configured to implement the encoded clinical guidance protocol according to the incorporated user specification. The at least one unspecified protocol block may include (a) an operator and (b) an undefined operation, and the at least one specified protocol block may include (a) the operator and (b) a defined operation based on the user specification. The operator may be configured to cause processor execution of the defined operation as a background task without generating a clinical guidance UI control. The operator may include one of a processor-executable clinical guidance evaluation or a processor-executable clinical guidance command.

The evaluation may include a conditional evaluation or a range evaluation. The command may include a refresh command or a timer command. The operator may include a value assignment operator configured to define a value of a parameter or define a variable or an internal logic operator. The operator may include a guidance operator and the unspecified protocol block may include at least one instruction operator, at least one reference operator, or a series of operators comprising the at least one instruction operator and the at least one reference operator. The operator may be configured to generate a clinical guidance UI control to execute the defined operation. The operator may be configured to generate one of (a) a delay clinical guidance UI control, (b) a user input clinical guidance UI control, (c) a drug administration clinical guidance UI control, (d) a caregiver instruction clinical guidance UI control, (e) a multiple-choice clinical guidance UI control, (f) an alert clinical guidance UI control, (g) a guidance request control, or (h) a checklist clinical guidance UI control. The drug administration clinical guidance UI control may include one or more of a drug name display, a dosage display, a number of doses display, dose count, a dose interval timer, a delivery confirmation control, or a delivery cancellation control. The clinical guidance UI may be configured to implement at least the portion of the encoded clinical guidance protocol with medical device information for at least one medical device communicatively coupled to a device providing the clinical guidance UI. The processor-readable instructions may be configured to cause the at least one processor may be configured to receive a user entry of an encoded clinical guidance protocol attribute comprising one or more of a protocol name, a protocol input, a protocol output, or a protocol variable. The encoded clinical guidance protocol may be configured for processor-execution as a nested workflow block within an envelope protocol and the protocol output may be a value used in a subsequent protocol block of the envelope protocol. The protocol variable depends on a context of the encoded clinical guidance protocol. The protocol variable may include a conditional operator. Each of the plurality of protocol blocks may include (a) an entry linkage port and (b) one or more output linkage ports, and the user indications of the sequencing links may include user selections of (a) at least one output linkage port of a first protocol block of the plurality of protocol blocks, and (b) one entry linkage port of at least one second protocol block of the plurality of protocol blocks. The CGPG GUI may be configured to display each sequencing link as a line connecting the first protocol block and the at least one second protocol block. Each of the one or more output linkage ports may be associated with a sequencing attribute that indicates a sequencing relationship between the first protocol block and the at least one second protocol block. At least one protocol block of the plurality of protocol blocks may be a user input protocol block configured to generate a user input clinical guidance UI control, and the one or more output linkage ports may include an entry confirmation port and an entry cancelation port. The one or more output linkage ports may include an advance port. A protocol block having the advance port may include one of (a) a wait time protocol block configured to generate a wait time clinical guidance UI control, (b) a drug administration protocol block configured to generate a drug administration clinical guidance UI control, (c) an alert protocol block configured to generate an alert clinical guidance UI control, (d) a checklist protocol block configured to generate a checklist clinical guidance UI control, or (e) an instruction block comprising a guidance output port and configured to generate a request guidance UI control. At least one protocol block of the plurality of protocol blocks may be a UI instruction protocol block configured to generate a caregiver instruction clinical guidance UI control, and the one or more output linkage ports may include an advance port and a guidance port. At least one protocol block of the plurality of protocol blocks may be a multiple-choice protocol block configured to generate a multiple-choice clinical guidance UI control, and the one or more output linkage ports may include respective ports for each choice option. At least one protocol block of the plurality of protocol blocks may be a protocol block configured to configured to evaluate a condition without generating a clinical guidance UI control, and the one or more output linkage ports may include a yes port, a no port, and an unknown outcome port. At least one protocol block of the plurality of protocol blocks may be a range evaluation protocol block configured to evaluate a range without generating a clinical guidance UI control, and the one or more output linkage ports may include respective port for each range option and an unknown outcome port. At least one protocol block of the plurality of protocol blocks may be a timer protocol block configured to implement a timer without generating a clinical guidance UI control, and the one or more output linkage ports may include an advance port and an expiration port. At least one protocol block of the plurality of protocol blocks may be a refresh protocol block configured to obtain an updated parameter value from a medical device without generating a clinical guidance UI control, and the one or more output linkage ports may include an advance port. At least one protocol block of the plurality of protocol blocks may be an internal logic protocol block configured to cause processor-execution of an internal protocol operation without generating a clinical guidance UI control, and the one or more output linkage ports may include an advance port. The processor-readable instructions may be configured to cause the at least one processor to validate the user specification and the sequencing links prior to an export of the encoded clinical guidance protocol. The processor-readable instructions may be configured to cause the at least one processor to validate the user specification and the sequencing links based on validation information retrieved from one or more libraries, the one or more libraries comprising one or more of an instructional library, a medication library, a physiological indications library, a medical device library, a protocol library, or a user account information library. The validation may include at least one of a first test that the user specification may be actionable and physiologically valid, or a second test that the sequencing links provide for a sequence of protocol blocks that may be actionable and physiologically valid, and wherein the validation generates a corrective action if either of the first test or the second test fail. The corrective action may include one or more of an automated correction with a prompt for user confirmation or a prompt for a user correction. The first test confirms that a value for a parameter provided in the user specification conforms to a format of the parameter. The validation may include at least one of a first test that the user specification may be actionable and physiologically valid, or a second test that the sequencing links provide for a sequence of protocol blocks that may be actionable and physiologically valid, and, respectively, the validation generates an output indicative of the user specification being one or both of unactionable or physiologically invalid if the first test fails, and the validation generates an output indicative of the sequencing links being one or both of unactionable or physiologically invalid if the second test fails. The validation confirms at least one of multiple-choice loops indicated by one or more of the user specification or the sequencing links, drug administrations indicated by one or more of the user specification or the sequencing links, or medical device instructions indicated by one or more of the user specification or the sequencing links. The user specification and the sequencing links define a closed loop control sequence for a medical device, and the processor-readable instructions may be configured to cause the at least one processor to export the encoded clinical guidance protocol with an instruction to execute the closed loop control sequence, receive a parameter summary from the closed loop control sequence, and provide a corrective action based on the parameter summary. The processor-readable instructions may be configured to cause the at least one processor to store the encoded clinical guidance protocol at a platform resource manager. The encoded clinical guidance protocol may be a data interchange format file. The encoded clinical guidance protocol may be one of a web application code file or an executable image file. The CGPG GUI may be configured to provide a protocol block selection menu, a graphic protocol display area, and a user specification window. The protocol block selection menu may display the plurality of protocol blocks available for selection. The processor-readable instructions may be configured to cause the at least one processor to receive the user selection of the plurality of protocol blocks via a drag and drop operations from the protocol block selection menu to the graphic protocol display area. The plurality of protocol blocks available for selection at the protocol block selection menu may include foundation blocks and workflow blocks. The foundation blocks may include protocol blocks that perform a background task without generating a clinical guidance UI control. The foundation blocks may include one or more of a condition evaluation protocol block, a range evaluation protocol block, a value assignment protocol block, a timer protocol block, a delay protocol block, a match protocol block, a computation protocol block, a trend protocol block, or a refresh protocol block. The foundation blocks may include protocol blocks that generate a clinical guidance UI control. The foundation blocks may include one or more of an alert protocol block, a checklist protocol block, a multiple-choice protocol block, a medication administration protocol block, a UI instruction protocol block, a reference protocol block, or a UI user input protocol block. The workflow blocks may include built-in protocol blocks. The built-in protocol blocks may include one or more of a bi-level ventilation workflow block, a continuous positive airway pressure (CPAP) ventilation workflow block, a spirometry workflow block, a supplemental oxygen with non-rebreather mask workflow block, and a supplemental oxygen with nasal cannula workflow block. The workflow blocks may include encoded clinical guidance protocols previously generated at the CGPG GUI. The encoded clinical guidance protocol may include the workflow blocks nested within an envelope protocol based on selections from the protocol block selection menu. The CGPG GUI may be configured to display graphic representations of the user selected plurality of protocol blocks in the graphic protocol display area. The CGPG GUI may be configured to display graphic representations of the sequencing links in the graphic protocol display area. The processor-readable instructions may be configured to cause the at least one processor to provide the user specification window in a format based on the user selected plurality of protocol blocks and receive the user specification as entries to the user specification window. The format provides for one or more of at least one fillable field or at least one drop down menu. At least one protocol block of the plurality of protocol blocks may be configured to utilize data from at least one medical device. The data may include one or more of a patient status parameter or a device status parameter. The user specification may include a type of data and a type of medical device. The user specification may include a medical device make and model. The processor-readable instructions may be configured to cause the at least one processor to store the encoded clinical guidance protocol at a platform resource manager. The at least one processor may be communicatively coupled to at least one device comprising a mobile computing device or a medical device, the at least one device being configured to communicatively couple to a cloud server providing the clinical guidance protocol platform, receive the exported encoded clinical guidance protocol, and provide the clinical guidance UI at least in part at the display. The clinical guidance UI may be provided via a clinical guidance application hosted by a cloud server. The system may include least one resource manager communicatively coupled to the at least one processor. The at least one resource manager may include at least one of a platform resource manager, a local resource manager, or an agency resource manager. The at least one resource manager may include a platform resource manager and at least one of a local resource manager or an agency resource manager and wherein the local resource manager and the agency resource manager may be configured to synchronize with the platform resource manager. The encoded clinical guidance protocol may be associated with user account information, and the at least one resource manager may be configured to store the encoded clinical guidance protocol according to the user account information. The at least one resource manager may include one or more of an instructional library, a medication library, a physiological indications library, a medical device library, a protocol library, or a user account information library. The instructional library may include one or more of an instructional image library, an instructional animation library, or an instructional text library. The plurality of protocol blocks may include at least one protocol block configured to provide instructional information retrieved from the instructional library at the clinical guidance UI. The at least one protocol block may be configured to retrieve the instructional information based on an association with one or more medical devices communicatively coupled to a device hosting the clinical guidance UI. The medication library may include at least one of a medication indication library and a medication dosage library. The processor-readable instructions may be configured to cause the at least one processor to retrieve medication specification guidelines from the medication library and provide the medication specification guidelines at the CGPG GUI. The processor-readable instructions may be configured to cause the at least one processor to validate the user specification based on information in the medication library. The plurality of protocol blocks may include at least one protocol block configured to provide the medication information retrieved from the medication library at the clinical guidance UI. The physiological indications library may include one or more of a physiological parameter library, a patient symptom and condition library, or a threshold and ranges library. The processor-readable instructions may be configured to cause the at least one processor to retrieve specification guidelines for physiological parameters from the physiological indications library and provide the specification guidelines for physiological parameters at the CGPG GUI. The processor-readable instructions may be configured to cause the at least one processor to validate the user specification based on information in the physiological indications library. The plurality of protocol blocks may include at least one protocol block configured to provide the physiological parameter information retrieved from the physiological indications library at the clinical guidance UI. The medical device library may include one or more of a medical device drivers library or a verified medical devices library. The plurality of protocol blocks may include at least one protocol block configured to provide an instruction to at least one medical device based on information in the medical device library. The instruction may include an instruction to the at least one medical device to perform one or more of a therapeutic function or a monitoring function. The therapeutic function may include providing a therapeutic treatment or changing a therapeutic treatment parameter. The monitoring function may include initiating monitoring of a physiological parameter of a patient. The monitoring function may include monitoring the physiological parameter of the patient for a pre-determined time interval. The instruction to the at least one medical device may include a closed loop control instruction. The plurality of protocol blocks may include at least one protocol block configured to receive and evaluate data from at least one medical device without generating a clinical guidance UI control and based on information in the medical device library. The plurality of protocol blocks may include at least one protocol block configured to provide caregiver guidance for at least one medical device at the clinical guidance UI. The verified medical devices library indicates medical devices authorized to interact with a device hosting the clinical guidance UI. The protocol library may include the encoded clinical guidance protocol. The system may include at least one processor configured to enable customization of at least one encoded clinical guidance protocol and save the customized at least one encoded clinical guidance protocol in a customized protocol library of the protocol library. The user account information library may include user account information associated with the encoded clinical guidance protocol. The user account information library may include user account information associated with one or more of the instructional library, the medication library, the physiological indications library, the medical device library, and the protocol library. The encoded clinical guidance protocol may include a monitor block configured to implement the encoded clinical guidance protocol as a monitoring protocol. The encoded clinical guidance protocol may include at least one variable and the monitor block may be configured to detect a change in a value of the at least one variable and implement the encoded clinical guidance protocol in response to the detected change in value. The encoded clinical guidance protocol may include a first workflow sequenced to a second workflow, wherein the first workflow generates a parameter output, and the second workflow evaluates the parameter output at one or more constituent protocol blocks. The parameter output may include a trend evaluation for a physiological parameter. The trend evaluation may include one of improving, deteriorating, or stable. The encoded clinical guidance protocol may include a spinal cord assessment workflow. The processor-readable instructions may be configured to cause the at least one processor to receive the user selection at the CGPG GUI of the plurality of protocol blocks comprising unspecified multiple-choice protocol blocks and unspecified UI instruction protocol blocks, receive the user specification at the CGPG GUI for the unspecified multiple-choice protocol blocks and the unspecified UI instruction protocol blocks, convert the unspecified multiple-choice protocol blocks and the unspecified instruction blocks to specified multiple-choice protocol blocks and specified UI instruction protocol blocks based on the user specification, receive the user indications at the CGPG GUI of the sequencing links between the specified multiple-choice protocol blocks and the specified UI instruction protocol blocks, connect the specified multiple-choice protocol blocks and the specified UI instruction protocol blocks according to the user-indicated sequencing links between the specified multiple-choice protocol blocks and the specified UI instruction protocol blocks, generate the spinal cord assessment workflow comprising the specified multiple-choice protocol blocks and the specified UI instruction protocol blocks connected by the user-indicated sequencing links, and export the generated spinal cord assessment workflow to a device configured to provide the clinical guidance UI. The specified multiple-choice protocol blocks may be configured to provide a plurality of spinal evaluation criteria at a clinical guidance UI, receive caregiver entries at the clinical guidance UI indicative of a presence or absence of each of the plurality of spinal evaluation criteria, and provide a caregiver instruction at the clinical guidance UI based on the caregiver entries. The sequencing links between the specified multiple-choice protocol blocks and the specified instruction protocol blocks may be configured to provide a first instruction if all of the plurality of spinal evaluation criteria may be present and to provide a second instruction of any of the spinal evaluation criteria may be absent. The first instruction may be for spinal motion restriction and the second instruction may be to bypass spinal motion restriction. At least one of the first instruction and the second instruction may provide a caregiver selectable option for further guidance at the clinical guidance UI. The encoded clinical guidance protocol may include a modified early warning score (MEWS) assessment workflow. The processor-readable instructions may be configured to cause the at least one processor to receive the user selection at the CGPG GUI of the plurality of protocol blocks comprising (a) a plurality of first foundation protocol blocks configured to execute a background task without generating a clinical guidance UI control, and (b) a plurality of second foundation protocol blocks configured to generate a UI control at the clinical guidance UI, receive the user specification at the CGPG GUI for unspecified first and second foundation blocks, convert the unspecified first and second foundation blocks to specified first and second foundation blocks based on the user specification, receive the user indications at the CGPG GUI of the sequencing links between the specified first and second foundation blocks, connect the specified first and second foundation blocks according to the user-indicated sequencing links, generate the MEWS assessment workflow comprising the specified foundation protocol blocks connected by the user-indicated sequencing links, and export the MEWS assessment workflow to a device associated with the clinical guidance UI. The plurality of first foundation protocol blocks may include medical device interaction protocol blocks. The medical device interaction protocol blocks may be configured to retrieve MEWS assessment physiological parameters from a communicatively coupled medical device. Each medical device interaction protocol block may include an indeterminate value output linkage port and may be configured to default to the indeterminate value output linkage port if a respective MEWS assessment physiological parameter may be unavailable, and the sequencing links connect the indeterminate value output linkage port to an input linkage port of a protocol block configured to cause the clinical guidance UI to generate user prompt at the clinical guidance UI for a caregiver entry of the respective MEWS assessment physiological parameter. The MEWS assessment physiological parameters may include non-invasive blood pressure (NIBP), heart rate, respiration rate, and temperature. The medical device interaction protocol blocks may include range evaluation protocol blocks. The plurality of first foundation protocol blocks may include internal logic protocol blocks. The internal logic protocol blocks may include value assignment protocol blocks and at least one computation protocol block. The internal logic protocol blocks may be configured to assign variable values for variables corresponding to MEWS assessment components. The plurality of second foundation protocol blocks may include UI instruction protocol blocks and at least one multiple-choice protocol block. The encoded clinical guidance protocol may include an acute respiratory failure (ARF) workflow. The processor-readable instructions may be configured to cause the at least one processor to receive the user selection at the CGPG GUI of the plurality of protocol blocks comprising foundation protocol blocks and a bi-level ventilation built-in workflow block, receive the user specification at the CGPG GUI for unspecified foundation protocol blocks, convert the unspecified foundation protocol blocks to specified foundation protocol blocks based on the user specification, receive the user indications at the CGPG GUI of the sequencing links between the specified foundation protocol blocks and between the foundation protocol blocks and the bi-level ventilation built-in workflow block, connect the specified foundation protocol blocks and the bi-level ventilation built-in workflow block according to the user-indicated sequencing links, generate the ARF workflow comprising the specified foundation protocol blocks and the bi-level ventilation built-in workflow block connected by the user-indicated sequencing links, and export the ARF workflow to a device associated with the clinical guidance UI. The foundation protocol blocks may include (a) a plurality of first foundation protocol blocks configured to execute a background task without generating a clinical guidance UI control, and (b) a plurality of second foundation protocol blocks configured to generate a UI control at the clinical guidance UI. The plurality of first foundation protocol blocks may include medical device interaction protocol blocks. The medical device interaction protocol blocks may be configured to retrieve respiratory parameters from a communicatively coupled medical device. The plurality of first foundation protocol blocks may include internal logic protocol blocks. The internal logic protocol blocks may be configured to assign variable values for ventilation parameters. The internal logic protocol blocks may be configured to define delay times in the ARF workflow to account for delayed patient responses to ventilation interventions. The internal logic protocol blocks may include value assignment protocol blocks and delay protocol blocks. The plurality of second foundation protocol blocks may be configured to generate a caregiver instruction. The plurality of second foundation protocol blocks may be configured to generate the caregiver instruction for a medication. The plurality of second foundation protocol blocks may include one or more of UI instruction protocol blocks or medication administration protocol blocks. The encoded clinical guidance protocol may include a cardiopulmonary resuscitation (CPR) advisory workflow. The CPR advisory workflow may include (a) one or more specified protocol blocks configured to provide chest compression guidance, (b) one or more specified protocol blocks configured to provide ventilation guidance, and (c) one or more specified protocol blocks configured to evaluate an ECG rhythm and provide shock delivery guidance based on the ECG rhythm evaluation. The CPR advisory workflow may include one or more specified protocol blocks configured to evaluate return of spontaneous circulation (ROSC) and provide guidance based on the ROSC evaluation. The CPR advisory workflow may include one or more specified protocol blocks configured to evaluate an amplitude spectrum analysis (AMSA) value and provide defibrillation shock instructions based at least in part on the AMSA value evaluation. The processor-readable instructions may be configured to provide a user selection of AMSA score evaluation tools. The user selected AMSA score evaluation tools may include one or more of a conditional evaluation of an AMSA score value, a conditional evaluation of a fractional change in the AMSA score value, or a conditional evaluation of an AMSA value trend. The processor-readable instructions may be configured to cause the at least one processor to capture user entry of conditional evaluation metrics for the one or more of the AMSA score evaluation tools. The CPR advisory workflow may be configured to incorporate at least one of a ROSC indication, an ECG rhythm analysis, or a chest compression count from a communicatively coupled medical device and/or medical sensor. The CPR advisory workflow may include at least one drug administration protocol block. The CPR advisory workflow may include at least one protocol block configured to count chest compressions and at least one protocol block configured to count chest compression cycles. The CPR advisory workflow may include conditional evaluations of the counted chest compressions and counted chest compression cycles and provides for one or more of shock delivery, compression delivery, ventilation delivery, or drug delivery based on the conditional evaluations. Each sequencing link connects an output port of a first protocol block with an input port of a second protocol block, the CGPG GUI may be configured to graphically represent each sequencing link, and the processor-readable instructions may be configured to cause the at least one processor to assign a first unique identifier to the output port, assign a second unique identifier to the input port, and encode the graphically represented sequencing link as a sequencing instruction in the encoded clinical guidance protocol using the first and the second unique identifiers.

Other capabilities may be provided and not every implementation according to the disclosure must provide any, let alone all, of the capabilities discussed. Further, it may be possible for an effect noted above to be achieved by means other than that noted, and a noted item/technique may not necessarily yield the noted effect.

### BRIEF DESCRIPTION OF THE DRAWINGS

The accompanying drawings, which are incorporated in and constitute a part of the specification, illustrate one or more embodiments and, together with the description, explain these embodiments. The accompanying drawings have not necessarily been drawn to scale. Any values and/or dimensions illustrated in the accompanying graphs and figures are for illustration purposes only and may or may not represent actual or preferred values or dimensions. Where applicable, some or all features may not be illustrated to assist in the description of underlying features.

FIG. 1 shows examples of patient care environments.

FIGS. 2A and 2B show an example of a system for generating an encoded clinical guidance protocol.

FIG. 3 shows an example of a system for generating and customizing an encoded clinical guidance protocol.

FIG. 4A shows examples of medical devices and patient interface devices.

FIG. 4B shows an example of a communications configuration for the CGPG system.

FIG. 4C shows examples of medical device communications.

FIG. 5A shows an example of a basic layout for a CGPG graphical user interface (GUI).

FIG. 5B shows an example of a CGPG graphical user interface (GUI).

FIG. 5C shows an example of a protocol generated with foundation blocks and workflow blocks.

FIG. 5D shows examples of assigning clinical guidance protocol attributes.

FIG. 5E shows examples of assigning clinical guidance protocol attributes for a nested workflow.

FIG. 5F shows examples of parameters used to generate encoded clinical guidance protocols.

FIG. 6 shows an example of a foundation protocol block conversion from unspecified to specified.

FIG. 7 shows examples of input and output ports and sequencing links as used during protocol generation.

FIG. 8A-1 shows examples of a range check operator and a match evaluation operator.

FIG. 8A-2 shows examples of a condition evaluation operator and a value assignment operator.

FIGS. 8B-1 and 8B-2 show examples of a timer operator, a computation operator, and a delay operator.

FIG. 8C shows examples of an alert operator and a check list operator.

FIG. 8D shows examples of a multiple-choice operator and a reference operator.

FIG. 8E-1 shows an example of a trend operator.

FIG. 8E-2 shows examples of a UI instruction operator and a UI user input operator.

FIG. 8E-3 shows an example of a medication administration operator.

FIG. 8E-4 shows an example of UI features corresponding to a medication administration operator in an encoded clinical guidance protocol.

FIG. 8E-5 shows an example of a protocol generation sequence with a medication administration operator.

FIG. 8F-1 shows an example of guidance operator.

FIG. 8F-2 shows an example of tiered clinical guidance UI features corresponding to guidance and reference operators in an encoded clinical guidance protocol.

FIGS. 8G-8H show examples of foundation protocol blocks and corresponding user specification windows.

FIGS. 81-1-81-3 show examples of clinical guidance UI features corresponding to encoded clinical guidance protocol specifications.

FIG. 9 shows an example of parallel processing of multiple initiation blocks by the clinical guidance engine.

FIG. 10 shows an example of a method of generating an encoded clinical guidance protocol.

FIG. 11 shows an example of a validation error message.

FIG. 12 shows an example of a resource manager for a clinical guidance protocol generation and clinical guidance system.

FIG. 13 shows an example of a parameter file from the parameter library.

FIG. 14 shows an example of an encoded spinal cord assessment protocol generated at the CGPG GUI.

FIGS. 15A-15E show an example of an encoded modified early warning score (MEWS) assessment protocol generated at the CGPG GUI.

FIG. 16 shows an example of an encoded clinical guidance protocol that evaluates several different medical evaluation scores.

FIGS. 17A-17B show an example of an encoded acute respiratory failure (ARF) protocol generated at the CGPG GUI.

FIGS. 18A-18B show an example of an encoded CPR advisory protocol generated at the CGPG GUI.

FIGS. 19A-19C show an example of an encoded EtCO2 trend monitoring protocol generated at the CGPG GUI.

### DETAILED DESCRIPTION

The description set forth below in connection with the appended drawings is intended to be a description of various, illustrative embodiments or implementations of the disclosed subject matter. Specific features and functionalities are described in connection with each illustrative embodiment or implementation; however, it will be apparent to those skilled in the art that the disclosed embodiments and implementations may be practiced without each of those specific features and functionalities.

Reference throughout the specification to “one embodiment,” “an embodiment,” or “an implementation” means that a particular feature, structure, or characteristic described in connection with an embodiment or implementation is included in at least one embodiment or implementation of the subject matter disclosed. Thus, the appearance of the phrases “in one embodiment” or “in an embodiment” or “in an implementation” in various places throughout the specification is not necessarily referring to the same embodiment or implementation. Further, the particular features, structures or characteristics may be combined in any suitable manner in one or more embodiments or implementations. Further, it is intended that embodiments and implementations of the disclosed subject matter cover modifications and variations thereof.

It must be noted that, as used in the specification and the appended claims, the singular forms “a,” “an,” and “the” include plural referents unless the context expressly dictates otherwise. That is, unless expressly specified otherwise, as used herein the words “a,” “an,” “the,” and the like carry the meaning of “one or more.” Additionally, it is to be understood that terms such as “left,” “right,” “top,” “bottom,” “front,” “rear,” “side,” “height,” “length,” “width,” “upper,” “lower,” “interior,” “exterior,” “inner,” “outer,” and the like that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation. The terms “workflow” and “protocol” are used interchangeably.

Furthermore, the terms “approximately,” “about,” “proximate,” “minor variation,” and similar terms generally refer to ranges that include the identified value within a margin of 20%, 10% or preferably 5% in certain embodiments, and any values there between.

All of the functionalities described in connection with one embodiment or implementation are intended to be applicable to the additional embodiments and implementations described below except where expressly stated or where the feature or function is incompatible with the additional embodiments and implementations. For example, where a given feature or function is expressly described in connection with one embodiment or implementation but not expressly mentioned in connection with an alternative embodiment or implementation, it should be understood that that feature or function may be deployed, utilized or implemented in connection with the alternative embodiment or implementation unless the feature or function is incompatible with the alternative embodiment or implementation.

Aspects of the present disclosure are directed to systems and methods for generating clinical guidance protocols for use in a clinical guidance system.

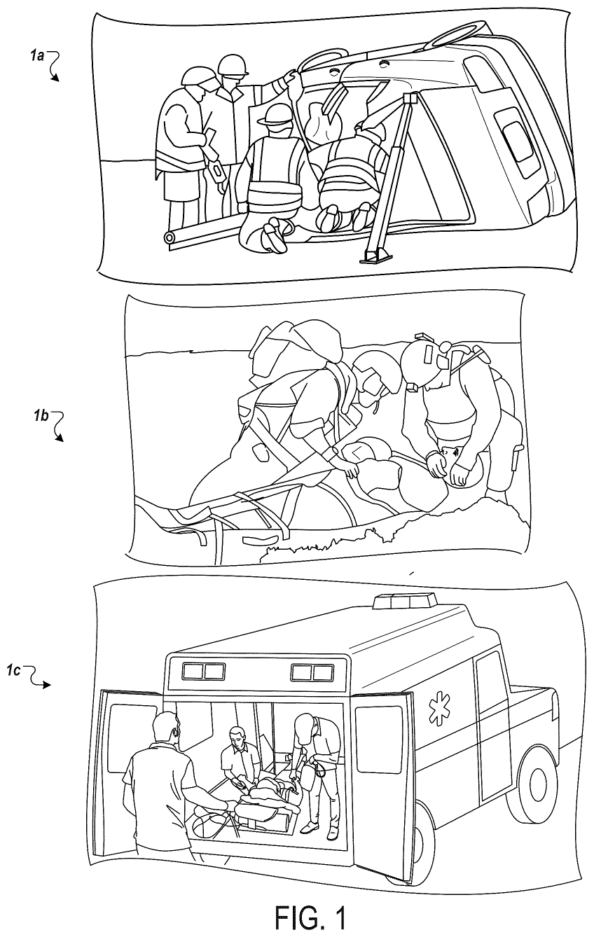

Referring to FIG. 1, examples of patient care environments are shown. Such environments may include a rescue scene, a military scene, or a transport vehicle scene, to name a few examples. The rescue scene 1a may be a scene of a car crash or another on-site trauma rescue scene such as a sporting event field, a blast site, a scene of a fall, etc. The military medical scene 13b may be on or near a battlefield, in a bay of a field hospital, in a military triage area, etc. The transport vehicle scene 1c may be inside an ambulance, as shown, or in a helicopter or other transport vehicle. The patient care environment is not limited to the physical scenes shown and may also include a hospital emergency room, an urgent care clinic, a rural field hospital, an emergency medical tent, a military medical facility or field hospital, etc. In many situations, connectivity to a long-range communication network, such as a cellular or computer network, may be unavailable and/or undesirable. For example, areas such as urban canyons, parking garages, remote rural locations, interior spaces, military field locations, etc. may lack or have unstable network connectivity. In military environments, connectivity may be undesirable. Such connectivity may provide a means for tracking and locating personnel by enemy forces.

Regardless of the specific physical location, the patient care environment may be a chaotic and crowded environment with many distractions in which acute critical care is necessary with a smaller choice of medical devices than might be available at a large hospital, for example. This may be particularly true in an emergency care situation where care providers, often first responders, responders for trauma cannot monitor a patient's condition for many hours, request lengthy lab or imaging procedures, or comb through a comprehensive medical history. Rather these caregivers are tasked with making accurate split second and potentially lifesaving, decisions in the emergency environment. There may be multiple caregivers and/or multiple patients crowded into a small area. A clinical guidance system that can adapt the guidance based on the context of the patient and the patient care environment may improve patient outcomes.

In order to provide sufficiently adaptable guidance, a clinical guidance system may provide a capability for a medical director or other clinician or medical care provider with limited and minimal knowledge of computer programming, and certainly without any expertise in this field, to readily generate a computer-implemented clinical guidance tool. As described herein, such a user may manipulate tools on a user-friendly and intuitive graphic user interface to design and customize a clinical guidance protocol. This user may interact directly with the graphical design tool to create a graphical representation of a clinical guidance protocol. A backend of this graphic design tool encodes this graphical representation such that a computer processor can provide clinical guidance according to the encoded clinical guidance protocol in real-time during patient care. Such an interface enables the medical personnel to personally design, specify, adjust, and create a computer-implementable clinical guidance protocol without working through a third-party programmer. Without such a tool, medical personnel would have to describe their needs to a programmer and iteratively prototype and test the results in a long, indirect, and relatively expensive process. The system described herein bypasses such a process. A medical expert can arrange and specify their protocol according to their needs and preferences without working through a third-party computer programming expert. Furthermore, the system described herein enables the clinical guidance protocol to be automatically tested and validated without trial runs during patient care and enables such a tool to operate with or without network connectivity.

The system described herein generates clinical guidance protocols specifically designed for use in the medical care situations described above. The encoded clinical guidance protocols best enhance patient outcomes when such protocols provide instructions and guidance in a manner that may minimize or alleviate caregiver distraction and confusion. The clinical guidance protocol generation system described herein enables a guidance framework that specifies what a clinical guidance system may do on its own without interaction from the user of the clinical guidance system during a patient encounter. The system described herein accomplishes this in part due to a capability to instruct or control medical devices. For example, the generated protocols may enable closed-loop control of various medical devices, information requests from the medical devices, information storage at the medical devices, determination of operational settings, inventory of available devices, and clinical guidance tailored to specific medical devices available during patient care.

In medical treatment situations, caregivers are often inundated with information. The system described herein generates clinical guidance protocols that enable the caregiver to off-load the intake, the analysis, and the determination of the most efficient and effective treatments and to receive guidance in providing that treatment. By minimizing caregiver interactions with a clinical guidance tool, the tool may provide beneficial guidance without distracting or confusing the caregiver. The encoded clinical guidance protocols generated herein do not merely enable a presentation of an outline steps that conform to an industry standard of care. Rather, these protocols provide for background tasks that may monitor multiple physiological parameters in parallel and then evaluate when these physiological parameters require attention, what conditions are implicated by these parameters, determine the caregiver guidance that is needed to treat conditions implicated, and determine what medical device instructions may enable that treatment. For example, these protocols may enable a guidance tool to run timers, refresh parameters, and provide conditional and range evaluations without any caregiver participation. Further, the generated protocols described herein are not limited to medical determinations but also include logistic guidance, for example, as prompts to set up or prepare various items of equipment along with providing guidance on how to operate that equipment and connect sensors to a patient. Accordingly, in one or more examples, the system for generating encoded clinical guidance protocols described herein may be configured to enable the generation of different encoded clinical guidance protocols based on the user selection, the user specification and the user indications, tailored to an intended use-case for the one or more medical devices that are to be controlled by the exported encoded clinical guidance protocol and which may provide the clinical guidance user interface. Further, in one or more examples, the system for generating encoded clinical guidance protocols described herein may be provided in combination with one or more or a plurality of medical devices configured to receive different exported encoded clinical guidance protocols generated by the system.

As a further advantage, because the clinical guidance system described herein enables the provision of a clinical guidance UI, this UI may be used during training and/or in the absence of communications with medical devices (e.g., due to connectivity issues) just based on the graphic depictions and access to information in resource manager libraries. As another example of the advantage of the system described herein, the system enables training within a real-time implementation of a clinical procedure. The timing of procedures and sequences of procedures provided by a real-time guidance system provides implementation guidance that is not available from a printed or memorized protocol implementation.

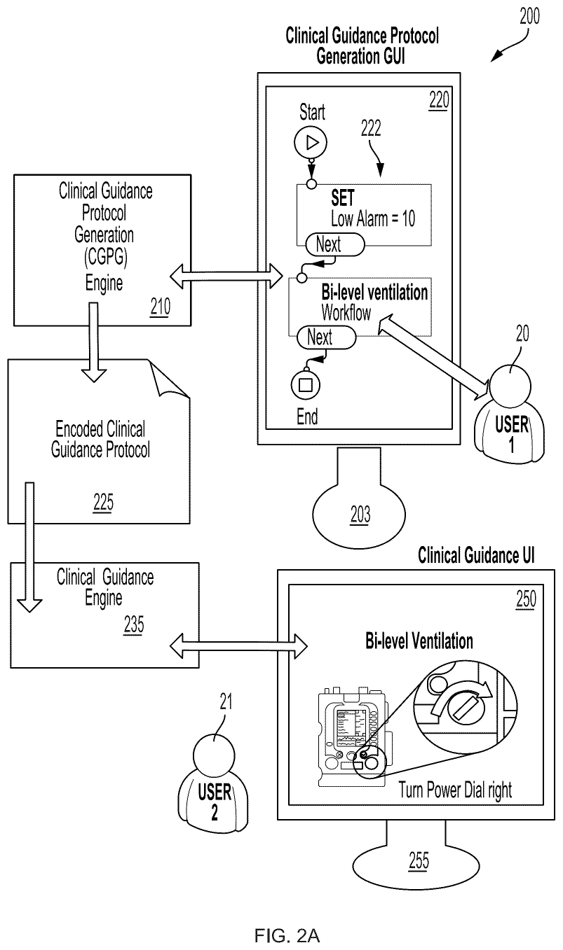

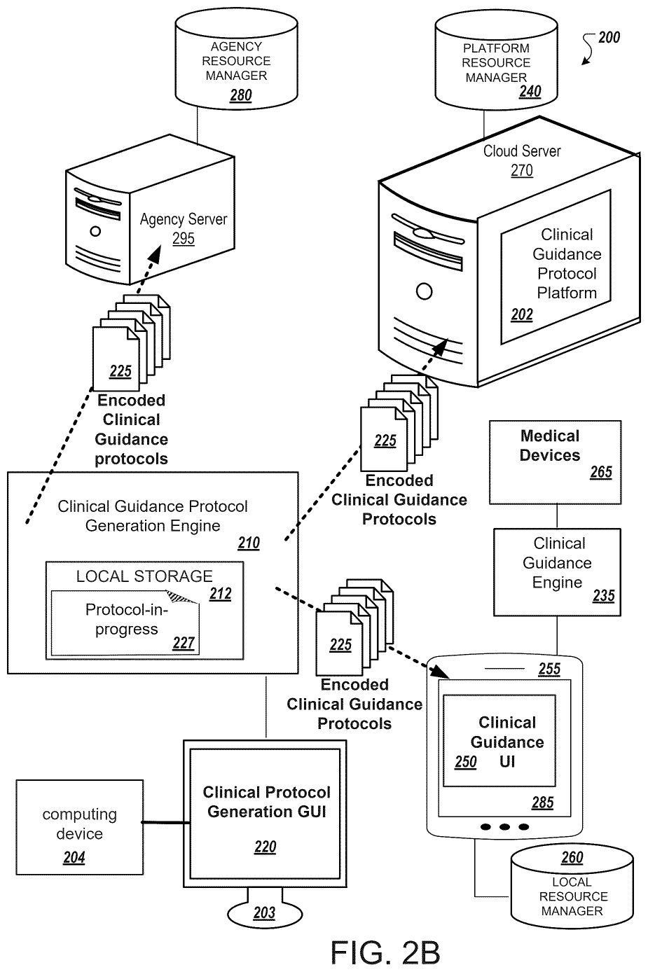

Referring to FIGS. 2A and 2B, an example of a system for generating an encoded clinical guidance protocol is shown. A quantity of each component in these figures is an example only and other quantities of each, or any, component could be used. The system 200 includes a clinical guidance protocol generation (CGPG) engine 210 and a clinical guidance engine 235. Although illustrated as separate engines herein, in some examples, the CGPG engine 210 and the clinical guidance engine 235 may be a single engine that combines the described functions of these two engines.

The engines described herein (e.g., the various engines shown in FIGS. 2A, 2B, 3, 4A, 4B, and 4Ca) may include hardware logic and/or software logic provided by one or more processors and/or memory to implement logic instructions to perform one or more tasks at one or more computing devices and/or at one or more medical devices. In some examples, the actions performed by engines may be implemented by hardware, software, firmware, middleware, microcode, hardware description languages, etc., or any combination thereof. When implemented in software, firmware, middleware, or microcode, the program code or code segments to perform the tasks may be stored in a non-transitory processor-readable medium such as a storage medium. Processors may perform the described tasks. In various implementations, the engines described herein may be or may include at least one processor and a non-transitory processor-readable storage medium having stored thereon processor-readable instructions configured to cause the at least one processor to perform actions according to the processor-readable instructions. The storage medium and/or the processor may be components of a computing device and/or a medical device.

The CGPG engine 210 includes hardware logic and/or software logic (e.g., as provided by a processor, a memory, and associated circuitry) that enables the CGPG engine 210 to provide and implement a CGPG graphical user interface (GUI) 220 at a display device 203. The CGPG GUI 220 enables a user (e.g., the first user 20) to create a graphical representation 222 of a clinical guidance protocol at the CGPG GUI 220. The CGPG engine 210 further includes hardware logic and/or software logic that enables the CGPG engine 210 to generate an encoded clinical guidance protocol 225 that represents and corresponds to the graphical representation 222. As described in further detail below, the system 200 enables validation of the encoded clinical guidance protocol 225. Additionally, the system 200 enables a user-friendly means for the user of the CGPG GUI 220 to edit, change, modify, and rearrange the protocols before they are exported to clinician devices. Overall, CGPG engine 210 may generate the encoded clinical guidance protocol 225 according to the method discussed below in regard to FIG. 10.

In an implementation, the encoded clinical guidance protocol 225 may be, for example, a data interchange format file (e.g., a JavaScript Object Notation (JSON) file, a data serialization file, a protocol buffer file, an encrypted binary file, etc.) that the clinical guidance engine 235 is configured to interpret and parse in order to provide the clinical guidance UI 250 and to perform background tasks in support of and to supplement the clinical guidance UI 250. This data interchange format file supports a system that may run without Internet connectivity. As another example, the encoded clinical guidance protocol 225 may be a web application code file (e.g., using a client-side scripting language such as, but not limited to, JavaScript) that enables the clinical guidance engine 235 to run as a web application to provide the clinical guidance UI 250. As a further example, the encoded clinical guidance protocol 225 may be an executable image file.

As described in more detail below, clinical guidance engine 235 includes hardware logic and/or software logic (e.g., as provided by a processor, a memory, and associated circuitry) that enables the clinical guidance engine 235 to receive and evaluate data from medical devices, provide instructions to medical devices, receive and evaluation caregiver input, and provide caregiver guidance at the clinical guidance UI 250, all according to the encoded clinical guidance protocol 225. The encoded clinical guidance protocol 225 is configured to enable the clinical guidance engine 235 to implement and/or execute the encoded protocol blocks within the encoded clinical guidance protocol 225. In other words, encoded clinical guidance protocol 225 and the clinical guidance engine 235 are mutually compatible so as to enable implementation and/or execution of the encoded clinical guidance protocol 225. As an engine configured to implement the encoded clinical guidance protocol 225, the clinical guidance engine 235 may function as an interpreter of the encoded clinical guidance protocol 225.

In some examples, a display device 255 may provide all or a portion of the clinical guidance UI 250. In some examples, other devices, such as audio, haptic, augmented reality, and/or virtual reality devices, and combinations thereof including combinations with a display device, may provide the clinical guidance UI 250. In an implementation, the user interface device 255 may be a mobile device such as, for example, a computer tablet or a smartphone. In an implementation, at least one of the medical devices 265 may include a display 285 that provides the clinical guidance UI 250.

The user of the CGPG GUI 220 (e.g., a first user 20) may be, for example, a medical device equipment manufacturer or a medical protocol governing body that may generate the encoded clinical guidance protocol 1225 for use by a caregiver. In contrast, the user (e.g., a second user 21) of the clinical guidance UI 250 may be a caregiver for whom the clinical guidance UI 250 provides real-time clinical guidance during a patient encounter. The protocol generation may occur in a different time and place than the protocol utilization and, therefore, a user of the CGPG GUI 220 may not be a user of the clinical guidance UI 250 and vice versa. The protocol must be generated prior to being implemented by the clinical guidance engine 235 and the protocol generation process does not occur in real-time during a patient encounter.

In some examples, although FIG. 2A shows the CGPG GUI 220 at a separate device from the clinical guidance UI 250, in some examples, these two interfaces could be provided at a single device. Thus, the first user 20 and the second user 21 may be a same user with the ability to both generate and utilize encoded clinical guidance protocols 225 at a same display device (e.g., the display device 203 or the display device 255).

Referring to FIG. 2B, the system 200 is illustrated in more detail. As shown in this figure, the CGPG engine 210 may be provided by a clinical guidance protocol platform 202 hosted, for example, at a cloud server 270. The CGPG engine 210 may host a CGPG application at a computing device 204 that includes or provides the display 203. Alternatively, the CGPG engine 210 may be locally implemented at the computing device 204. In such a local implementation, the CGPG engine 210 may include software and/or library resources downloaded from the clinical guidance protocol platform 202. Although one encoded clinical guidance protocol is illustrated in FIG. 2A, the CGPG engine 210 may generate multiple encoded clinical guidance protocols 225 at the CGPG GUI 220. As described in detail below, the CGPG engine 210 may validate the generated protocols. Validation ensures that the generated protocol does not violate any implementation limits of the clinical guidance engine 235 and/or provides workflow instructions to clinicians and/or medical devices that are within physiologically feasible limits. The validation may provide for identification of invalid combinations of protocol blocks and/or sequencing links therebetween and/or invalid user specifications provided in unspecified protocol blocks (e.g., the user specifications provided in the user specification window 506).

The clinical guidance protocol platform 202 may further include a platform resource manager 240. The platform resource manager 240 may include various libraries with information available to the CGPG engine 210 for generation of the encoded clinical guidance protocol 225 and to the clinical guidance engine 235 for implementation of the encoded clinical guidance protocol 225. In some implementations, the clinical guidance engine 235 may only access the platform resource manager 240 to update various elements of the local or agency resource managers (e.g., new medical device instructions, driver updates, etc.). The local resource manager 260 may also enable the clinical guidance engine 235 to run in an off-line mode at the device 255. This may enable the clinical guidance engine 235 to operate effectively in emergency care environments that often lack network connectivity (e.g., parking garages, remote rural locations, interior spaces, military field locations, etc.). In an implementation, where network connectivity is available, the protocol blocks that require information from the resource managers may acquire that data from either the local, agency, or platform resource managers. This may enable a smaller amount of information to be maintained at the local resource manager 260 and reduce the computational load on the computing device 255 providing the local resource manager 260. In an implementation, the CGPG engine 210 may be configured to export the encoded clinical guidance protocols 225 to one or more of the platform resource manager 240, the local resource manager 260, or the agency resource manager 280 for storage and use by the clinical guidance engine 235.

In an implementation, an agency server 295 may provide the agency resource manager 280. The agency server 295 may be associated with an EMS agency, an EMS agency system, a hospital, a hospital system, and/or another medical care facility or system of facilities. The agency server 295 may be an on-premises server or a cloud server but is separate from and distinct from the cloud server 270. The agency server 295 may communicate with the cloud server 270 in order to access the resource manager 240.

In some examples, the platform resource manager 240 may be communicatively coupled to one or more of a local resource manager 260 and/or an agency resource manager 280. Additionally, in an implementation, the local resource manager 260 may be communicatively coupled to the agency resource manager 280. The local resource manager 260 and/or the agency resource manager 280 may be communicatively coupled to one or more of the CGPG engine 210 and/or the clinical guidance engine 235.

The local resource manager 260 and the agency resource manager 280 may include various libraries with information available to the clinical guidance engine 235 for clinical guidance protocol implementation. In various examples, the local resource manager 260 and the agency resource manager 280 may include all or some of the libraries and protocols stored at and provided by the platform resource manager 240. In an implementation, the platform resource manager 240 may store all protocols and libraries available for any user of the clinical guidance protocol platform 202. This may include a plurality of EMS agencies, hospitals, and/or other health care providers. The agency resource manager 280 may be associated with an agency server (e.g., the agency server 295 shown in FIG. 2B) and may store a subset of the protocols and libraries available at the platform resource manager 240. The subset may include protocols and/or libraries that are specific to and/or customized for a particular EMS agency, hospital and/or other health care provider. The local resource manager 260 may be associated with a device used by an individual health care provider or a specific team of health care providers (e.g., the device 255) to access a clinical guidance UI 250. The local resource manager 260 may include a subset of the protocols and/or libraries available at the platform resource manager 240 and/or at the agency resource manager 280. In an implementation, the subset of protocols and/or libraries of the local resource manager 260 may be tailored to a specific type of care provided by the individual or team. For example, if an individual provider or team is basic life support (BLS) certified but not advanced life support (ALS) certified, then the protocols and/or libraries available at the local resource manager 260 associated with that individual's device 255 may be limited to BLS protocols and/or libraries.

While an encoded clinical guidance protocol 225 is under construction and/or prior to export by the CGPG engine 210, the CGPG engine 210 may store the incomplete and/or pre-export clinical guidance protocol (e.g., the protocol-in-progress 227) in a local storage 212. The clinical guidance engine 235 may not access, utilize, or otherwise implement protocols stored in the local storage 212. These protocols may not yet in a format that the clinical guidance engine 235 can interpret and implement. For example, the protocol-in-progress 227 may include information relevant to the CGPG GUI 220 that enables compatibility and interoperability between the protocol-in-progress and the CGPG GUI 220 during protocol construction. The clinical guidance engine 235 may not be configured to parse or execute these instructions. The CGPG engine 210 may strip these instructions upon export of a completed protocol 225. Additionally, these protocols may contain errors prior to validation and/or may be missing steps and/or specifications because they are still being edited and/or under construction. Once complete, the CGPG engine 210 may export the encoded clinical guidance protocol 225 to the platform resource manager 240 and/or may export the encoded clinical guidance protocol 225 to the clinical guidance engine 235.

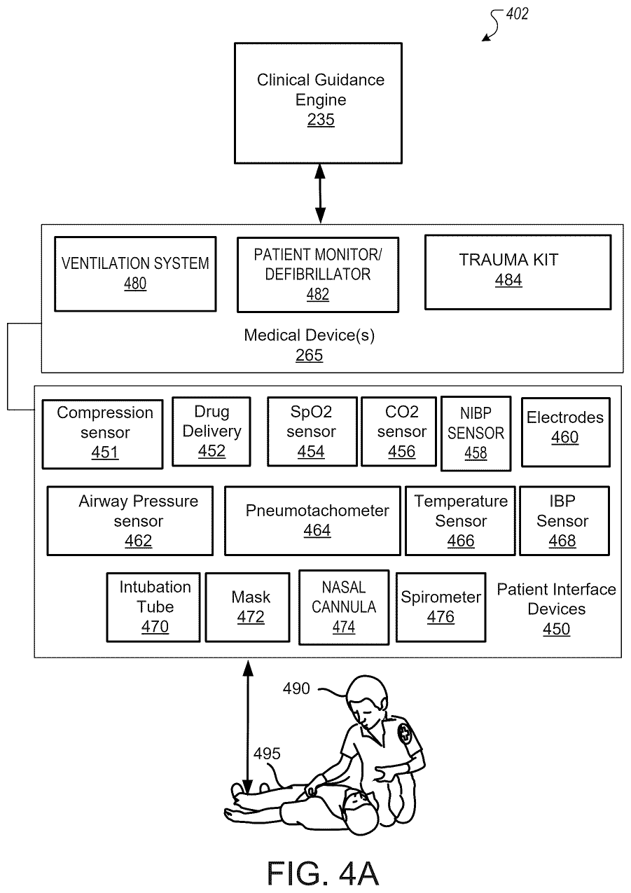

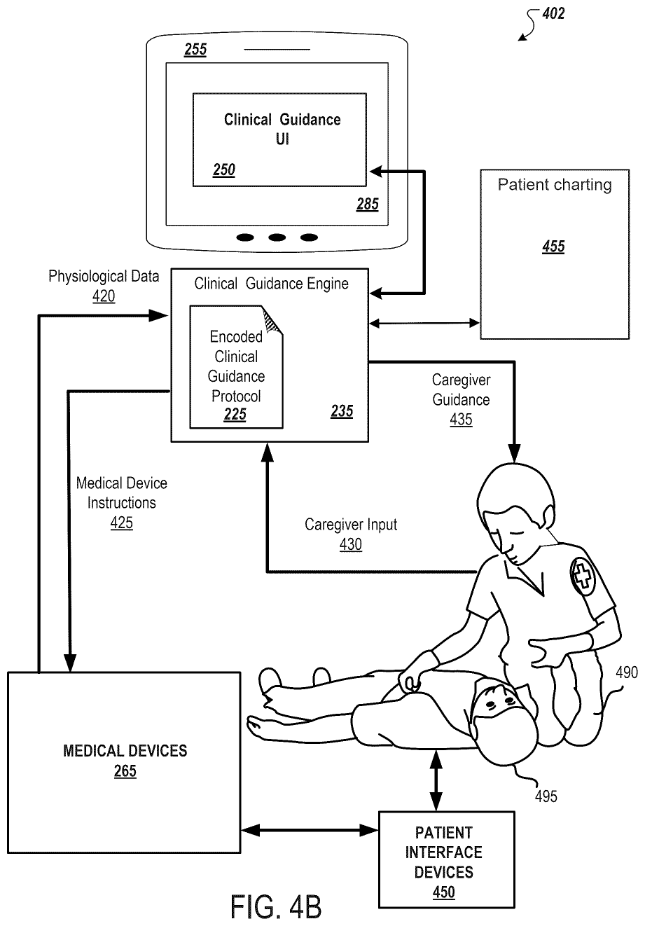

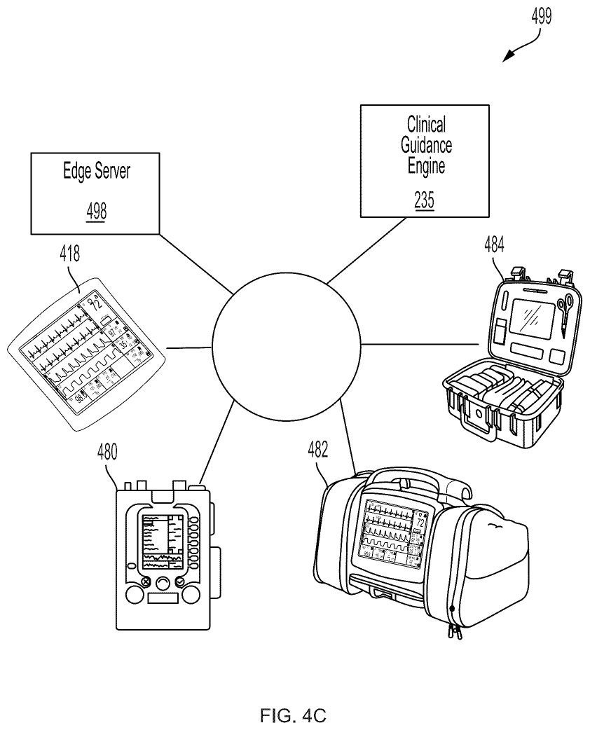

As described in more detail below with regard to FIG. 4B, implementation and/or execution of the protocol blocks of the encoded clinical guidance protocol 225 by the clinical guidance engine 235 may enable the provision of medical device instructions 425, the provision of caregiver guidance 435, the receipt of caregiver input 430, and/or the receipt and evaluation of physiological data 420. These tasks may be accomplished in conjunction with communications between the clinical guidance engine 235 and the resource managers 240, 260, and/or 280.

The clinical guidance engine 235 may be communicatively coupled to the one or more medical devices 265. The clinical guidance engine 235 may operate in a real-time mode during implementation of a generated protocol 225 at the clinical guidance UI 250 during care of a patient (e.g., care provided by the second user 21). The clinical guidance engine 235 may exchange information with and/or manage and control interactions with the medical devices 265. For example, the clinical guidance engine 235 may access device drivers in a resource manager (e.g., from the medical device library 1250 shown in FIG. 12) in order to communicate with and receive data from and/or exchange data with the medical devices 265.

The clinical guidance engine 235 may be disposed at one of the cloud server 270, the agency server 295, the user interface device 255, at least one of the medical devices 265, or may be a distributed resource across two or more of the cloud server 270, the agency server 295, the device 255, and the medical devices 265. The clinical guidance engine 235 may implement a portion of the encoded clinical guidance protocol 225 at a clinical guidance user interface (UI) 250. For example, the mobile computing device and/or the medical device may include a display 285 and/or another user interface device (e.g., an audio device, a haptic devices, and/or a virtual reality and/or an augmented reality display devices). The clinical guidance UI 250 may provide UI controls as directed by the clinical guidance engine 235 according to the encoded clinical guidance protocol 225. For example, UI controls may include caregiver entry prompts, caregiver instructions, alerts, etc. Additionally, the clinical guidance engine 235 may implement a portion of the encoded clinical guidance protocol 225 as background tasks without generating a UI control. For example, the background tasks may include receiving and evaluating data from one or more medical devices coupled to the patient. In this manner, the clinical guidance engine may minimize or alleviate caregiver distraction and confusion and conform the information provided to a caregiver to the specific context of a patient as indicated by the medical device data.

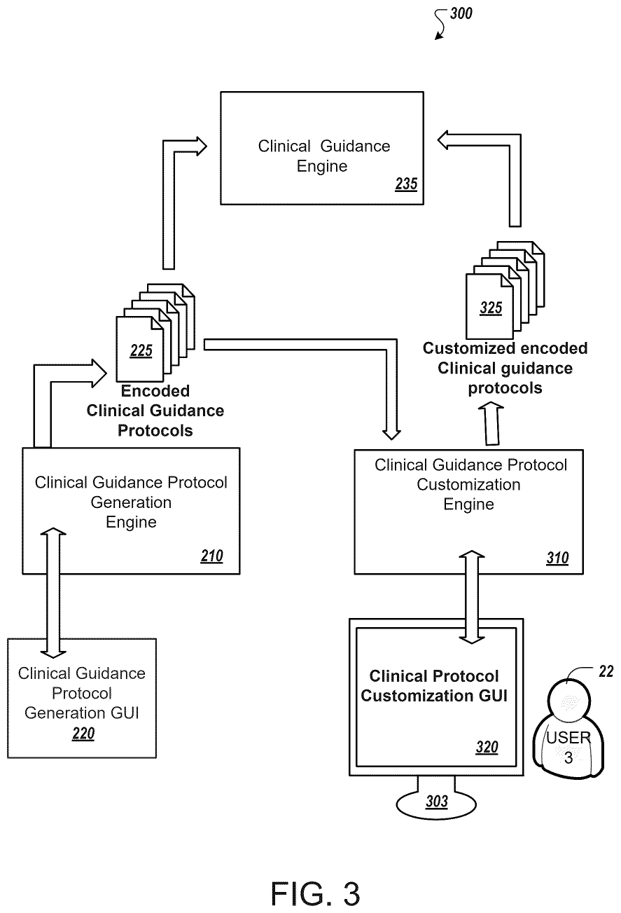

Referring to FIG. 3, an example of a system for generating and customizing an encoded clinical guidance protocol is shown. A quantity of each component in FIG. 3 is an example only and other quantities of each, or any, component could be used. The system 300 includes a clinical guidance protocol customization engine 310 in addition to the components of the system 200 in FIG. 2A. The clinical guidance protocol customization engine 310 includes hardware logic and/or software logic (e.g., as provided by a processor, a memory, and associated circuitry) that enables the clinical guidance protocol customization engine 310 to implement a clinical guidance protocol customization GUI 320 at a display device 303. The clinical guidance protocol customization engine 310 may retrieve the encoded clinical guidance protocol(s) 225 generated by the clinical guidance protocol generation engine 210. For example, the customization engine 310 may retrieve the protocol 225 from the platform resource manager 240, the local resource manager 260, and/or the agency resource manager 280. In an implementation, a third party (e.g., a third user 22), such as a medical director for a particular ambulance agency or hospital may customize an encoded clinical guidance protocol to create customized encoded clinical guidance protocol(s) 325. In some examples, the third user 22 may be a same user as the first user 20 and/or the second user 21. In some examples, the display device 303 may be a same device as one or more of the device 203 and the device 255.

The customization engine 310 may enable adjustments and/or modifications of various specific criteria of the generated protocol 225, the addition or removal of protocol steps, and/or combining multiple clinical guidance protocols 225. The customization may account for specific protocol preferences of an agency, agency system, hospital, or hospital system. However, the customization engine 310 may work within a pre-existing framework provided by a previously generated clinical guidance protocol 225. In contrast, the generation engine 210 creates the encoded clinical guidance protocol that the customization engine 310 may modify to customize. In an implementation, the customization engine 310 may provide the customized encoded clinical guidance protocol(s) 325 to one or more of the platform resource manager 240, the local resource manager 260, the agency resource manager 280, and/or the clinical guidance engine 235. The clinical guidance engine 235 may provide clinical guidance for a caregiver at least in part at the clinical guidance UI 250 using the customized encoded clinical guidance protocol(s) 325, the encoded clinical guidance protocols 225, or a combination thereof.

Referring to FIG. 4A, examples of medical devices and patient interface devices are shown. A quantity of each component in FIG. 4A is an example only and other quantities of each, or any, component could be used. The medical devices 265 may include a ventilation system 480, a patient monitor/defibrillator 482, and/or a trauma kit 484. The patient interface devices 450 may couple to the patient 495 and may include one or more sensors, intervention or treatment delivery devices and/or medical supplies. For example, the patient interface devices 450 may include one or more of a compression sensor 451 (e.g., a motion and/or force sensor configured to detect chest compressions), drug delivery device 452, an SpO2 sensor 454, a CO2 sensor 456, a non-invasive blood pressure (NIBP) sensor 458, electrodes 460, an airway pressure sensor 462, a pneumotachometer 464, a temperature sensor 466, an invasive blood pressure (IBP) sensor 468, an intubation tube 470, a mask 472, a nasal cannula 474, and/or a spirometer 476. The electrodes 460 may sense the heart rate and the electrical activity of the victim's heart and/or may deliver electrotherapy (e.g., defibrillation shock and/or pacing) to the patient 495. The electrodes 460 may include 12-lead electrodes.

Referring to FIG. 4B, an example of a communications configuration for the CGPG system is shown. A quantity of each component in FIG. 4B is an example only and other quantities of each, or any, component could be used. The system 402 includes the display 255 that provides at least a portion of the clinical guidance UI 250. The system 402 further includes the clinical guidance engine 235 and the encoded clinical guidance protocol 225. In order to implement the encoded clinical guidance protocol 225, the clinical guidance engine 235 may exchange information with the caregiver 490 (e.g., the second user 21) and/or the medical devices 265. The medical devices 265 may be configured to couple to the patient 495 via the patient interface devices 450. In various examples, the clinical guidance engine 235 may be communicatively coupled to various patient charting tools 455. The patient charting tools 455 may include one or more of an electronic patient care report (ePCR) application, an ePCR server, an electronic medical record (EMR) application, or an EMR server. The ePCR application or server may provide charting tools and records for an EMS agency and the EMR application or server may provide charting tools and records for a hospital, physician, and/or another medical practitioner. The ePCR itself may be associated with and include information for a single encounter for a single patient (e.g., only one encounter with EMS) whereas the EMR may be associated multiple encounters with multiple practitioners (e.g., medical visits over a period of weeks, months, or years) for a single patient. In an implementation, the clinical guidance engine 235 may determine whether to prompt a user for input at the clinical guidance UI 250 based on the availability of the patient charting tools 455. For example, a protocol may need the age of the patient to make a decision and the clinical guidance engine 235 determine if this value has been previously entered at the clinical guidance UI for the current encounter with the patient or may query the patient charting tools 455 for this information. In an implementation, the clinical guidance engine 235 may generate a UI control to prompt a user for entry of the required information, here the age, only if such information is otherwise unavailable (e.g., has not been previously entered at the clinical guidance UI 250 and/or is not available from the patient charting tools 455). As described below, the implemented protocol may include a protocol block that the clinical guidance engine 235 may implement to determine if a charting tool is available or if a manual entry is needed at the clinical guidance UI 250.

The clinical guidance engine 235 may receive various inputs and generate and send various outputs. The clinical guidance engine 235 may receive physiological data 420 from the medical devices 265 (e.g., as collected from the patient via the patient interface devices 450). The clinical guidance engine 235 may provide medical device instructions 425 to the medical devices 265. Furthermore, the clinical guidance engine 235 may receive caregiver input 430 and may provide caregiver guidance 435. The communication of the caregiver guidance 435 and the caregiver input 430 with the clinical guidance engine 235 may occur via the clinical guidance UI. The clinical guidance UI 250 may be provided by one or more devices and may not be limited to a display 385. For example, in addition to and/or in lieu of the display 385, the user interface device 255 may be and/or may include one or more of an audio device (e.g., a combination of speaker and microphone), a haptic device, and/or a virtual reality and/or an augmented reality display device and may provide the clinical guidance UI 250.

In addition to the physiological data 420, the clinical guidance engine 235 may receive medical device status information, and/or requests for information from the medical devices 265. In an implementation, the clinical guidance engine 235 may set prioritization schemes for inputs from the medical devices 265. The clinical guidance engine 235 may receive a particular type of input from multiple sources. For example, two or more of the patient monitor/defibrillator 482, the ventilation system 480, and the SpO2 sensor 454 equipped with a Bluetooth® sensor may provide an SpO2 measurement to the clinical guidance engine 235. Given these two or more inputs, the protocol 225 and/or a configuration of the clinical guidance engine 235 may pre-determine a prioritization amongst these sources. This prioritization may be customized based on the protocol 225 or the implementation of the clinical guidance 235. Additionally, or alternatively, the prioritization may account for real-time relative signal quality between signal sources and/or risk mitigation. In an implementation, the clinical guidance engine 235 may select from amongst these signals based on a signal quality assessment and select the highest quality signal (e.g., a high signal/noise ratio, few gaps in the signal, etc.). The clinical guidance engine 235 may also prioritize a physiological parameter value based on a signal from a device under closed-loop control rather than from another source. For example, if the clinical guidance engine 235 receives an SpO2 signal from both of a patient monitor and a ventilation system and is running the ventilation system under closed loop control, then the clinical guidance engine 235 may prioritize the SpO2 signal originating from the ventilation system. One example of an advantage to such a prioritization is that if there is loss of signal connectivity with the patient monitor, this loss would not affect the control of the ventilation system.

The medical device instructions 425 may include instructions to perform a particular process or procedure, information for recordation and/or display at the medical device(s), instructions to record and/or display the information, requests for medical data, etc. The requests for medical data may originate from generated protocol 225 implemented by the clinical guidance engine 235 and/or may originate from the clinical guidance engine 235 as a resource provided by the clinical guidance engine 235 in support of an implemented protocol. For example, a particular protocol block in a generated protocol 225 may require a value of a particular physiological parameter. As an example, the particular physiological parameter may be a patient's temperature. In an implementation, the clinical guidance engine 235 may recognize this request for a physiological parameter and determine if an appropriate sensor is communicatively coupled (e.g., by evaluating the network 499 shown in FIG. 4C) and/or if a value of the physiological parameter is available from a patient charting tool (e.g., through a query of the patient charting tool 455 in FIG. 4A). If so, the clinical guidance engine 235 may automatically obtain the value from the sensor or from the patient record. However, if this value is not available from either of these resources, then the clinical guidance engine 235 may generate a UI control at the clinical guidance UI 250 to either prompt the second user 21 to enter the value and/or to connect the appropriate sensor. For the example of temperature, the clinical guidance UI 250 may prompt the user to enter in a measured temperature, may provide guidance on how to obtain a temperature, and/or or may prompt the user to use a temperature sensor that can communicatively couple to the clinical guidance engine 235. As described below, the implemented protocol may include a protocol block that the clinical guidance engine 235 may implement to determine if a sensor is coupled or if a manual entry is needed at the clinical guidance UI 250.

In an implementation, the instructions to the medical device to perform a particular process or procedure may cause the medical device(s) to automatically perform the particular process or procedure. In such an implementation, the instructions may function as a control signal. Instructions 425 sent to the medical device(s) may include instructions to perform an intervention, parameters of the intervention, and/or instructions to record information about an intervention.

Aspects of the present disclosure are also directed to allowing a user, via inputs at the clinical guidance UI 250, to supply inputs to the medical devices 265. During treatment of a critically ill patient, rescuers in the immediate vicinity of a patient are often consumed with tending to the medical needs of the patient, whether that includes administering electric shock or ventilation via the medical treatment device, administering chest compressions, administering ventilation, or treating wounds. Additionally, user input interfaces (e.g., keypads and other buttons for inputting information) that are local to the medical treatment device can be cumbersome to operate in time-critical situations. Instead, in some examples, users of a more conveniently located clinical guidance UI 250 may control one or more functional operations and/or provide one or more inputs. For example, the clinical guidance UI 250 may be a touchscreen or an audio input device or a virtual screen at a heads-up device. In some scenarios, the caregiver providing direct care to the patient may also operate the clinical guidance UI 250. In other scenarios, a caregiver that is providing intermittent care and/or assistance to another caregiver and not providing direct care to the patient for some period of time may operate the clinical guidance UI 250. In some examples, users of the clinical guidance UI 250 can input patient information, record event markers, initiate 12-lead ECG analyses, or record a device snapshot. Therefore, allowing a user to provide instructions to activate one or more operations of the medical treatment device via the clinical guidance UI 250 provides enhanced technical flexibility that is not available when operating locally at the medical treatment device by allowing supervisors or other personnel at the scene of a medical event to observe, in real-time, how the medical event is progressing without having to hover over the treatment area, which may impede patient care.

In response to receiving user inputs at the clinical guidance UI 250 associated one of the control operations at the medical treatment device, the clinical guidance engine 235, in some implementations, transmits an instruction signal to cause the respective operation to occur at the medical treatment device. In some examples, instruction signals sent from the clinical guidance engine 235 to the medical treatment device can instruct the medical treatment device to update patient information, treatment information, or diagnostic information for the medical event. In response to receiving the respective signal, the medical treatment device performs the respective operation associated with the instruction signal, which may include storing provided information (e.g., transmitting patient information for updating at the medical treatment device) or recording an event marker (e.g., transmitting a treatment/event marker for the medical treatment device to record in the patient care record) or initiating a snapshot (e.g., transmitting an instruction signal for the medical treatment device to initiate a snapshot of ECG associated with the time of the instruction input) or activating an analysis feature (e.g., instruction signal for the medical treatment device to perform a 12-lead analysis) at the medical treatment device. In some embodiments, the instruction signals can also include control signals for causing the medical treatment device (a defibrillator) to initiate electrotherapy or apply another therapeutic treatment. When the medical treatment device is the ventilation system 480, the instruction signals can include control signals to administer the positive pressure ventilation to the patient. In some examples, upon initiation and/or completion of the respective operation, the medical treatment device transmits a notification signal to the clinical guidance engine 235. In response to receiving the notification signal from the medical treatment device, the clinical guidance engine 235 can cause display of a notification message at the clinical guidance UI 250 that the respective action is being performed; and then a subsequent notification signal from the medical treatment device for the companion device to display a notification message that the respective action has been performed.

In some examples, the medical device instructions 425 may enable closed loop control of a particular medical device by the clinical guidance engine 235. The term closed loop control, as used herein, may refer to control of one or more treatment related or patient related parameters, such as with relatively little or no required user action, participation or intervention, and can include reference to, but is not limited to reference to, fully automated or fully automatically regulated control. Closed loop control may include, for example, device facilitated or algorithmically facilitated tracking, control and adjustment of one or more parameters, which may or may not include user involvement or participation. Where user involvement or participation is included, it may include, for example, confirming a suggested or recommended medical device setting change or configuration, deciding on implementing a course of action, selecting one of several suggested courses of action, responding to a presented alert or alarm, or other decisions, choices or actions. User involvement or participation could also include, for example, setting or changing a parameter, where a closed loop control algorithm proceeds from there, initially according to the user-set or user-changed parameter setting. In various embodiments, if there is user involvement, it may be, for example, among other things, in whole or in part user-initiated, or in whole or in part prompted, suggested, recommended or required.

In some embodiments, closed loop control may be utilized but may be subject to manual adjustment or override by the user. For example, in some embodiments, although treatment parameters may be algorithmically and automatically controlled, a user may be able to intervene and manually change the treatment parameter setting. In some embodiments, following any manual adjustments, closed loop control of the treatment parameters may resume from that point, at least until any further manual adjustments are made.

Referring to FIG. 4C, examples of medical device communications are shown. A quantity of each component in FIG. 4C is an example only and other quantities of each, or any, component could be used. In an implementation, one or more of the ventilation system 480, the patient monitor/defibrillator 482, and the trauma kit 484 may communicatively couple to one another as part of a network 499. Additionally, or alternatively, one or more of the ventilation system 480, the patient monitor/defibrillator 482, and the trauma kit 484 may communicatively couple to one or more of the user interface device 255 and the clinical guidance engine 235. As such, the various devices and engine shown in FIG. 4C may share resources such as user interface, data management, external communication, processing of diagnostic/treatment algorithms, and/or processing and/or providing of various physiological signals. The communicative couplings in FIG. 4C may be wired and/or wireless communications links. The various devices and engine shown in FIG. 4C may include and/or be coupled to a network interface configured to couple to the network 499 which may be a local network, a wide area network, or a combination thereof. For example, the network interface may enable a short-range wireless network (e.g., a personal area connection (PAN) connection, such as a BLUETOOTH connection, or a local area network (LAN) connection, such as a WIFI connection) and/or a long-range wireless connection (e.g., a wide area network (WAN) connection, such as a Code-division Multiple Access (CDMA) connection or Global System for Mobile Communication (GSM) connection). The network may include a cellular network and/or a computer network such as the Internet. In an implementation, the devices and engine may be pre-configured to be communicatively coupled so as to streamline wireless communication pairing without having to undergo a time-consuming inquiry and response negotiation for a secure connection to be established. The various devices and engine shown in FIG. 4C may be local devices as they are located in proximity to the caregiver 490 (e.g., user 21) and the patient 495. In contrast, the cloud server 270, the platform 202, and the clinical guidance protocol generation display device 203 are all remote devices because they are located remotely from all of the devices and engine shown in FIG. 4C.

In an implementation, the network 499 may include an edge server 498. The edge server 498 may be a computing device configured to execute processor intensive operations that are sometimes involved when executing operations of the clinical guidance engine 235. Some implementations of the edge server 498 include, for example, one or more GPUs that are capable of efficiently executing matrix operations and substantial cache or other high-speed memory to service the GPUs. In some examples, the edge server 498 is a separate, ruggedized physical device that travels with EMS personnel in the field. In some examples, the edge server 498 is incorporated into other EMS field equipment such as a medical device and/or may be located in the EMS vehicle and/or may be located within a carrying case for a medical device. In an implementation, the user interface device 255 may provide the edge server if the processing capability of this device is sufficient to provide computing services associated with an external edge server. The edge server moves more computing capability into the local environment so that computation intensive clinical guidance can run accurately and efficiently even in the absence of a connection with the remote cloud server 270 and the platform 202. In an implementation, the edge server 498 may include the clinical guidance engine 235 and the local resource manager 260.

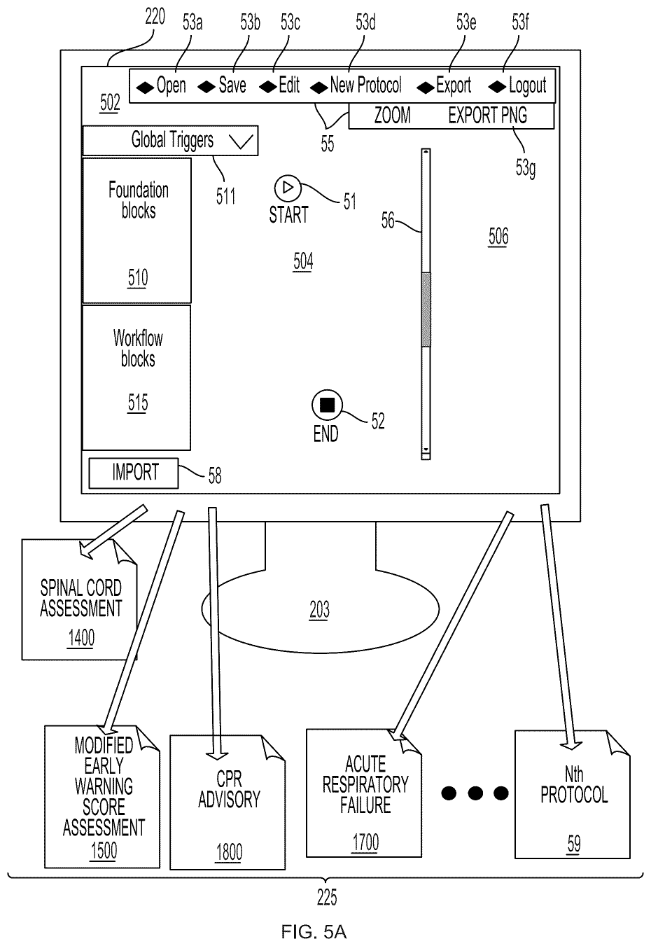

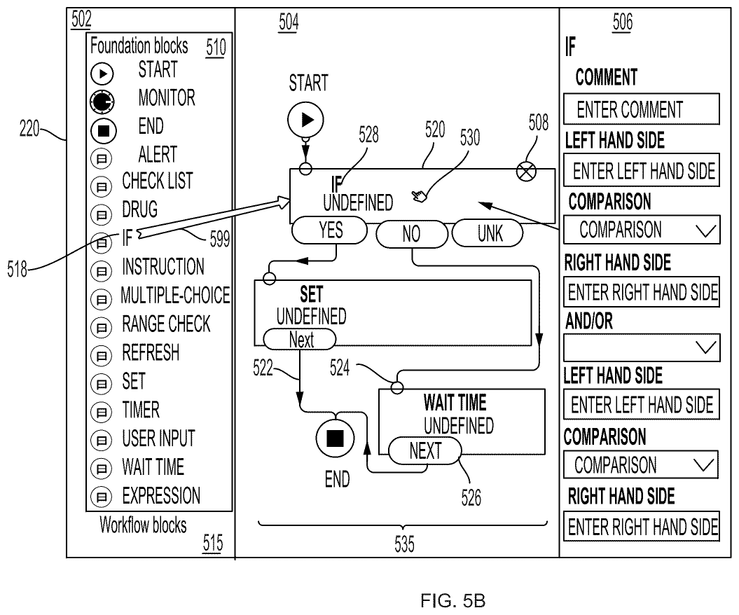

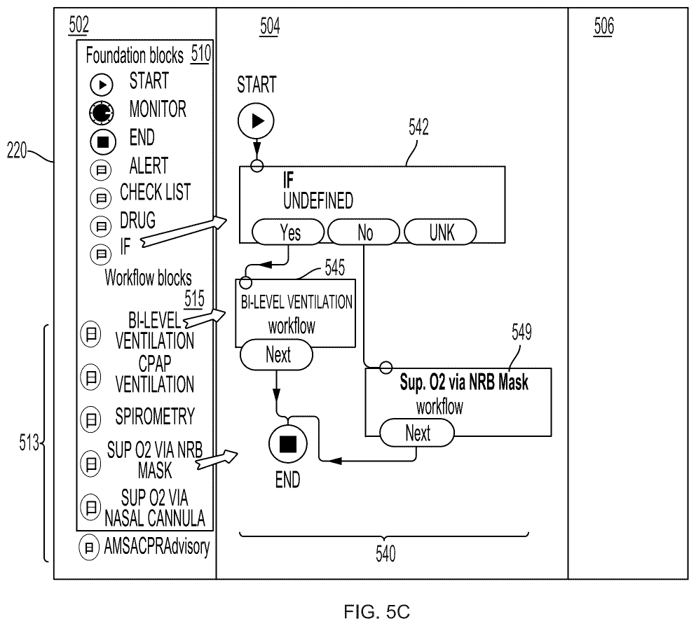

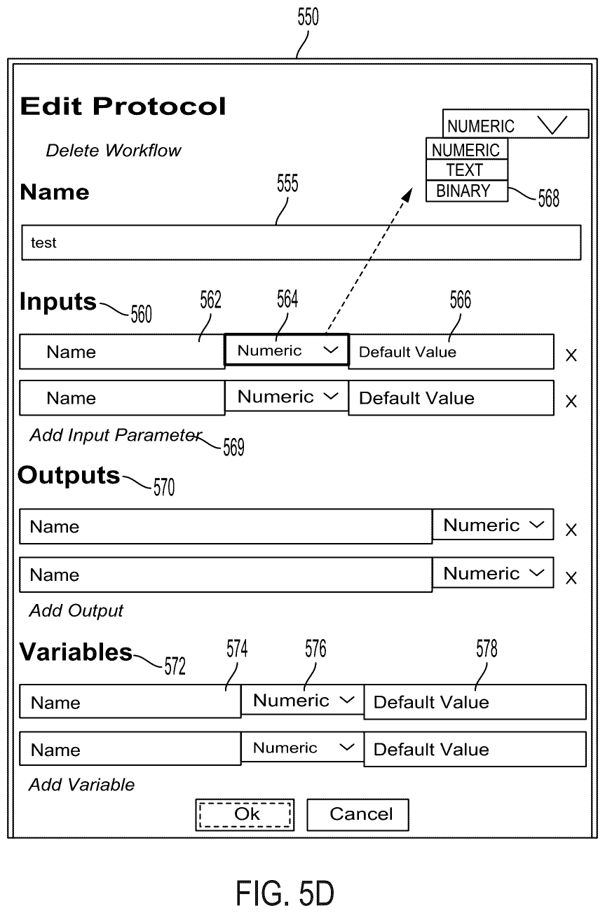

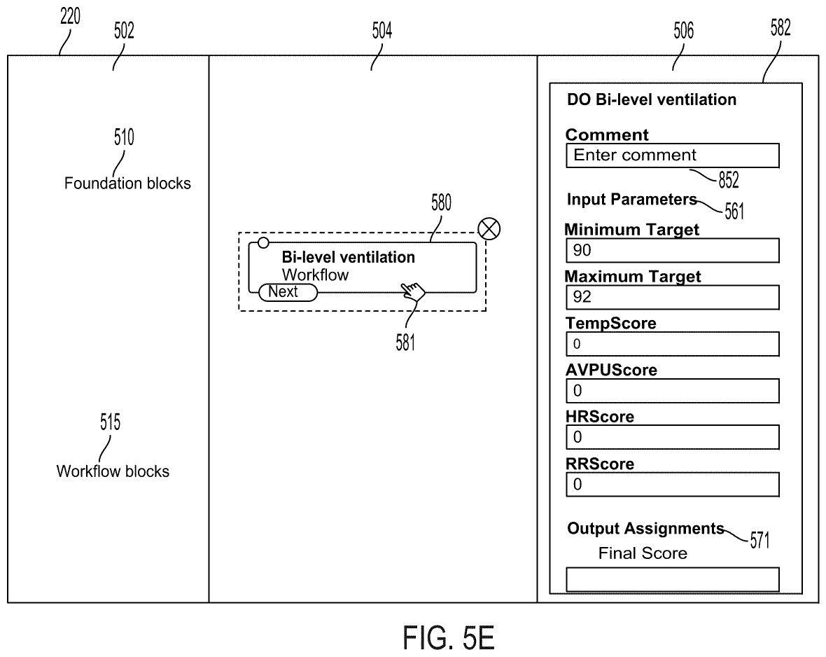

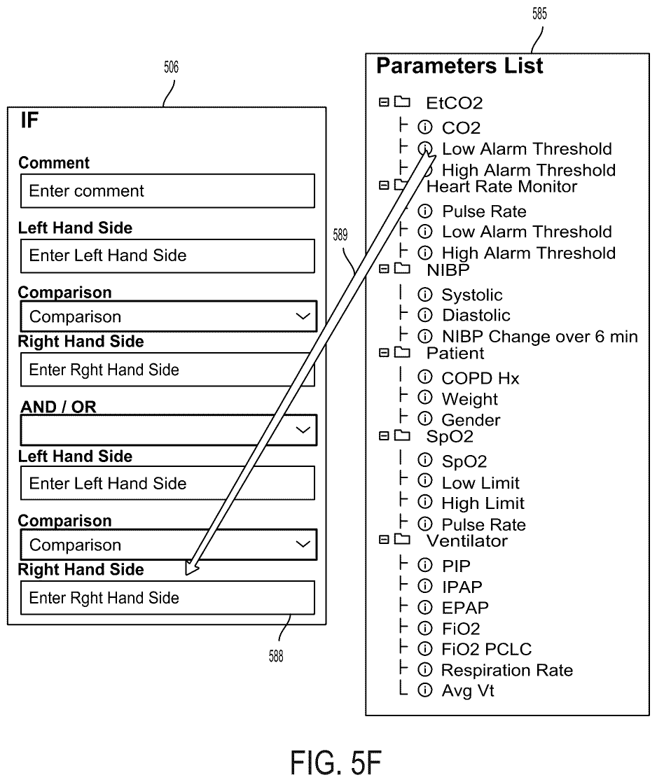

Referring to FIG. 5A, an example of a basic layout for a CGPG graphical user interface (GUI) is shown. A quantity of each component in FIG. 5A is an example only and other quantities of each, or any, component could be used. The CGPG engine 210 may provide the CGPG GUI 220 at a display device 203, as shown for example in FIG. 2A. The CGPG GUI is described at an overview level here and in more detail below.

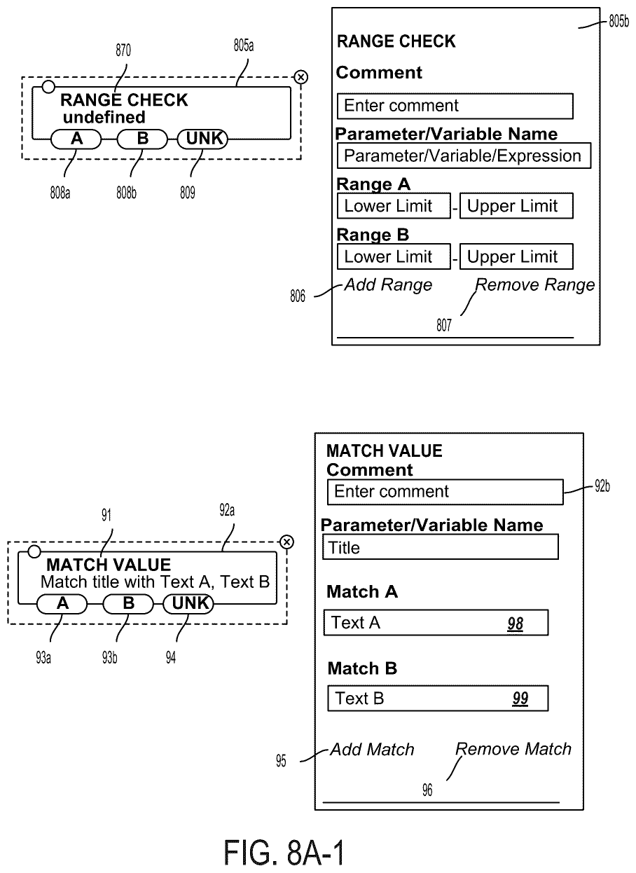

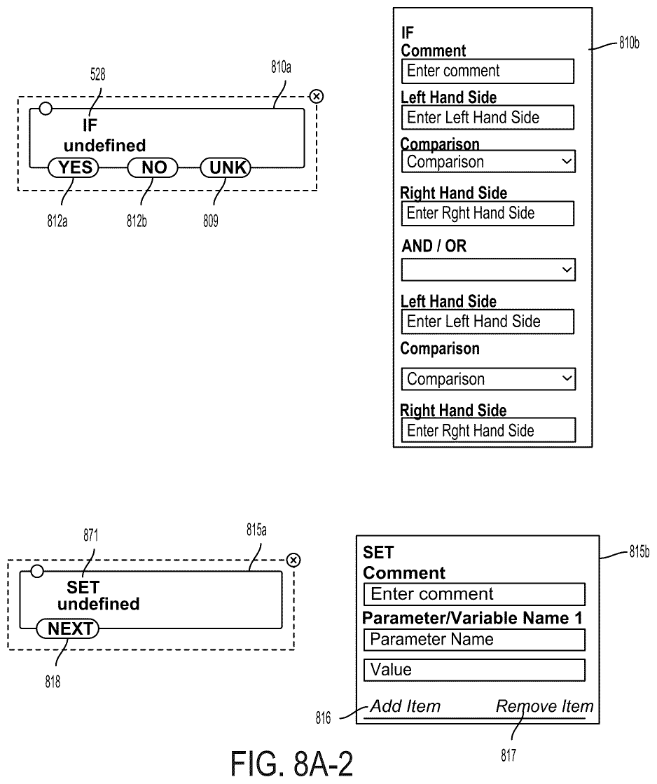

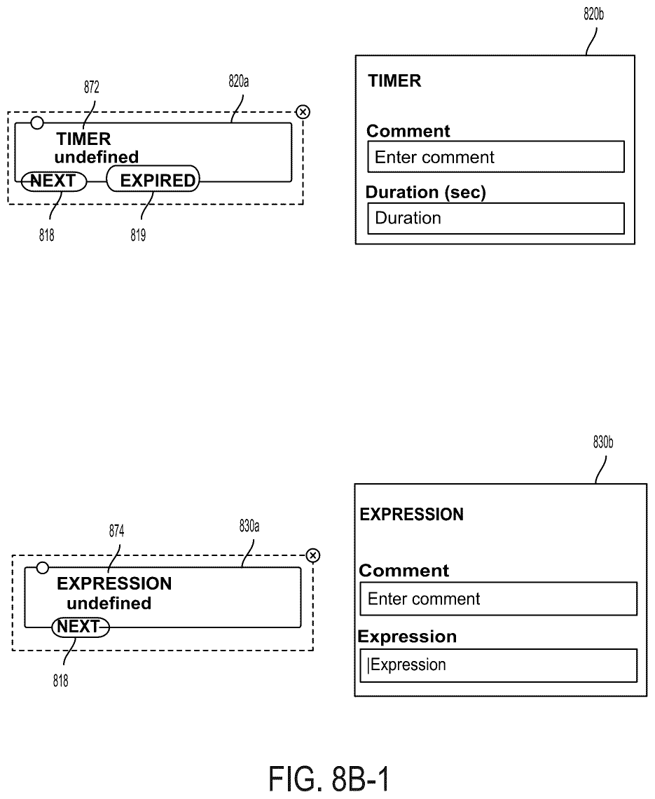

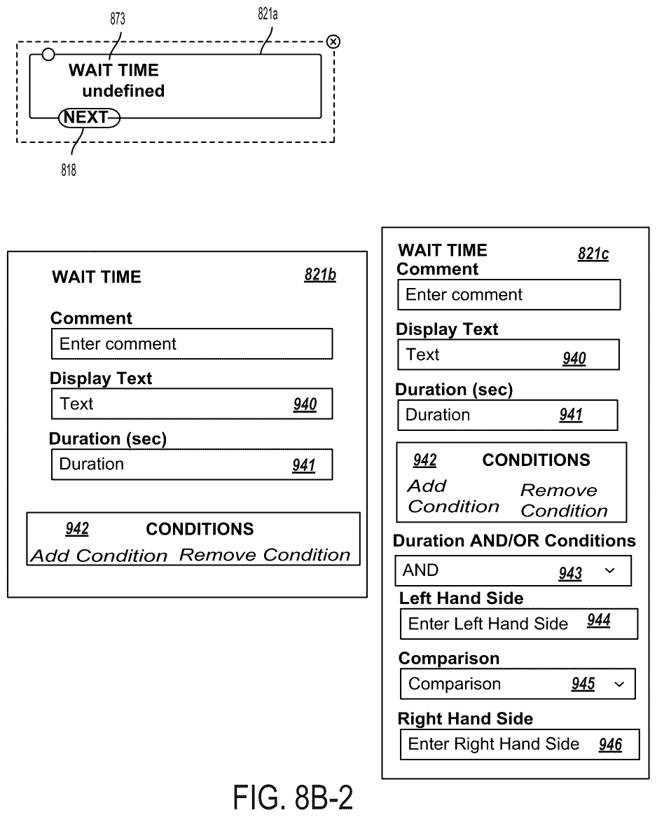

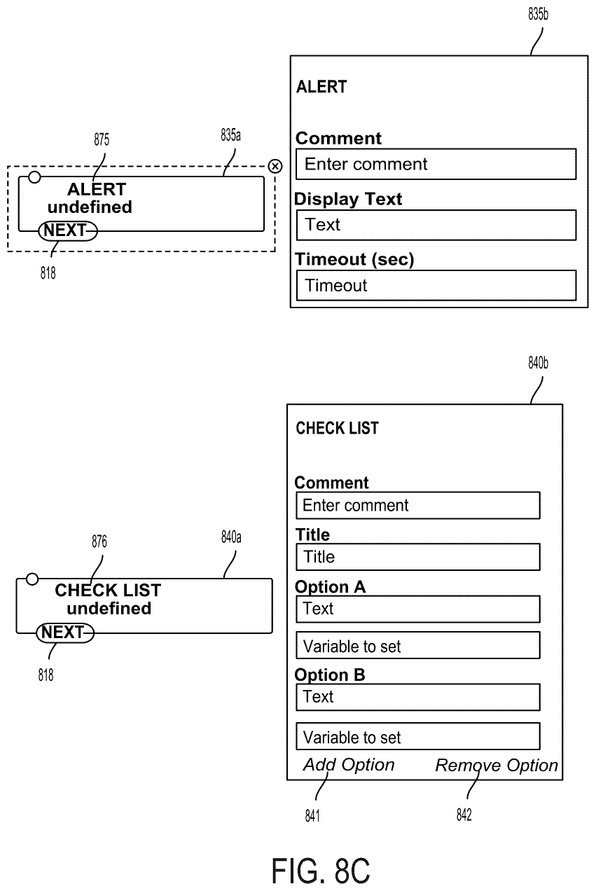

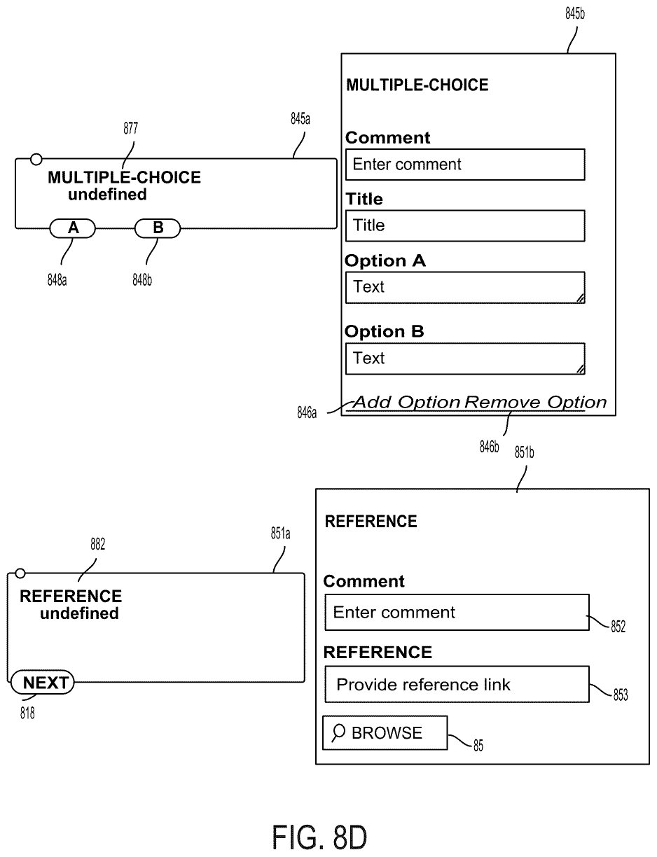

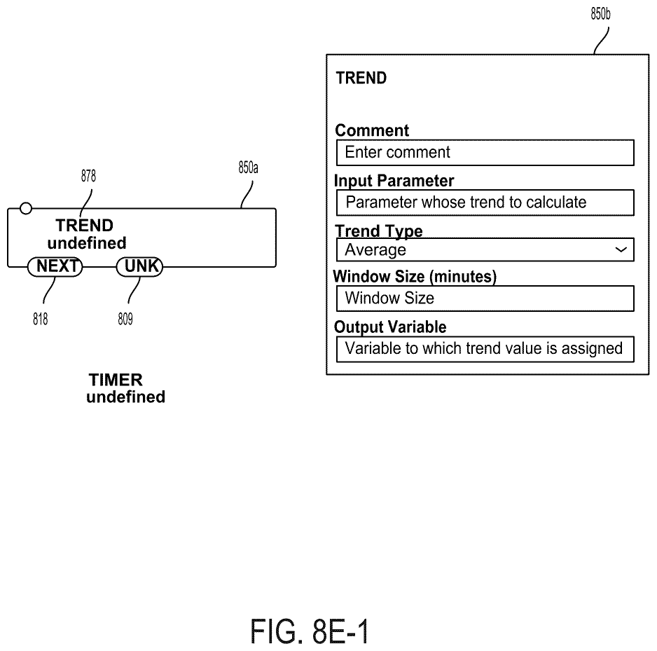

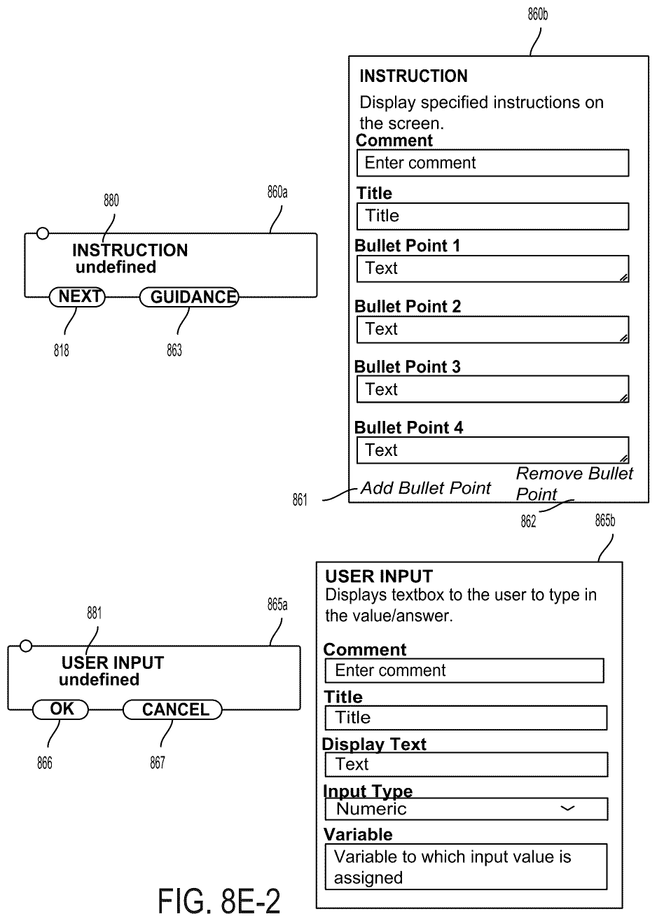

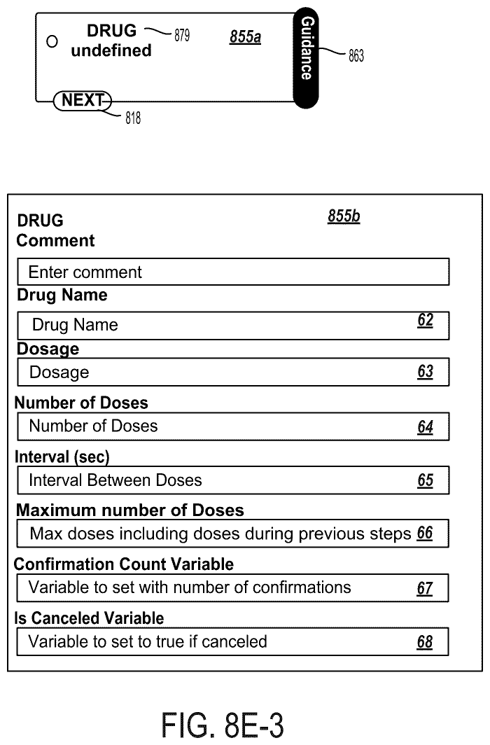

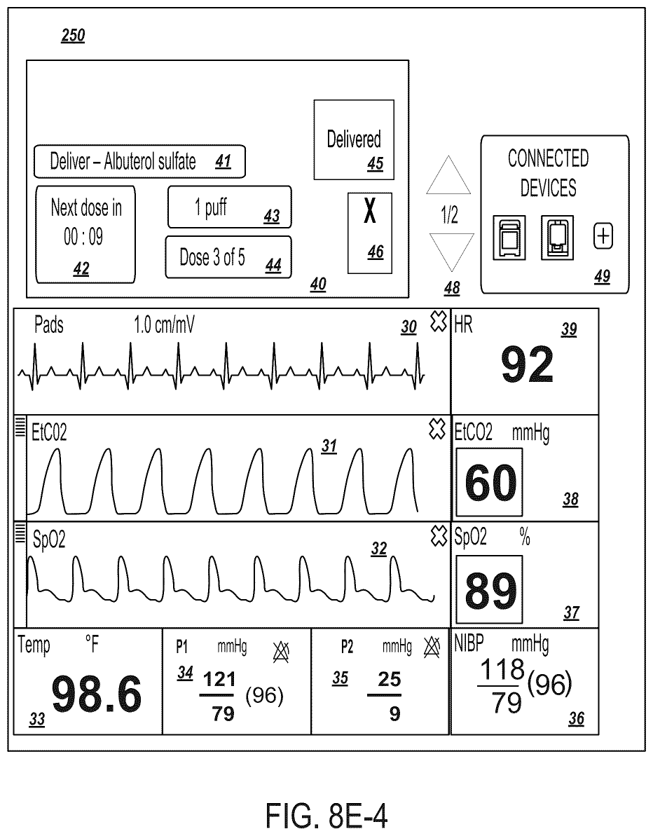

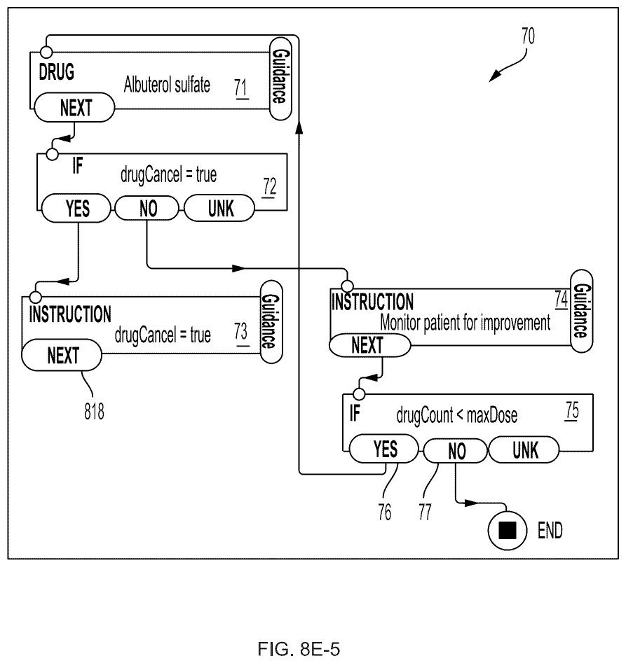

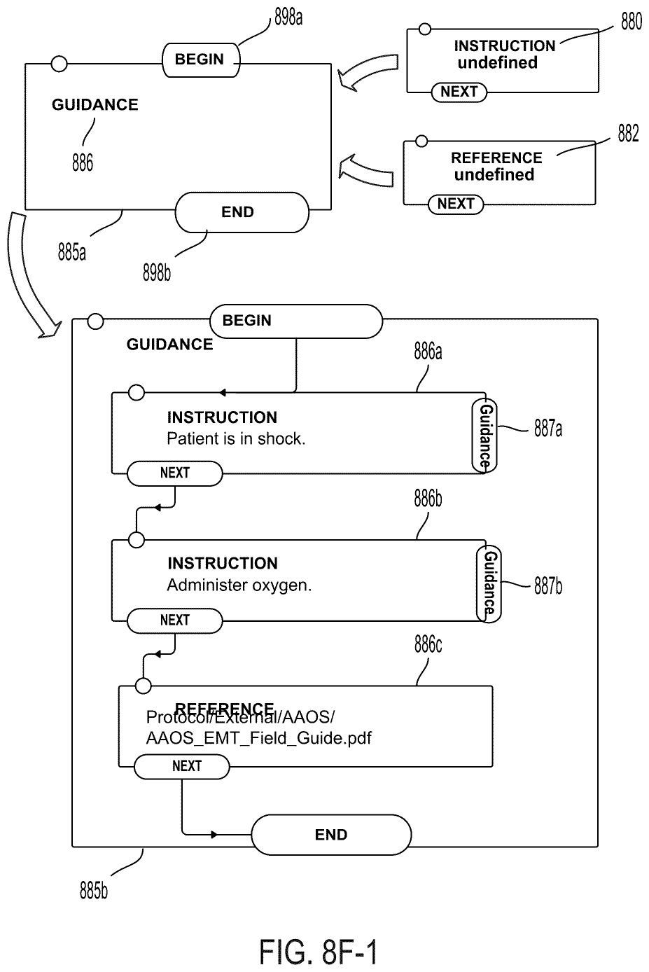

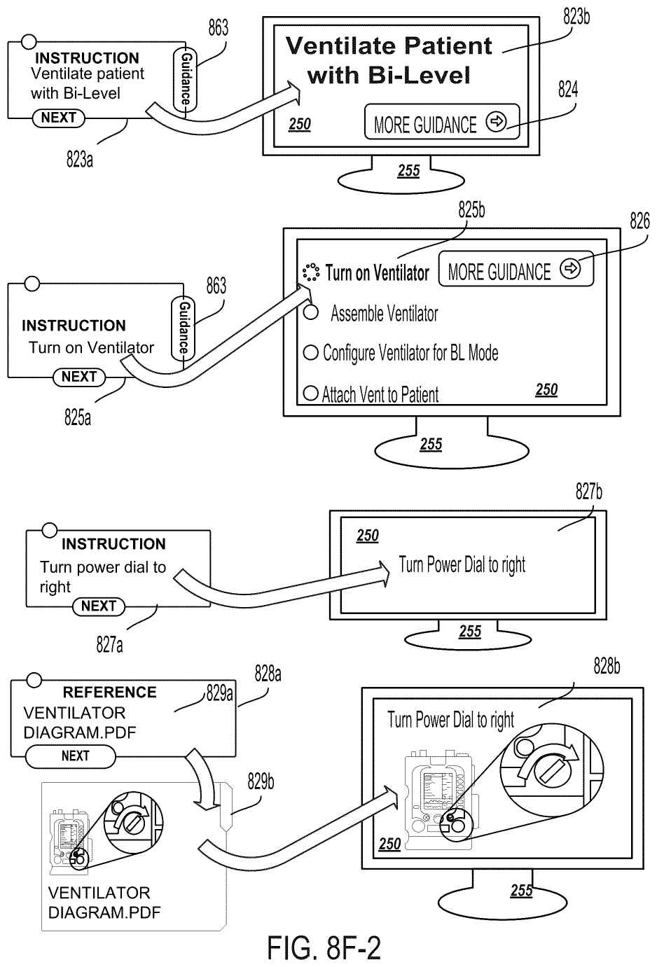

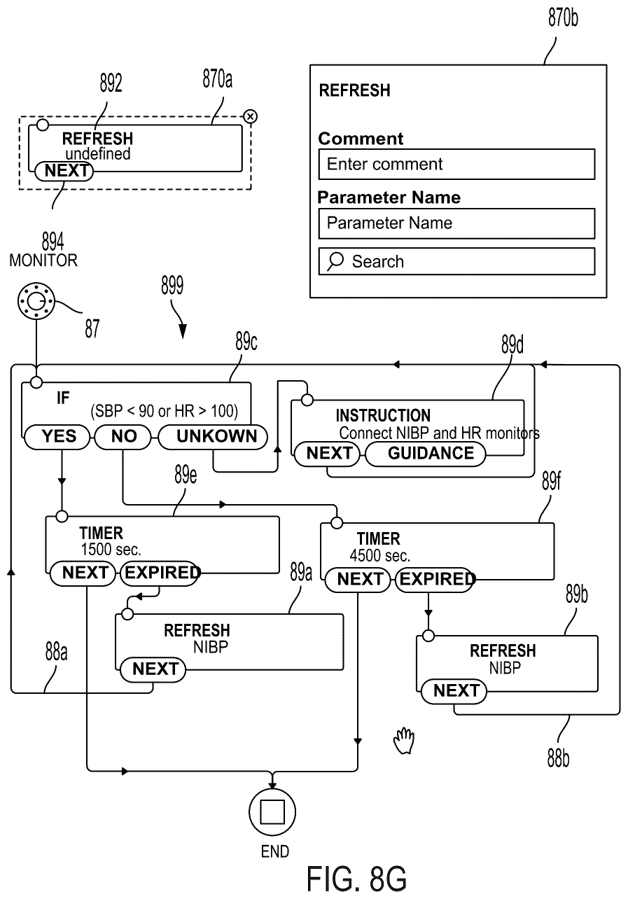

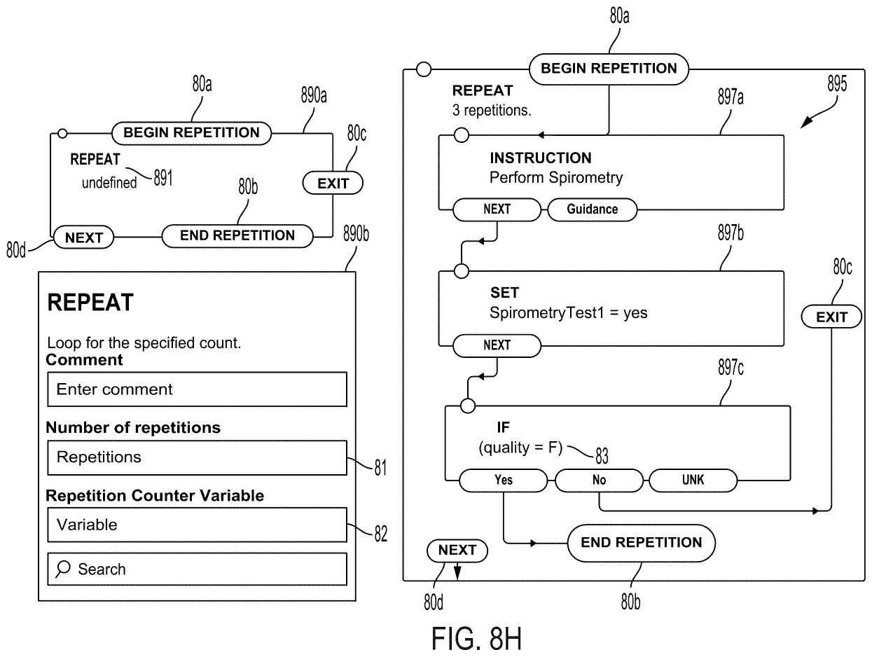

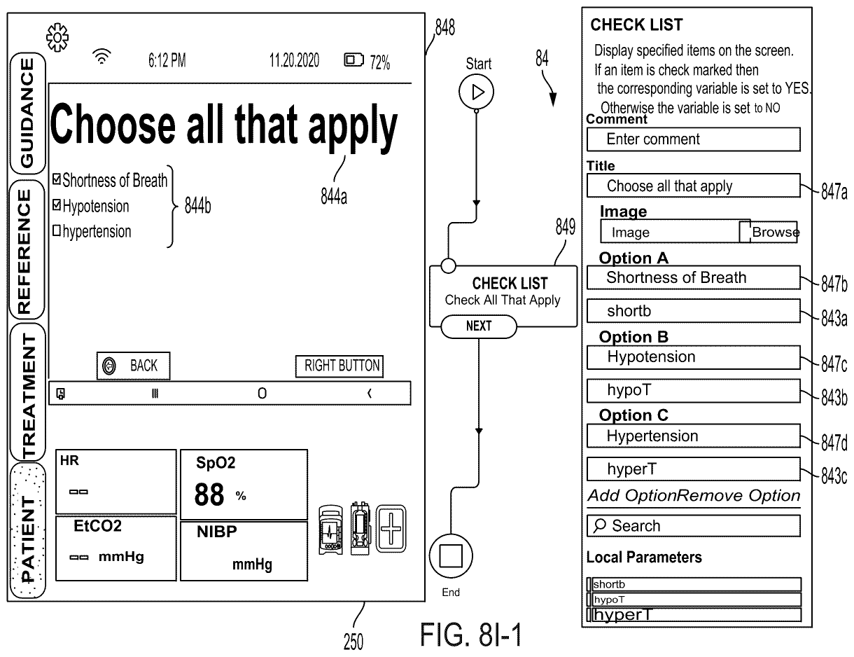

The CGPG GUI 220 may be configured to provide a protocol block selection menu 502, a graphic protocol display area 504, and a user specification window 506. The protocol block selection menu 502 includes a foundation block menu 510 and a workflow block menu 515. The menus 510 and 515 include blocks that are available for selection by a user of the CGPG GUI 220. For example, the user may select one or a plurality of protocol blocks from the selection menu 502 by clicking on a block and dragging it into the graphic protocol display area 504. The graphic protocol display area 504 may be pre-populated with a start block 51 and a stop block 52. The user of the CGPG GUI 220 may build the protocol in the display area 504 beginning at the start block 51 and ending at the stop block 52. The protocol blocks as selected from the menu may be unspecified in nature and when a protocol block is highlighted in the graphic protocol display area 504, the CGPG GUI 220 may provide fillable fields corresponding to the selected protocol block in the user specification window 506. These fillable fields enable a user to specify various aspects and features of the selected protocol block. The CGPG GUI 220 may further include file management and screen controls 55, a scrolling control 56 for the user specification window 506, and an import control 58. The file management and screen controls 55 may include a control to open 53a a previously saved protocol, a control to locally save 53b a protocol in progress or a generated protocol at the CGPG engine 210, a control to edit 53c a protocol opened with the control 53a, a control to generate a new protocol 53d, a control to export 53e a generated protocol to a resource manager, a control to logout 53f of a protocol generation session, and a control 53g to export an image of the graphical representation of the clinical data protocol. For example, this image may be a portable network graphic (PNG) image. The control 53g may enable a user of the CGPG GUI 220 to view and share an image of the generated protocol steps and sequencing away from the CGPG GUI 220. This may be helpful in reviewing the protocol for content and accuracy, soliciting clinical opinions from other clinicians or from users, etc. This review does not require any review, understanding, or knowledge of the processor-readable or processor-executable code in the encoded clinical guidance protocol 225. The import control 58 may enable the user to import previously generated protocols and display these at the CGPG GUI 220 in the workflow block menu 515. Some examples of encoded clinical guidance protocols 225 that may be generated at the CGPG GUI 220 include, but are not limited to, a spinal cord assessment workflow 1400, a modified early warning score assessment workflow 1500, an acute respiratory failure workflow 1700, and a CPR advisory protocol 1800. These examples are described in more detail below. Any number of protocols may be generated at the CGPG GUI 220 as indicated by Nth protocol 59. The CGPG GUI 220 may further include a protocol block category selection menu (e.g., the drop-down menu 511). This menu enables the user to limit the foundation block and/or workflow block menus to particular categories of protocol blocks where the particular categories may have unique attributes in regard to how these blocks function when implemented by the clinical guidance engine 235. One example is initiation blocks, or background triggers, as discussed in detail below following the description of FIG. 8G.

Referring to FIG. 5B, an example of a clinical guidance protocol generation (CGPG) GUI is shown. A quantity of each component in FIG. 5B is an example only and other quantities of each, or any, component could be used. The CGPG engine 210 may provide the CGPG GUI 220 at a display device 203, as shown for example in FIG. 2A.

As described above in regard to FIG. 5A, the CGPG GUI 220 may be configured to provide a protocol block selection menu 502, a graphic protocol display area 504, and a user specification window 506. The protocol block selection menu 502 includes a foundation block menu 510 and a workflow block menu 515. A protocol generated at the CGPG GUI 220 is specified by a sequence of steps performed by the caregiver (e.g., the second user 21), a medical device 265, the clinical guidance engine 235, or a combination thereof. Each step in the generated protocol is encapsulated by a block provided at the CGPG GUI 220. The blocks in these menus are available for selection by a user of the CGPG GUI 220. For example, the user may select one or a plurality of protocol blocks from the selection menu 502. The user may select the protocol blocks via a drag and drop operation 599 at the CGPG GUI 220. The selection of a protocol block causes the CGPG GUI 220 to display a graphic representation 520 of the protocol block 518 in the graphic protocol display area 504.

Depending on the form of the encoded clinical guidance protocol 225, the foundation blocks correspond to data objects in a data interchange format file, a set of processor-executable instructions such as web application code, a section of a processor-executable image file, or other processor-executable code. The foundation blocks define certain tasks that are implemented by the clinical guidance engine 235. The foundation blocks may be sequenced at the CGPG GUI 220 to define a protocol. For example, as shown in FIG. 5B, a sequence of foundation blocks from the menu 510 form an encoded protocol 535.

The workflow blocks 513 in the menu 515 may function as stand-alone protocols or may be sub-protocols that are sequenced with foundation blocks within a larger stand-alone protocol. The larger stand-alone protocol is an envelope protocol that may include multiple workflow blocks. The envelope protocol may also be described as an envelope workflow that includes sub-sections in the form of smaller workflow blocks. A workflow block 513 may be triggered in a variety of ways. For example, once a workflow block 513 is defined and available in the menu 515, it may be incorporated in a higher-level envelope protocol. The envelope protocol may specify when or under what conditions that higher level protocol triggers a built-in workflow. For example, for an acute respiratory failure (ARF) workflow (e.g., the workflow shown in FIG. 17A), a condition evaluation block that checks EtCO2 and SpO2 values to enter the ARF protocol may trigger the ARF workflow block. Alternatively, the clinical guidance UI 250 may provide a touchscreen control and a manual activation of that control by the user (e.g., the second user 21) may trigger implementation of a particular workflow. For example, a pediatric or geriatric workflow may require entry of a patient age in order to trigger implementation. An ARF workflow may require entry of a particular SpO2 value by the user of the clinical guidance UI 250.

The workflow blocks 513 may include built-in workflow blocks and imported workflow blocks. The built-in workflow blocks may include foundation blocks and/or may include protocol blocks that are more complex in nature than the foundation block. As discussed below, for example, in regard to FIG. 5C and FIG. 9, the built-in blocks may include, for example, a bi-level ventilation block, a CPAP ventilation block, a spirometry block, a supplemental oxygen with non-rebreather mask block, a supplemental oxygen with nasal cannula block, an amplitude spectrum analysis (AMSA) CPR advisory block, an acute respiratory failure (ARF) block, etc. These built-in blocks are examples only and not limiting of the disclosure. The built-in blocks may center around procedures for respiratory conditions, cardiac conditions, trauma, sepsis, and other patient conditions.

The previously generated protocols are workflow blocks that may be nested within another clinical guidance protocol. These previously generated protocols may include foundation blocks combined with one or more of built-in blocks and other previously generated protocols. As examples the previously generated clinical guidance protocols may include a spinal cord immobilization workflow block, an acute respiratory failure (ARF) workflow block, or a modified early warning score (MEWS) workflow block. FIGS. 14, 15A-15E, 17A-17B, and 18A-18B provide examples of clinical guidance protocols for spinal cord evaluation, MEWS score evaluation, acute respiratory failure, and AMSACPR advisory that may be generated at the CGPG GUI 220. These protocols, once generated, may be saved and available to embed within another protocol as a workflow block. Alternatively, the provider of the CGPG engine 210 may provide protocols like these as built-in blocks available to a user of the CGPG GUI 220 without initiating the generation of such protocols.

The CGPG engine 210 may provide the built-in workflow blocks at the CGPG GUI 220 and enable a user to merely select the built-in block, embed this block into a higher level, or envelope, protocol to utilize the block without having to determine, manipulate, and order the individual steps within the built-in workflow block that enable the built-in workflow block to perform the desired function. As one example of complexity, the built-in protocol blocks may be specific to a particular item of medical equipment that is necessary to perform the actions of the protocol block. As another example of complexity, the built-in protocol block may include code that defines a sensor prioritization scheme (i.e., to instruct the clinical guidance engine 235 how to pick between sensor values and/or between sensor connection instructions if there is a conflict and/or if two or more sensors are requested concurrently by protocols implemented in parallel). As a further example of complexity, the built-in protocol block may include code that provides the necessary logic for the clinical guidance engine 235 to query the network 499 (as shown in FIG. 4C) to determine if a particular medical device and/or sensor is communicatively coupled and thus able to automatically provide information to the clinical guidance engine 235. Such a block may further include the logic for the clinical guidance engine 235 to implement to generate a UI control at the clinical guidance UI 250 that prompts the user (e.g., the second user 21) to enter the information at the UI 250 because the medical device and/or sensor is not communicatively coupled and therefore unable to automatically provide the necessary information. Similarly, a built-in protocol block may include the logic necessary for the clinical guidance engine 235 to initiate a query to the patient charting tools 455 and provide a manual entry UI control in the instance where these tools 455 are unavailable and/or do not include the desired information. As yet another example of complexity, a built-in block may handle mathematical operations that are more complex than for example simple arithmetic like addition, subtraction, ratios, or multiplication of two or three values. Examples of more complex mathematical operations include integration, differentiation, statistical analysis, filtering, etc. Such operations require more complex programming than the simple arithmetic examples. Some of these more complex operations include, for example but not limiting of the disclosure, peak detection for ECG, evaluations of maxima, minima, ranges, averages, medians, standard deviations for groups of data and/or for waveforms, spectral integrals (e.g., amplitude spectral area determinations for ECG in the treatment of ventricular fibrillation), Fourier transforms, etc. As yet a further example of a built-in block, these may include blocks that require user instructions for specific devices along with guidance and animations. For example, bi-level ventilation and ultrasound measurements may require such specific instructions. Ultrasound may be particularly complex as the built-in protocol block may enable provision of instructions on how to connect the ultrasound probe, how to manipulate the probe to obtain an image, and how to analyze the image to determine a finding. A first user 20 designing a protocol may merely select an ultrasound block, possibly set one or more higher level parameters for the block, and insert the block into an envelope protocol.

Referring to FIG. 5C, an example of a protocol generated with foundation blocks and built-in blocks is shown. A quantity of each component in FIG. 5C is an example only and other quantities of each, or any, component could be used. As exemplified in FIG. 5C, a user of the CGPG GUI 220 may combine workflow blocks (e.g., the bi-level ventilation workflow block 545 and the supplemental oxygen with non-rebreather mask workflow block 549) to generate an encoded protocol 540. The workflow blocks may be the built-in workflow blocks that are provided with the CGPG GUI 220 and/or may be the imported workflow blocks. The protocol 540 also includes the foundation block 542. The foundation block 542 may function as an initiation block for the workflow blocks 545 and 549.

Referring again to FIG. 5B, in addition to protocol blocks, the CGPG GUI 220 enables a user to indicate sequencing links 522 in the graphic protocol display area 504. Each sequencing link 522 connects an input port 524 with an output port 526 to define a progression between protocol blocks. The user specification window 506 provides fillable fields configured to receive entries of user specifications for the protocol blocks. The format of the user specification window 506 is specific to the operator 528 of the protocol block. For example, if the operator 528 is a condition evaluation operator (e.g., the “if” operator shown in FIG. 5B as an example only and not limiting of the disclosure), then the user specification window 506 provides fillable fields according to format specific to the condition evaluation operator. A user selection 530 of a particular protocol block causes the CGPG GUI 220 to display the format of the user specification window 506 corresponding to the operator 528 of the selected protocol block. Additionally, a user selection 530 of the particular protocol block enables the user to drag the block to a specific location within the display area 504 and orient the block relative to other blocks. Each protocol block includes a delete control 508. A user selection of the delete control 508 removes the protocol block from the graphic protocol display area 504.

Referring to FIG. 5D, examples of assigning clinical guidance protocol attributes are shown. A quantity of each component in FIG. 5D is an example only and other quantities of each, or any, component could be used. In an implementation, the CGPG engine 210 may be configured to receive protocol attributes from a user of the CGPG GUI 220. For example, the CGPG GUI 220 may provide an attribute editing window 550. The attribute editing window 550 may be available to edit a protocol that was already fully or partially generated. Alternatively, at the outset of adding a protocol, the attribute editing window 550 may be available to set up the protocol attributes. Protocols added may be saved prior to completion and then added to or rearranged in multiple sessions at the CGPG GUI 220. These attributes may include, for example, a protocol name 555, a protocol input 560, a protocol output 570, or a variable name 572. The CGPG engine 210 may be further configured to save the attributes with the encoded clinical guidance protocol. Further, the CGPG engine 210 may apply the attribute to the encoded clinical guidance protocol such that the attributes provide instructions for invoking the named protocol. The clinical guidance engine 235 may invoke the named protocol as a nested workflow block within a larger, envelope protocol and/or as a workflow block triggered by an initiation block.

In an implementation, the clinical guidance engine may invoke the named protocol subject to at least one attribute provided by the CGPG engine 210. For example, the protocol input 560, the protocol output 570, and protocol variable 572 may provide instructions for invoking the named protocol.

The protocol input 560 and the protocol output 570 are mechanisms for communicating information between the clinical guidance engine 235 and the particular protocol invoked by the clinical guidance engine 235. The clinical guidance engine 235 provides the values of the inputs at the time of invoking the protocol. This enables the clinical guidance engine 235 to customize the behavior of the protocol. The protocol input 560 may be a variable, parameter, condition, or protocol trigger defined in the envelope protocol that is then used by the protocol invoked with that input. The protocol output 570 from a first protocol may provide the protocol input 560 for a second protocol that is implemented subsequent to the first protocol or as a nested protocol within the first protocol. When a workflow block ends, it provides the values for each output to the clinical guidance engine 235. The clinical guidance engine 235 may store these values for use in subsequent blocks of the protocol and/or may assign the output to a particular variable used in a subsequent workflow block of an envelope protocol. Generally, variables are defined and only operate within the context of a particular workflow. However, a protocol input 560 provides a means to pass a variable from one workflow to another.

As an example, a diabetes protocol may require a glucose value in order to operate. The diabetes protocol may include two workflow blocks, a first block configured to implement and analyze a blood chemistry and a second block configured to specify medications and/or other interventions based on a glucose measurement. The first workflow block may generate a protocol output 570 of the glucose variable as “high glucose” where the variable “glucose measurement” is assigned a value of “high glucose,” “normal glucose,” or “low glucose” depending on the blood chemistry analysis. The second workflow block may then receive as a protocol input 560 the variable “glucose measurement” along with the assigned value of “high glucose.” This value may trigger the second workflow block and define operations within the workflow block. In the absence of inputs and outputs, the first workflow block would provide the variable value to the second user 21, for example, a as a screen displayed value. The second workflow block would then need to provide a prompt to the second user 21 to enter the value. This would increase the interaction between the second user 21 and the clinical guidance UI 250, potentially distract the second user 21 from the task of patient care, and slow down care provision and guidance. Thus, the protocol input 560 and protocol output 570 provide at least the functions of automating transitions between protocols/workflow blocks in order to reduce caregiver interaction and accelerate the provision of critical care and guidance.

The attribute editing window 550 may provide a name field 562 for the input, a format menu 564, and a default value field 566. The default value provided in the default value field 566 enables the clinical guidance engine 235 to mitigate errors if an input or other required value for a protocol or protocol block is not specified or otherwise available when the clinical guidance engine 235 implements the protocol. In an implementation, the clinical guidance engine 235 may recognize that the default value is in use and, in response, prompt the second user 21 to enter an actual, or non-default, value at the UI 250 and/or the clinical guidance engine 235 may generate an error message to alert the second user 21 that only a default value is in use. As another example, some protocols may function properly with a default value and only require a non-default value under special circumstances. For example, a default value of 2 inches (5 cm) for compression depth would apply to all but pediatric patients. The protocol may provide a UI control that would prompt the second user 21 to enter a modifier as needed for the default value. For example, the UI control may ask if or indicate that a patient is pediatric and prompt for a user provided modifier for the compression depth.

The format menu 564 may expand to a drop-down menu 568 to enable the user of the CGPG GUI 220 to specify a numeric, text, or binary input. A numeric input has a numerical value that may be an integer or a non-integer and may have a positive or negative value. A text input is a letter, string of letters, word, combination of words, etc. A binary input may be logically true or logically false (e.g., a value of “true” or “false,” “1” or “0,” “yes” or “no,” “on” or “off,” etc. as examples only and not limiting of the disclosure). For instance, a physiological measurement represented by a variable may be best represented by one of these formats. For example, the variable “pulse rate” may be specified as numeric if the actual value is desired (e.g., for a measured pulse rate of 110 bpm, the numeric value of “pulse rate” would be “110”). Alternatively, the variable “pulse rate” may be specified as text if merely a relative value is desired (e.g., for a measured pulse rate of 110 bpm, the text value of “pulse rate” may be “high”). As another example, the variable “high pulse rate” may be specified as binary (e.g., for a measured pulse rate of 110 bpm, the binary value of “high pulse rate” could be “true” or “1”).

One or more of the input, output, and variable fields may include an add control 569 so that the user may provide a plurality of inputs, outputs, and/or variables. The output menu may include a name field and a format menu. The output menu may not include a default value because the protocol generates the output.

In general, the clinical guidance engine 235 may assign a default value of null to any undefined variable. If for some reason the workflow step that is used to set the value for that variable is not defined or is defined in a workflow branch that is not executed in a particular instance, then the variable would not have a value. If that variable is used in a subsequent workflow step, which would cause an error. Therefore, specifying a default value when defining a variable allows that variable to always have a nominal value. Depending on if the variable is numeric, text, or binary, the default may be a desired nominal condition (e.g., for example, “0” (for numeric), “normal” (for text), or “true” (for binary)).

The variable name 572 is a name applied to a value used within a protocol. The name is only used within a protocol and is not transferred between protocols. For example, a variable name used in a nested workflow block within an envelope protocol is not passed between nested workflow blocks. The variable name 572 may communicate a property of the value. Similarly, to the protocol input 560, the attribute editing window 550 may provide a name field 574 for the variable, a format menu 576, and a default value field 578. Throughout the protocol, the values can be referenced by using the variable name.

In some examples, during execution of a protocol block, the clinical guidance engine 235 may assign a binary value to a variable based on satisfaction of a condition. For example, the clinical guidance engine may receive a measurement of SpO2. The encoded clinical guidance protocol may define a variable “low SpO2” that receives a logically true value based not only on the measurement but also on one or more additional criteria such as a patient's age, gender, and the existence of asthma and/or chronic obstructive pulmonary disease (COPD). As another example, the encoded clinical guidance protocol may define a variable with a text value. The text value may indicate a relative state of the variable (e.g., “high,” “low,” “increasing,” “decreasing,” “improving,” “deteriorating,” etc. as examples only and not limiting of the disclosure). For instance, the variable “SpO2” may receive a value of “high” for a particular combination of measured SpO2, age, and history of asthma and a value of “low” for another combination of these factors. The text value for a relative state may depend on a particular medical context. The variables defined within specific workflows enable the variable values to change dynamically to reflect the particular medical context of a workflow. The workflow may follow a sequence based on the relative value of the variable. For example, an SpO2 value of 90 may be considered low for the general population but normal for a particular patient with a history of asthma. However, the value may be low for that same patient if the measurement occurs immediately after treatment with a nebulizer.

In an implementation, the clinical guidance engine 235 may retrieve information from the resource manager to assign the variable value. For example, variables based on demographics (e.g., gender, age, height, weight) or other information available from clinician input to the clinical guidance UI 250 or a patient charting tool (e.g., the tool 455) may be stored at and retrieved from a resource manager as a variable value. As examples not limiting of the disclosure, the variable “gender” may have a value of “female,” the variable “age” may have a numeric value of “4” or a text value of “pediatric,” the variable “pediatric” for a patient of age 4 may have a binary value of “true,” etc.

In the example above, the clinical guidance engine 235 may use information from the resource manager to determine if a particular SpO2 measurement is high, low, or normal for a patient with a particular age, medical history, skin color, gender, etc. In this manner variables may enable the protocol to tailor itself to the specific patient context. For example, the workflow may define a first specific sequence of actions if the SpO2 measurement is high and a second specific sequence of actions if the SpO2 measurement is low. The sequence of actions may include medications with particular dosages and timing and/or other interventions or measurements. However, the factors that result in the measurement being high, low, normal, or some other relative value can vary from patient to patient and/or between medical circumstances. Variables may enable a hierarchy of determinations so that an evaluation of a particular parameter can change dynamically. For example, a determination that SpO2 is low, high, or normal may be based on a simple look-up table providing an average population value. At a more granular level, the determination may be for a particular patient based on demographics. At a further level of granularity, the determination may account for the condition being treated in the protocol and/or the position of the determination within the protocol (i.e., subject to the current status of medications and/or other interventions).

For example, a value for SpO2 may be a low value based on context. For instance, for a person being treated for sleep apnea with a target of 95%-100%, an SpO2 reading of 92% may be a low value that may trigger further monitoring or analysis. Thus, a protocol for sleep apnea may define “low SpO2” as “true” for a value of 92%. In contrast, an acute respiratory distress (ARF) protocol may be triggered due to an SpO2 value of 88% in a pneumonia patient. At the outset of the ARF protocol, “low SpO2” would have a value of “true” at the outset of the protocol. The ARF protocol may provide guidance and interventions designed to raise the SpO2 to 92% or above for this patient and thus designate “low SpO2” to be “false” at 92% and, for example, enable an exit from an ARF protocol and a return to a routine monitoring protocol for pneumonia. Thus, a particular value, range, or threshold may account for the particular treatment steps performed prior to that threshold being triggered to evaluate whether or not that value, range, or threshold is low or high, normal or abnormal, etc.

Variables are only available in the protocol or workflow block that defines the variable. The indication of whether or not a physiologic indicator or a demographic indicator satisfies the definition of a variable is not saved by the clinical guidance engine 235 in a manner that enables passing this indication between protocols or workflow blocks. In this sense, the variable is a context dependent parameter. An evaluation of a variable may be satisfied only within the context of a protocol. A protocol or workflow block may be directed at a particular treatment and/or patient condition. Thus, for example, what is considered low blood pressure for one particular treatment and/or patient condition may not be the same as for another particular treatment and/or patient condition.

As another example, the name of a variable 572 might be “low systolic blood pressure.” The protocol may apply the default value (or another value provided at a customization GUI 320) and then evaluate the variable according to the applied value. The variable may function as a conditional operator for a protocol block. For example, a condition evaluation block within the protocol using the variable “low systolic blood pressure” may read “if ‘low systolic blood pressure’” and select the output port based on whether or not the systolic blood pressure meets the condition of the variable as defined by the value. To continue the example, the value assigned to “low systolic blood pressure” may be 60-90 mmHg. In this case if the systolic blood pressure is in this range when evaluated at the condition evaluation block by the clinical guidance engine 235, then the clinical guidance engine may proceed according to the sequencing link connected to a condition satisfied output port (e.g., a “yes” port, a “true” port, etc.) of the condition evaluation block. Similarly, if the systolic blood pressure is outside of this range, the clinical guidance engine may proceed according to the sequencing link connected to a condition dissatisfied output port (e.g., a “no” port, a “false” port, etc.) of the condition evaluation block.

As other examples, a variable may indicate that a physiological indicator is improving, deteriorating, or constant (e.g., improving blood pressure, deteriorating heart rate, constant EtCO2, etc.). In some cases, the variable may refer to an indicator that is a category.

This type of variable may be binary, such as, age, gender, pregnant, etc. Such variables are not evaluated relatively (e.g., are not high or low, improving or deteriorating, etc.). Rather these variables are either satisfied or not. For example, a variable “senior” may indicate “yes” if the patient is over 65 and “no” if the patient is under 65. These category variables may determine what procedures are provided to the patient.

Referring to FIG. 5E, examples of examples of assigning clinical guidance protocol attributes for a nested workflow are shown. A quantity of each component in FIG. 5E is an example only and other quantities of each, or any, component could be used. As illustrated in FIG. 5E, a workflow block, for example the bi-level ventilation workflow block 580, may be selected from the protocol block menu 502 and the CGPG GUI 220 may display the selected workflow block in the graphic protocol display area 504. A user selection 581 of the workflow block 580 displayed in the area 504 may cause the CGPG GUI 220 to display the attribute editing menu 582 that is specific to the selected workflow block. For purposes of nesting the workflow block 580 within an envelope protocol, the protocol may include a workflow performance operation (e.g., a “do” operation, an “execute” operation, a “perform” operation, etc. as examples only and not limiting of the disclosure) that directs the clinical guidance engine 235 to branch to a nested workflow if an initiation block conditional evaluation is satisfied. The envelope protocol would invoke the workflow 580 and pass input parameters 561 to the workflow block and receive output parameters 571 at the completion of the workflow block. The specific input parameters 561 and output parameters 571 in the example shown in FIG. 5E were previously specified at the creation of the workflow block 580 using a menu like that shown in FIG. 5D. For example, a user creating the workflow block 580 may specify a minimum target, a maximum target, a temperature evaluation score (e.g., “tempscore”), an alert-verbal-pain-unresponsive (AVPU) evaluation score (e.g., “AVPUscore”), a heart rate score (e.g., “HRScore”) and a respiratory rate score (e.g., “RRScore”) in the input parameter fields 562 in FIG. 5D along with default values for each of these parameters. The scores for these values are based on designated ranges, in this example in the context of bi-level ventilation where a first range gives a score of 0, a second range gives a score of 1, a third range gives a score of 2, etc. The user creating the workflow block 580 may specify a workflow output parameter, such as, for example, “Final Score” in the output parameter field 574 in FIG. 5D. The workflow output parameter may represent the result of the bi-level ventilation that determines the actions taken by a subsequent workflow block that is triggered by or nested within the bi-level ventilation workflow block. At the screen shown in FIG. 5E, a creator of the envelope protocol in which the workflow 580 is nested may change the default values for the input parameters.

The editing menu 582 includes the comment field 852. Comment field 852 captures a user input comment that will appear at the clinical guidance UI 250 in conjunction with other attributes of the workflow block 580. In general, the CGPG engine 210 may include the comment field 852 with any or all workflows or operators in order to enable the clinical guidance UI 250 to provide a customized comment.

Referring to FIG. 5F, examples of parameters used to generate an encoded clinical guidance protocols are shown. A quantity of each component in FIG. 5F is an example only and other quantities of each, or any, component could be used. Unlike the variables described with regard to FIG. 5D, parameters are global in nature and are available to all protocol blocks and workflows within an envelope protocol and between envelope protocols as well. As shown in FIG. 5F, the user specification window 506 for a protocol block may include a parameter menu 585 that enables the CGPG engine 210 to incorporate parameters into the specified protocol blocks. The parameters listed in FIG. 5F are examples only and not limiting of the disclosure. The CGPG engine 210 may provide pre-defined parameters and/or the use of the CGPG GUI 220 may create parameters. The pre-defined and/or created parameters may be stored in a parameter file (e.g., the parameter file shown, for example, in FIG. 13 and described below) that defines attributes of the parameters. In an implementation, the user of the CGPG GUI 220 may edit pre-defined parameters and attributes in the parameter file.

In some examples, parameters are used within various protocol blocks to read physiological status of the patient or the status of a medical device. For example, a protocol block may specify a comparison between two particular parameters and/or between a value of a parameter and a threshold value. Examples of patient status parameters are EtCO2, heart rate, blood pressure, SpO2, etc. An example of a device status is a parameter that can be read to see if physiological closed loop control (PCLC) is enabled. In some implementations, parameters are used within a protocol block to control the functions of a medical device and hence the therapy to the patient. For example, a protocol block may set FiO2 (fraction of inspired oxygen) or turn on PCLC. Some parameters are read-only (i.e., obtain the value from a medical device or caregiver) and some parameters are read-write (e.g., set the value of a parameter at medical device and obtain the value of the parameter from the medical device).

The user of the CGPG GUI 220 may select parameters from the menu 585. For example, the first user 20 may drag and drop a parameter from the menu 585 into the user specification window 506. For example, the drag-drop operation 589 would insert the parameter “low alarm threshold” into the user specification field 588.

When a parameter is used in any of the protocol blocks, the protocol block causes the clinical guidance engine 235 to automatically acquire the parameter from the medical devices communicatively coupled to the clinical guidance engine 235 and in use with the patient. In an implementation, if no medical device is attached to the patient that can measure the parameter and/or the appropriate medical device is not communicatively coupled to the clinical guidance engine 235, then the encoded clinical guidance protocol 225 may cause the clinical guidance engine 235 to provide caregiver guidance at the clinical guidance UI 250. For example, the caregiver guidance may instruct the caregiver to attach a relevant device to the patient and procure a measurement. The caregiver guidance may also instruct the caregiver to communicatively couple the medical device to the clinical guidance engine 235 and/or provide a user entry field so that the caregiver may manually provide the parameter value. For some parameters, it may be possible to measure them manually without a special device (e.g., measuring heart rate using wristwatch). For such parameters, the generated protocol 225 may cause the clinical guidance UI to enable user entry into a textbox.

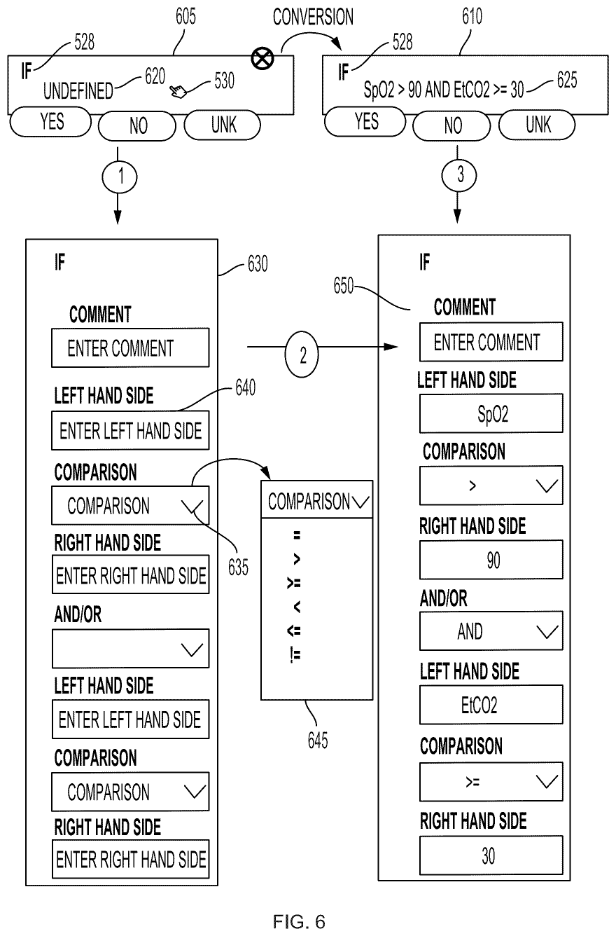

Referring to FIG. 6, an example of a foundation protocol block conversion from unspecified to specified is shown. A quantity of each component in FIG. 6 is an example only and other quantities of each, or any, component could be used. The foundation protocol block 518 is an unspecified protocol block 605 when the user selects the protocol block from the menu 502. Each protocol block includes the operator 528 and an operation. The unspecified protocol block 605 includes an undefined operation 620. A selection 530 of the unspecified protocol block 605 in the graphic protocol display area 504 causes the user specification window 506 to display user specification fields 630 according to a format that corresponds to the operator 528 of the protocol block 605. The user specification fields 630 includes one or more fillable fields. Each fillable field may specify a particular type of entry. For example, the fillable field 640 specifies “left hand side” as the particular type of entry. A field may include a drop-down menu control 635. Selection of the control 635 causes the CGPG GUI 220 to display a drop-down menu 645. The drop-down menu 645 provides selectable entries for the corresponding field. For example, the drop-down menu 645 provides selectable comparators (e.g., >, <, =, etc.) for the condition evaluation operator. Entries to the user specification fields 630 (e.g., the entries shown in the user specification fields 650) cause the CGPG engine 210 to convert the unspecified protocol block 605 to a specified protocol block 610. The specified protocol block 610 includes a defined operation 625.

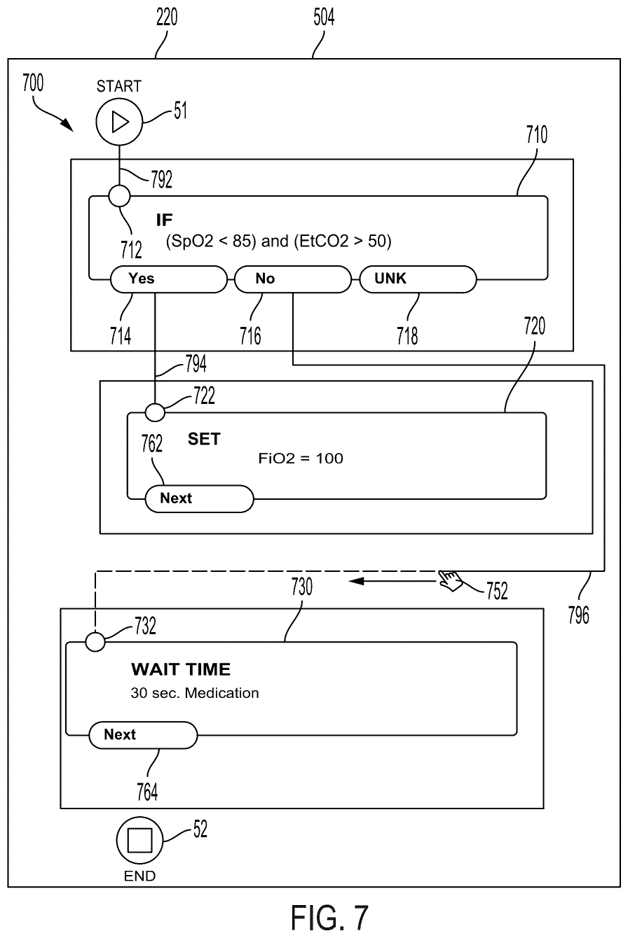

Referring to FIG. 7, examples of input and output ports and sequencing links as used during protocol generation are shown. A quantity of each component in FIG. 7 is an example only and other quantities of each, or any, component could be used. Each foundation protocol block is associated with an input port and one or more output ports.

The protocol 700 includes the start block 51 and the stop block 52. The start block 51 and stop block 52 demarcate the start and end, respectively, of a protocol. If a protocol is automatically triggered by the clinical guidance engine 235 or manually selected at the clinical guidance UI 250 for implementation by the clinical guidance engine 235, the start block 51 and the stop block 52 tell the clinical guidance engine 235 where to start the invoked protocol and when to end the invoked protocol. Without the start block 51, the clinical guidance engine 235 would be unable to determine the initial block to execute to start a protocol. This can be particularly true where a protocol may be a set of looped steps and without a start block 51, the initial step of the loop would be indeterminate. Without a stop block 52, the invoked protocol would remain active in the clinical guidance engine 235. This may cause issues if downstream workflow blocks rely on a block ending or the block is generating values for variables or parameters that are no longer relevant at a downstream point in an envelope protocol. The active protocol block may also continue to generate unnecessary controls or other outputs at the clinical guidance UI 250 and/or continue to generate medical device instructions that are no longer relevant or, worse, incorrect and running counter to a downstream invocation of that medical device.

The protocol under construction includes three foundation blocks, 710, 720, and 730. The protocol block 710 includes the input port 712 and three output ports, 714, 716, and 718. In general, the output ports enable navigation to various branches of the generated protocol. The input port is an entry linkage port that allows a user of the CGPG GUI 220 to connect, or link, the protocol block with one or more preceding blocks by indicating a sequencing link at the CGPG GUI 220. As shown in FIG. 7, the user of the CGPG GUI 220 has indicated a sequencing link 792 from the input port 712 to the preceding start block 51. The one or more output ports are output linkage ports that allow the user of the CGPG GUI 220 to connect, or link, the protocol block with one or more subsequent blocks by indicating a sequencing link at the CGPG GUI 220. As shown in FIG. 7, the user of the CGPG GUI 220 has indicated a sequencing link 794 from the output port 714 of block 710 to the input port 722 of block 720.

The CGPG GUI 220 is configured to display each sequencing link as a line connecting at least two protocol blocks (e.g., a first protocol block and a second protocol block). The sequencing link 796 from output port 716 of block 710 to input port 732 of block 730 is illustrated as being in the process of being indicated by the user. The user icon 752 schematically illustrates the process of indicating the sequencing link 796. At the stage of protocol construction captured in FIG. 7, the output ports 718. 762 and 764 are not linked to a subsequent protocol block. To complete the protocol generation, at least one output port for each protocol block must be sequenced to at least one input port. Additionally, the stop block 52 must be linked to one or more final protocol blocks. Thus, in the example of FIG. 7, the ports 762 and 763 will be linked to protocol blocks added to the graphic protocol display area 504 and/or to the stop block 52. The stop block 52 will be linked to one or more final protocol blocks to complete protocol generation. The output port 718 may optionally be linked to a subsequent block or may be set to a null value to deactivate the output port.

For a data interchange format file, the CGPG engine 210 may encode a protocol block as step objects, such as, for example:

“type”: “if”,
“lhs”: “SpO2”,
“comparison”: “>”,
“rhs”: 85
“transitions”:
{“true”: “node-1”,
“false”: “node-2”,
“unknown”: “node-3”}

This example would identify a type of foundation block operator (e.g., “if”) and then define the left- and right-hand sides of a comparison (e.g., a greater than comparison). If the comparison is true then a first output port is invoked (e.g., “node-1” or port 714), if the comparison is false then a second output port is invoked (e.g., “node-2” or port 716), and if the comparison if unknown then a third output port is invoked (e.g., “node-3” or port 718). In the example of a data interchange format file, the CGPG engine 210 may assign a unique identifier to every input node for every protocol block. For example, block 710 may correspond to a first unique identifier, UI1, block 720 may be correspond to a second unique identifier, UI2, block 730 may correspond to a third unique identifier, UI3, etc. When the sequencing links are provided graphically at the CGPG GUI 220, the CGPG engine 210 assigns the unique identifier of the input port to the preceding and connected output port. For example, the CGPG engine 210 would assign UI2 for block 720 to port 714 (e.g., node-1-UI2) and would assign UI3 for block 730 to port 716 (e.g., node-2=UI3), etc. Any time the user of the CGPG GUI 220 changes a sequencing link, the CGPG engine 210 replicates that change by changing the unique identifier assignments. Thus, the sequencing links are not merely visual connectors but rather represent encoding the sequencing instructions to generate the encoded clinical guidance protocol 225.

The types of output ports for a particular protocol block are determined by the operator. In general, each of the one or more output linkage ports is associated with a sequencing attribute. Examples of sequencing attributes include yes, no, next, guidance, unknown, etc. as discussed in further detail with regard to FIGS. 8A-1-8H. The sequencing attribute indicates a sequencing relationship between the linked protocol blocks. The CGPG engine 210 does not require a user of the CGPG GUI 220 to link every output linkage port of a protocol block to an input port. During execution, the clinical guidance engine 235 ignores the attribute of an output port that is not linked to an input port. For example, as described below, a condition evaluation protocol block may include an indeterminate value port (e.g., an “unknown” port, an “value unavailable” port, an “unspecified” port, etc. as examples only and not limiting of the disclosure) to provide a sequencing path if the condition evaluated by the block is indeterminate. However, if the indeterminate value port is not linked to any other port, then the protocol has likely ensured that a value is available (e.g., by specifically setting a value that is provided to the condition evaluation block) and the indeterminate value port is moot and causes no action by the clinical guidance engine 235.

Referring to FIGS. 8A-1-8E-3, examples of foundation protocol blocks and corresponding user specification windows are shown. A quantity of each component in FIGS. 8A-1-8E-3 are examples only and other quantities of each, or any, component could be used. These foundation blocks are examples only and not limiting of the disclosure. In an implementation, the CGPG GUI 220 may provide additional foundation blocks other than those exemplified herein and/or may provide a subset of the foundation blocks exemplified herein. Each foundation protocol block shown in FIGS. 8A-1-8E-3 is associated with an operator. The types of entries to the user specification window 506 are determined by the operator.

Referring to FIG. 8A-1, examples of a range check operator 870 and a match evaluation operator 91 are shown. Foundation protocol block 805a shows an example of a range evaluation operator 870. The specification window 805b for the range evaluation operator 870 includes the fields comment, parameter/variable name, and a lower level and an upper level for each of one or more ranges. The specification window 805b also provides a selectable add control 806 to increase the number of range choices and a selectable remove control 807 to decrease the number of range choices. The range evaluation operator causes the clinical guidance engine 235 to evaluate a range for one or more continuous parameters or continuous variables and select an output port that leads to a subsequent protocol block as defined by the sequencing link connecting the output port and the input port of the subsequent protocol block. A continuous parameter or continuous variable may have a range of possible values that are discrete only to a particular number of significant figures. For example, normal body temperature may range between 36 degrees Celsius and 38 degrees Celsius with discrete values within that range being determined by the accuracy of the thermometer. For instance, the discrete values may be tenths of degrees, hundredths or degrees, etc. The output ports for the range evaluation operator are a first output port 808a if the parameter or variable is in a first range, a second output port 808b if the parameter or variable is in second range, and an indeterminate value port 809. The indeterminate value port 809 provides an action if the range cannot be determined, for example, due to a lack of appropriate data. For example, the condition evaluated by a protocol block may require a value of a particular physiological parameter. However, if a medical device configured to measure the particular physiological parameter is not available or is not communicatively coupled to the clinical guidance engine, then the protocol block may default to the indeterminate value port and follow a sequencing path to a protocol block configured to acquire the particular physiological parameter from a caregiver (e.g., a UI instruction protocol block).

Foundation protocol block 92a shows an example of a match evaluation operator 91. Unlike the range check operator 870, the match evaluation operator 91 evaluates discrete numbers or text strings. An assessment score like MEWS or qSOFA provide examples of discrete numbers. A text string may be a variable or parameter defined in a protocol block. For example, the text string may be an input parameter 560, and output parameter 570, or a variable 574 as shown in FIG. 5D. The specification window 92b for the match evaluation operator 90 includes the fields comment, parameter/variable name, a first match condition 98, and a second match condition 99. The specification window 92b also provides a selectable add control 95 to increase the number of match conditions and a selectable remove control 96 to decrease the number of match conditions. As an example, the editing window 550 in FIG. 5D may define a parameter of “vent mode” for the operation mode of a ventilator. The values for “vent mode” may be “CPAP,” “BiPAP,” “volume control,”, and “pressure control.” In an implementation, the match value operator 91 may evaluate if the “vent mode” exactly matches “CPAP” as match A or “BiPAP” as match B. If match A is satisfied, then the protocol proceeds from output port 93a. If match B is satisfied, then the protocol proceeds from output port B. If neither is satisfied, then the protocol proceeds from output port 94. As another example, the editing window 550 may define an alert or instruction variable and when the alert or instruction variable matches a particular value (e.g., match A or match B), the protocol proceeds according to the respective output port. For instance, if the variable defined as “alertText” for a user alert is equal to “Low SpO2 with tachycardia” the protocol may proceed to port 93a for instructions or other protocol guidance for this alerted condition. A match with “Low SpO2 with bradycardia” may proceed to port 93b for instructions relevant to this alerted condition.

Referring to FIG. 8A-2, examples of a condition evaluation operator 528 and a value assignment operator 871 are shown. Foundation protocol block 810a shows an example of the condition evaluation operator 528. The specification window 810b for the condition evaluation operator includes the fields comment, left hand side, comparison, right hand side, and/or, left hand side, comparison, and right-hand side. The condition evaluation operator causes the clinical guidance engine 235 to evaluate one or more conditions that compare the left- and right-hand sides of an expression according to the selected comparator. The left- and right-hand sides may include literal values (e.g., a numeric value or a binary value), parameter names, variable names, or expressions. Expressions are discussed below in regard to FIGS. 8B-1 and 8B-2. Additionally, the evaluation is subject to an and/or logic selection. The evaluation causes the clinical guidance engine 235 to select an output port that leads to a subsequent protocol block as defined by the sequencing link connecting the output port and the input port of the subsequent protocol block. The output ports for the condition evaluation operator are a first output port 812a if the one or more conditions are satisfied, a second output port 812b if the one or more conditions are not satisfied, and the indeterminate value port 809. The indeterminate value port 809 provides an action if the conditions cannot be evaluated, for example, due to a lack of appropriate data. In this manner, the condition evaluation operator 528 creates branches in the protocol based on the evaluated condition.

Foundation protocol block 815a shows an example of a value assignment operator 871. The specification window 815b for the value assignment operator includes the fields comment, parameter/variable name, and a value for the parameter/variable. The specification window 815b also provides a selectable add control 816 to increase the number of set choices and a remove control 817 to decrease the number of set choices. The value assignment operator causes the clinical guidance engine 235 to perform an internal protocol operation. Specifically, this operator functions to set the parameter or variable to a particular value. The output port for the value assignment operator is an advance port 818 (e.g., a “next” port, a “continue” port, an “advance” port, a “proceed” port, etc. as examples only and not limiting of the disclosure) that leads to a subsequent protocol block as defined by the sequencing link connecting the output port and the input port of the subsequent protocol block. When the value assignment operator is applied to a parameter in use by a medical device or a caregiver, the treatment provided to the patient according to that parameter changes in response to the value assignment operation. For example, the value assignment operator may define an adjustment amount for an inspiratory positive airway pressure (IPAP) value for a ventilation device. The protocol that includes the value assignment operation may cause the clinical guidance engine 235 to adjust IPAP by a particular amount (e.g., “set IPAP to IPAP +2”). As another example, based on a blood pressure measurement, a MEWS protocol may set a value of the blood pressure score variable to a particular numeric value.

Referring to FIGS. 8B-1 and 8B-2, examples of a timer operator 872, a computation operator 874, and a delay operator 873 are shown. As shown, for example in FIG. 8B-1, foundation protocol block 820a shows an example of a timer operator 872. The specification window 820b for the timer operator includes the fields comment and a duration. The timer operator causes the clinical guidance engine 235 to arm a timer for the specified duration. The output ports for the timer operator include the advance port 818 that leads to a subsequent protocol block following the duration of the timer and as defined by the sequencing link connecting the output port and the input port of the subsequent protocol block. The output ports for the timer operation include a timer expiration port 819 that signals the expiration of the timer. A sequencing link from the timer expiration port determines protocol steps that the clinical guidance engine 235 may implement at the expiration of the timer.

As further shown, for example, in FIG. 8B-1, foundation protocol block 830a shows an example of a computation operator 874. The specification window 830b for the computation operator includes the fields comment and expression. The computation operator causes the clinical guidance engine 235 to perform an internal protocol operation. Specifically, this operator functions to compute a mathematical equation. For example, the protocol may define a risk score to be the sum of parameters A, B, C, and D. A computation operator 874 in this example may add parameters A, B, C, and D and pass this result to a value assignment block that sets the value of risk score variable to the sum. The output port for the computation operator is the advance port 818 that leads to a subsequent protocol block as defined by the sequencing link connecting the output port and the input port of the subsequent protocol block.

As shown, for example, in FIG. 8B-2, foundation protocol block 821a shows an example of a delay operator 873. The specification window 821b for the delay operator includes the fields comment, display text 940, a duration 941, and a conditions control 942. The delay operator causes the clinical guidance engine 235 to provide the display text at the clinical guidance UI 250 for the duration of the specified wait time. The output port for the delay operator is the advance port 818 that leads to a subsequent protocol block following the duration of the wait time and as defined by the sequencing link connecting the output port and the input port of the subsequent protocol block. In an implementation, the user 20 of the CGPG GUI 220 may invoke the conditions control 942 to add conditions to the delay operator. Selection of the conditions control 942 may cause the CGPG engine 210 to provide the specification window 821c at the GUI 220. The window 821c further includes conditional definition field 943 for a selection of an “and” Boolean operator or an “or” Boolean operator to allow for the delay time to operate with or as an alternative to a satisfied condition. The fields 944, 945, and 946 define the left side, the comparative operator, and the right side of the condition, respectively. For example, a protocol may provide an instruction operator for albuterol delivery followed by a delay operator. The delay operator may provide instructions based on added conditions to wait five minutes before the next dose or if the SpO2 level is greater than 92, to stop waiting and skip the next dose. As another example, the conditions may evaluate trends of SpO2 and provide instructions other than waiting for five minutes if the SpO2 is trending down. Thus, the conditions enable various customized pathways for implementing and exiting a delay in further treatment.

A delay operation differs from a timer in that a delay operation adds a delay period during which the caregiver may not receive any task or intervention instructions. In contrast, a timer operator tracks a duration of time until a particular next step may be performed but the caregiver may receive other instructions for tasks or interventions during that duration of time. The sequencing link that extends from the advance port 818 defines the operations that occur after the timer is set but that may occur in parallel with the timer countdown. Thus, the thread invoked by the sequencing link from the advance port 818 runs in parallel with the timer. At the expiration of the timer, the caregiver and/or a medical device may receive other instructions. For example, a caregiver or medical device may provide an intervention and then the protocol may start a timer. During the timer countdown, the caregiver and/or medical device may perform other tasks but at the expiration of the timer, the protocol may cause the clinical guidance engine to check some value to determine the efficacy of the intervention. This may be helpful, for example, for interventions where the body needs some time to respond in order to determine if the intervention was successful. For example, the clinical guidance engine 235 may need to determine an effect of an increase in ventilation rate or volume may need to be verified by checking EtCO2 values, but not until after a couple of minutes have transpired after the increase because of a delay in physiological response to the increase. Thus, the verification step may link to the timer expiration port 819 while a temperature measurement may link to the advance port 818 where the temperature measurement can be performed while waiting to determine the effect of the ventilation increase. The advance and timer expiration ports open two parallel threads based on sequencing links connected to these ports. The advance port causes a thread to run immediately after the timer is armed and the timer expiration port causes a thread to run at the end of the time specified for the timer.

Referring to FIG. 8C, examples of an alert operator 875 and a check list operator 876 are shown. Foundation protocol block 835a shows an example of an alert operator 875. The specification window 835b for the alert operator includes the fields comment, text, and a timeout duration. The alert operator causes the clinical guidance engine 235 to provide the text at the clinical guidance UI 250 as a caregiver alert for the timeout duration. The output port for the alert operator is the advance port 818 that leads to a subsequent protocol block following the duration of the timeout and as defined by the sequencing link connecting the output port and the input port of the subsequent protocol block.

Foundation protocol block 840a shows an example of a checklist operator 876. The specification window 840b for the checklist operator includes the fields comment, title, and one or more option fields each associated with a variable. The specification window 840b also provides a selectable add control 841 to increase the number of check list options and a remove control 842 to decrease the number of check list options. The specification window 840b provides multiple variables and the value of the variable is set to a logically true value when the caregiver indicates at the clinical guidance UI 250 that the check list item is complete. If no indication is made at the clinical guidance UI 250, then the value of the variable is set to a logically false value. The checklist operator causes the clinical guidance engine 235 to display specified items at the clinical guidance UI 250 and to assign a variable a logically true value if a specified item is marked as complete by the caregiver at the clinical guidance UI 250 or assign a logically false value if the specified item is not marked as complete. The output port for the checklist operator is the advance port 818 that leads to a subsequent protocol block following the duration of the timeout and as defined by the sequencing link connecting the output port and the input port of the subsequent protocol block.

Referring to FIG. 8D, examples of a multiple-choice operator 877 and a reference operator 882 are shown. Foundation protocol block 845a shows an example of a multiple-choice operator 877. The specification window 845b for the multiple-choice operator includes the fields comment, title, and one or more options. The specification window 845b also provides a selectable add control 846a to increase the number of check list options and a remove control 846b to decrease the number of multiple-choice options. The options are each a caregiver task available for selection. The multiple-choice operator causes the clinical guidance engine 235 to provide the one or more options at the clinical guidance UI 250 and select an output port that leads to a subsequent protocol block as defined by the sequencing link connecting the output port and the input port of the subsequent protocol block. The output ports for the multiple-choice operator are a first output port 848a for a first option selection, a second output port 848b for a second option selection, and one or more additional output ports for optional additional option selections. Each output port leads to a subsequent protocol block as defined by the sequencing link connecting the output port and the input port of the subsequent protocol block.

Foundation protocol block 851a shows an example of a reference operator 882. The specification window 851b for the reference operator includes the fields comment 852, reference 853, and browse 85. In an example, a guidance operator (e.g., the guidance operator shown in FIG. 8F-2) may incorporate the reference operator 882. The reference operator 882 The reference operator causes the clinical guidance engine 235 to access and provide material from the instruction libraries 322, 324, and/or 326 in the resource manager 240, 260, and/or 280. The clinical guidance engine 235 may provide the material (e.g., display and/or provide audio) at the at the clinical guidance UI 250. The comment field 852 captures a user input comment that will appear at the clinical guidance UI 250 in conjunction with the reference materials. The reference field 853 captures a link to the reference material as entered by the user. The browse field 85 enables a user to browse files in the resource manager 240, 260, and/or 280 and select a link. The reference field 853 displays the selected link. The output port for the reference operator is the advance operator 818. The output port leads to a subsequent protocol block as defined by the sequencing link connecting the output port and the input port of the subsequent protocol block.

In an implementation, the additional guidance available through a reference operator 882 may be provided by accessing and displaying all or a portion of one or more documents, pictures, videos, or other reference files with instructions or information relevant to a protocol or protocol step. For example, the reference file may include a set of instructions and/or clinical guidance protocols in a document image. The reference file may be in various formats, including but not limited to, portable document format (PDF), a binary file format (e.g., a DOC file or other word processing format), a spreadsheet format (e.g., a XLS or other spreadsheet file format), joint photographic experts group format (JPEG) or other image format, an audio file format (e.g., MP3, WMA, PCM, WAV, AIFF, etc.), and/or a video file format (e.g., MP4, MOV, AVI, WMV, etc.), In various implementations, the additional instructions may include material from the instruction libraries 322, 324, and/or 326 in the resource manager 240, 260, and/or 280.

Referring to FIG. 8E-1, an example of a trend operator 878 is shown. Foundation protocol block 850a shows an example of the trend operator 878. The specification window 850b for the trend operator includes the fields comment, input parameter, trend type, window size, and output variable. The trend operator causes the clinical guidance engine 235 to perform an internal protocol operation. Specifically, this operator functions to retrieve previously stored values of a particular physiological parameter over a particular time period, calculate differences between those values, and display or summarize those differences as a trend. For example, the trend operator may cause the clinical guidance engine 235 to display a graph of the change in a value over a period of time and/or display a text summary of the trend. For example, the text summary may be “increasing,” “decreasing,” “stable,” “no change,” etc. as examples only and not limiting of the disclosure. The text summary may also indicate a rate of change using a word to describe an increase or decrease. For example, a steep change in a parameter may be represented by a display of “rapidly decreasing,” “rapidly increasing,” “precipitous decrease,” etc. as examples only and not limiting of the disclosure. The output ports for the trend operator are the advance port 818 and the indeterminate value port 809. The advance port 818 leads to a subsequent protocol block defined by the sequencing link connecting the output port and the input port of the subsequent protocol block. The indeterminate value port 809 provides an action if a physiological value measured by a medical device 265 is unknown to the clinical guidance engine 235. In such a situation, the protocol may define, via sequencing links to the indeterminate value port 809, operations for the clinical guidance engine 235. These operations may include an automatic prompt at the clinical guidance UI 250 for the second user 21 to set up and activate a sensor, a request at the clinical guidance UI 250 for entry of a value, an instruction to a medical device to provide the value to the clinical guidance engine 235, and/or an instruction along with guidance for coupling a particular medical device to the clinical guidance engine 235.

Referring to FIG. 8E-2, examples of a UI instruction operator 880 and a UI user input operator 881 are shown. Foundation protocol block 860a shows an example of a UI instruction operator 880. The specification window 860b for the UI instruction operator includes the fields comment, title, and one or more instruction bullet points. The specification window 860b also provides a selectable add control 861 to increase the number of bullet point instruction options and a remove control 862 to decrease the number of bullet point instruction options. The UI instruction operator causes clinical guidance engine 235 to provide a list of instructions at the clinical guidance UI 250 according to the user specification entries. Optionally, each instruction is displayed with a bullet point symbol to identify individual instructions. The output ports for the UI instruction operator include the advance port 818 that leads to a subsequent protocol block following the display of the drug administration instruction and as defined by the sequencing link connecting the output port and the input port of the subsequent protocol block. In an example, a guidance operator (e.g., the guidance operator shown in FIG. 8F-1) may incorporate the instruction operator 880. The output ports for the UI instruction operator may also include a guidance port 863 (e.g., a “guidance” port, an “instructions” port, a “help” port, etc. as examples only and not limiting of the disclosure) that enables tiered guidance. The guidance port 863 is described in detail in regard to FIG. 8F-1.

Foundation protocol block 865a shows an example of a UI user input operator 881. The specification window 865b for the UI user input operator includes the fields comment, title, display text, input type, and variable. The UI user input operator causes clinical guidance engine 235 to provide a user entry prompt at the clinical guidance UI 250 according to the user specification entries. The output ports for the UI user input operator include an entry confirmation port 866 that leads to a subsequent protocol block following a user entry at the clinical guidance UI 250 and as defined by the sequencing link connecting the output port and the input port of the subsequent protocol block. The confirmation port 866 generates an entry confirmation control at the clinical guidance UI 250. The user of the clinical guidance UI 250 may provide the user entry and then use the entry confirmation control to confirm the entry. The output ports for the UI user input operator also include an entry cancelation port 867. The entry cancelation port 867 generates an entry cancelation control at the clinical guidance UI 250. The user of the clinical guidance UI 250 may provide the user entry and then use the entry cancelation control to erase and re-enter the user entry. The entry cancelation port 867 also enables the user of the CGPG GUI 220 to specify protocol steps subsequent to the user input block 865a even in the absence of a user entry or in the case of an erroneous entry. Thus, the clinical guidance engine 235 will not stall or generate an error in the guidance in these situations because the subsequent steps as indicated by a sequencing link from the entry cancelation port 867 provides a corrective operation.

Referring to FIG. 8E-3, an example of a medication administration operator 879 is shown. Foundation protocol block 855a shows an example of a medication administration operator 879. The medication administration operator 879 causes the clinical guidance engine 235 to refer to a drug list and check for dosages allowed given demographics in the resource manager 240, 260, and/or 280. Additionally, the protocol block with the medication administration operator defines dosage, number of doses, and time between doses. A protocol may leverage the time between doses to trigger alerts after time between dosages expires. The medication administration operator is a similar to the UI instruction operator 880 described below but is specifically directed at instructions for administration of medication and enables use of the appropriate drug resources in the resource manager 240, 260, and/or 280. The output port for the medication administration operator is the advance port 818 that leads to a subsequent protocol block following the display of the drug administration instruction and as defined by the sequencing link connecting the output port and the input port of the subsequent protocol block. In an implementation, the medication administration operator may include the guidance port 863. The guidance port 863 may cause the clinical guidance engine 235 to provide a guidance control that enables a user of the clinical guidance UI 250 to request dosage and/or administration guidance for a particular medication. In an implementation, the medication administration protocol block 855a may cause the clinical guidance engine 235 to retrieve medication information from the medication library 1230 and provide the medication information at the clinical guidance UI 250

The specification window 855b for the medication administration operator includes the fields drug name 62, dosage 63, number of doses 64, interval between doses 65, maximum number of doses 66, confirmation count variable 67, and cancellation variable 68. Based on each of these user specification entries, and as shown for example in FIG. 8E-4, the medication administration operator causes the clinical guidance engine 235 to provide drug administration instruction and guidance at the clinical guidance UI 250 according to the user specification entries. The clinical guidance UI 250 may provide instructions for each step of a multi-step drug administration procedure.

Referring to FIG. 8E-4, an example of UI features corresponding to a medication administration operator is shown. A quantity of each component in this figure is an example only and other quantities of each, or any, component could be used. In an implementation, the clinical guidance UI 250 includes a drug administration clinical guidance UI control 40. The drug name provided at block 62 appears in block 41 of the control 40. In this example, if the drug name “albuterol sulfate” is entered into field 62, then “albuterol sulfate” appears in block 41. The dosage entered into field 63 appears in block 43 of the control 40. In this example, if the dosage is “1 puff” of an inhaled medication, then “1 puff” appears at the block 43. The number of doses entered into field 64 appears in block 44 of the control 40. In this example, if the number of doses is “5”, then “5” appears at the block 44. The block 44 may also provide a dose count. Thus, each dose is a particular number of the number of doses indicated in block 64. For example, if the number of doses in block 64 is “1” then block 44 will indicate “dose 1 of 1” after the administration of one dose. As another example, if the number of doses in block 64 is “5” then block 44 will indicate “dose 1 of 5” after the administration of one dose. In the example shown in FIG. 8E-4, the block 44 shows “dose 3 of 5” after the administration of three doses for a number of doses equal to five. The interval between doses provided at block 65 appears in block 42 of the control 40. In this example, if the interval of 60 seconds is entered into field 65, then block 42 will show a dose interval timer 42. The dose interval timer 42 may be a countdown timer that starts at 60 seconds and counts down until it is time to administer the next dose. In this example, the interval is provided in seconds, but other time units of seconds, minutes, hours or combinations thereof are within the scope of the disclosure.

The maximum number of doses provided at block 66 defines a parameter. The user of the clinical guidance UI 250 may indicate deliver of a dose with the medication delivery confirmation control 45. The delivery confirmation control 45 may increment a counter variable every time the medication is administered. The field 67 provides the name of the counter variable. The maximum number of doses provided at field 66 may control how many times the drug administration clinical guidance UI control 40 appears at the UI 250 for a particular instance of the medication administration operator 879. For example, if the maximum number of doses is five and the number of doses is five, then the drug administration clinical guidance UI control 40 may appear at the UI 250 only one time for the particular instance of the medication administration operator 879. However, if the maximum number of doses is fifteen and the number of doses is five, the drug administration clinical guidance UI control 40 may appear at the UI 250 three times. Each instance of the drug administration clinical guidance UI control 40 corresponds to a particular medication as named in the drug name field 62.

Referring to FIG. 8E-5, an example of a protocol generation sequence with a medication administration operator is shown. A quantity of each component in this figure is an example only and other quantities of each, or any, component could be used. The block 75 of the protocol generation sequence 70 provides an example of an implementation of the maximum number of doses field 66. In this block, a counter variable “drugCount” as provided by field 67 is incremented with each use of the delivery confirmation control 45 is compared with a parameter defined by the field 66, namely the maximum number of doses shown in this example as the variable “maxdose.” As long as the counter variable is less than the maximum number of doses, the block 75 allows the “yes” port 76 to enable a repeat of the medication administration operator in block 71. However, when the counter variable equals the maximum number of doses, the “no” port 77 ends the sequence 70 and no further instances of the control 40 appear at the UI 250 for the drug named in block 71. In an implementation, the sequence 70 may include an alert operator 875 sequenced with the “no” port 77 in order to generate an alarm when the maximum number of doses is reached. In an implementation, a medical device 265 may control the medication administration and the block 75 may cause the clinical guidance engine 235 to send a signal to the medical device 265 to repeat or discontinue the medication administration.

The control 40 may further include a cancellation control 46. The user of the UI 250 may implement the cancellation control 46 in order to interrupt a medication delivery where the patient is not tolerating the medication, the medication has no effect on the patient, and/or the condition targeted by the medication is deteriorating and not improving. The cancellation control 46 may set a variable with a default of false to a value of true when a user of the interface 250 implements the cancellation control 46. The field 68, shown in FIG. 8E-3, may define the variable controlled by the cancellation control 46. As shown in FIG. 8E-5, the cancellation variable from field 68 may be “drugCancel” as shown in block 72. As long as this variable remains at a default value of false, the protocol sequence 70 allows for the medication administration to continue up to the maximum number of doses. For example, as long as “drugCancel” is not equal to true (i.e., is equal to false), the sequence proceeds from block 72 to block 74. In this example, the block 74 may cause the UI 250 to display an instruction to “monitor the patient for improvement.” However, if the variable “drugCancel” is equal to true, then the sequence proceeds from block 72 to block 73 in order to proceed to a new set of protocol steps through the advance port 818. The new set of protocol steps may be determined by the user of the CGPG GUI 220 as protocol instructions in the event that the user of the UI 250 invokes the cancellation control 46.

Referring again to FIG. 8E-4, the UI 250 may include an instruction scroll control 48 and/or a connected devices window 49. The instruction scroll control 28 may enable a user of the UI 250 to scroll between multiple available instructions.

The connected devices window 49 may identify the medical device(s) 265 coupled to the mobile device 255. In an implementation, the clinical guidance engine 235 may modify or adjust a patient care protocol based on the available equipment. In an implementation, the clinical guidance engine 235 may display information from a medical device at the UI 250 for a medical device communicatively coupled to the mobile device 255. For example, the UI 250 in FIG. 8E-4 shows information that may be displayed at a patient monitor or a patient monitor-defibrillator. The patient monitor or patient monitor-defibrillator (e.g., the patient monitor-defibrillator 482) may provide, for example, one or more of an electrocardiogram (ECG) waveform 30, an EtCO2 waveform 31, and SpO2 waveform 32, a discrete reading for EtCO2 38, a discrete reading for SpO2 37, heart rate 39, body temperature 33, non-invasive blood pressure (NIBP) 36 and/or one or more invasive blood pressure channels 34 and 35 (e.g, for one or more of arterial, venous, or intracranial pressure data).

In an implementation, the mobile device 255 may couple with a first or initial medical device, for example, at the outset of patient care. Subsequently, other devices (e.g., at least one additional medical device) may establish communications with the mobile device 255. The mobile device 255 may identify the subsequent devices based on the communicative coupling and the clinical guidance engine 235 may update the connected devices window 49 to include the identification of these subsequent devices as they are added to the system. In an implementation, the clinical guidance engine 235 may control the UI 250 and display at the mobile device 255 to provide physiologic parameters from the initial medical device at the UI and display. When an additional medical device is coupled to the mobile device 255, the mobile device 255 may receive additional physiologic parameters from that additional device and, in response, may modify the UI 250 in real-time to include one or more of the additional physiologic parameters.

The medical device data displayed in the example in FIG. 8E-4 may be a visual reproduction of that information displayed at a display interface of the medical device(s) 265 when one or more of the following visual elements of the display interface of the medical device(s) 265 is reproduced at the UI 250: the display layout, magnification of each data section, physiologic waveform selection, physiologic numeric readout selection, resolution, waveform duration, waveform size, text size, font, and display colors. In some examples, the visual reproduction of the display screen of the medical device(s) 265 at the mobile device 255 can exactly replicate at is displayed at the medical device(s) 265 or one or more of the visual elements may be altered from what is displayed at the medical device(s) 265. In one example, font and size of one or more items of displayed case information may be enlarged and/or highlighted to draw the eyes of users of the mobile device 255 to the respective case information. Waveforms or other data of a certain type may be magnified or color coded in a particular fashion. For example, ECG waveforms, such as in a 12-lead analysis, may be magnified similarly, or certain waveforms may be emphasized in resolution, color, or other manner of reproduction. Alternatively, in some embodiments, numerical values representing non-continuous physiological measurements (e.g., NIBP, SpO2, HR, and/or EtCO2) may be shown in a similar sized font or layout. In some cases, for example, a visual reproduction may be a replication of the information as presented on the display interface of the medical treatment device, or alternatively with slight variations thereof.

In some examples, the UI 250 may include one or more selectable inputs at the display interface that cause more, different, or additional information to be displayed, cause one or more actions to be taken at the medical device(s) 265, or provide additional user input interface screens that allow users to submit information that can be transmitted to the medical device(s) 265.

In some examples, the visual reproduction may encompass an exact replication of the data displayed at the medical treatment device. In other examples, the visual reproduction may include data and formatting variations that can enhance viewing and comprehension of the case information by the mobile device user. In some examples, display layout, magnification of each data section, physiologic waveform selection, physiologic numeric readout selection, resolution, waveform duration, waveform size, text size, font, and/or display colors may vary from what is displayed at the medical treatment device(s).

In some implementations, the information displayed at the UI screen 250 may vary from the information displayed at a display interface of the medical device(s) 265. In some examples, the differences between the interfaces can include differences in a type of information displayed, a display layout, or a display format. For example, an amount of magnification of each data section, resolution, size, and screen position and/or relative waveform sizes and colors, fonts, and text size may vary between the UI 250 and the display interface of the medical device(s) 265.

Referring to FIG. 8F-1, an example of a guidance operator is shown. A quantity of each component in this figure is an example only and other quantities of each, or any, component could be used. Foundation protocol block 885a shows an example of the guidance operator 886. The guidance operator 886 implements a series of one or more instruction operators 880 and/or one or more reference operators 882. The specification windows 860b and 851b, for the instruction and reference operators respectively, provide the specific attributes of the guidance operator 886. The foundation protocol block 885a includes an entrance port 898a and an output port 898b. When an encoded clinical guidance protocol 225 invokes a guidance operator 886, the entrance port 898a defines a beginning of the series of instruction operator(s) and/or reference operator(s). For example, in the sequence 885b, the guidance operator includes two instances 886a and 886b of the instruction operator and once instance 886c of the reference operator. In this example, instruction block 886a provides a notification that a patient is in shock and then instruction block 886b provides an instruction to administer oxygen to the patient. In practice, the guidance block 885b would be preceded by protocol blocks evaluating physiological parameters to determine if the patient is in shock. The reference block 886c enables the clinical guidance engine 235 to access and provide material relevant to oxygen administration for a patient in shock from the instruction libraries 322, 324, and/or 326 in the resource manager 240, 260, and/or 280. The entry in the reference block provides the link to the particular reference materials relevant to the preceding instructions.

As shown in FIG. 8F-1, the guidance operator may include one or more nested guidance ports (e.g., the guidance ports 887a and 887b) associated with instruction operators. These nested guidance ports correspond to respective guidance operators and attributes. In this manner, a user of the clinical guidance protocol generation GUI 220 may customize the guidance provided in conjunction with a clinical instruction. The nested guidance ports 887a and 887b enable the user of the GUI 220 to customize the level of detail available to the user 21 of the clinical guidance UI 250. The nested guidance operators enable tiered clinical guidance in which the user 21 of the clinical guidance UI 250 can request more or less information and reference materials depending on their level of training and expertise.

Referring to FIG. 8F-2, an example of tiered clinical guidance UI features corresponding to guidance and reference operators in an encoded clinical guidance protocol is shown. A quantity of each component in this figure is an example only and other quantities of each, or any, component could be used. In this example, the instruction blocks 823a, 825a, and 827a provide three layers of guidance. With instruction block 823a, for example, the clinical guidance UI 260 may provide the instruction “ventilate patient with bilevel” 823b as specified in the instruction block 823a. In an implementation, the guidance port 863 in the instruction block causes the clinical guidance engine 235 to provide a guidance request UI control 824 or 826 at the clinical guidance UI 250 that enables the caregiver to request help and guidance. By enabling a generation of a guidance request UI control 824 or 826, the guidance port 863 allows for a workflow to provide guidance details on demand and, therefore, only if a user of the clinical guidance UI 250 desires more details. In various examples, the UI control (e.g., 824 or 826) generated by the guidance port 863 could provide an option to call on an outside resource for help (e.g., a telephone call or text message to a supervisor or physician), access telementoring, and/or provide suggestions for diagnostic steps for the user to perform.

The guidance controls 824 and 826 may be configured to capture a user selection and, in response to the user selection, the encoded clinical guidance protocol may enable the clinical guidance UI 250 to provide more detailed guidance corresponding to the instruction. For example, a first instruction 825b of “turn on ventilator” may be provided to the user based on the instruction block 825a. However, if the user knows how to implement an instruction without further guidance, then the clinical guidance UI 250 may not display any further details.

Instruction block 827a provides an example of a further level of instruction detail available at the UI 250 (e.g., the instruction 827b) in response to a user selection of the guidance control 826. Additionally, reference block 828a provides an example of a reference operator that retrieves a diagram from the file or storage location 829b associated the link 829a. The clinical guidance UI 250 may display the graphic 828b available from the reference link 829a. This reference material provides a further level of detailed guidance for a user, for example, that is unfamiliar with the ventilator controls and requires graphic guidance.

Referring to FIGS. 8G-8H, examples of foundation protocol blocks and corresponding user specification windows are shown. A quantity of each component in FIGS. 8G-8H are examples only and other quantities of each, or any, component could be used. These foundation blocks are examples only and not limiting of the disclosure. In an implementation, the CGPG GUI 220 may provide additional foundation blocks other than those exemplified herein and/or may provide a subset of the foundation blocks exemplified herein. Each foundation protocol block shown in FIGS. 8G-8H is associated with an operator. The types of entries to the user specification window 506 are determined by the operator.

Referring to FIG. 8G, foundation protocol block 870a shows an example of a refresh operator 892. The specification window 870b for the refresh operator includes the fields comment and parameter name. The refresh operator causes the clinical guidance engine 235 to obtain an updated parameter value from a medical device without generating a clinical guidance UI control. For example, the clinical guidance engine 235 may send a medical device instruction 425 to a medical device in the network 499. The medical device instruction 425 may cause the medical device to send the most recent parameter measurement or reading as collected from the patient to the clinical guidance engine 235. The output port for the refresh operator is the advance port 894 that leads to a subsequent protocol block following the execution of the refresh parameter instruction and as defined by the sequencing link connecting the output port and the input port of the subsequent protocol block.

The refresh operator 892 acquires a new measurement of a patient parameter. When a parameter is used in any of the protocol blocks, the clinical guidance engine 235 automatically acquires the parameter from the communicatively coupled medical devices attached to the patient or the user is provided guidance at the clinical guidance UI 250 on how to acquire the parameter. Once the parameter is acquired, the clinical guidance engine 235 caches the value for subsequent use subject to a pre-determined time limit. The refresh operator 892 causes clinical guidance engine 235 to discard a cached and unexpired value and utilize a new measurement acquired from the medical devices 265.

The protocol 899 illustrates an example of the refresh operator 892 within a protocol. The protocol 899 provides a NIBP monitoring workflow based on non-invasive blood pressure (NIBP) measurements. As a monitoring protocol, the protocol 899 includes a monitor block 87 instead of a start block. When used in an encoded clinical guidance protocol, the monitor block 87 causes the clinical guidance engine 235 to implement the protocol stemming from the monitor block 87 as a monitoring protocol. The clinical guidance engine 235 looks at all of the parameters and/or variables in the monitoring protocol and monitors for changes. Every time the clinical guidance engine 235 receives a new or refreshed value for one of the parameters and/or variables, the clinical guidance engine 235 compares that new or refreshed value to a previously cached value. If there is a change, then the clinical guidance engine 235 implements the monitoring protocol. For example, for the monitoring protocol 899, the clinical guidance engine 235 watches the systolic blood pressure (SBP) and the heart rate (HR). If a change occurs in either or both of these values, the clinical guidance engine 235 implements the protocol 899 as described below. If there is no change, then the clinical guidance engine 235 continues to watch the SBP and HR for changes. In this manner, the encoded clinical guidance protocols 225 enable the clinical guidance engine 235 to monitor patient conditions in the background and bring these conditions to the attention of the caregiver and/or adjust medical device settings only in response to changes. The monitor block 87 is shown in connection with a conditional evaluation block 89c in the example of FIG. 8G. This is an example only and other combinations of protocol blocks with the monitor block 87 are within the scope of this disclosure. As another example, the monitor block 87 could be linked with a sequencing link to a counter block that counts a number of repetitions of a particular activity. The monitoring block 87 enables a protocol that, for example, watches the counter block and then brings up a reminder or alert with every increment of the counter block.

The condition evaluation block 89c evaluates the mean systolic blood pressure (SBP) and heart rate (HR) and provides and instruction to connect NIBP and HR monitors in block 89d if the measurement is unavailable. Depending on the evaluation of the SBP and HR, different timers are set in blocks 89e and 89f. For each of these blocks, when the time expires, the clinical guidance engine 235 implements a refresh operation to check the NIBP value at the end of the timer duration. Additionally, for each of these blocks, the advance operation allows the clinical guidance engine 235 to immediately invoke a subsequent workflow block in an envelope protocol in parallel with the timer countdown. When the time expires, and during operation of the subsequent workflow block, the clinical guidance engine 235 will evaluate the condition evaluation block 89c in order to monitor these parameters in parallel with other protocol steps. If the timers expire, the refresh blocks 89a and 89b cause the clinical guidance engine 235 to request updated NIBP parameter values from the NIBP monitor. The sequencing links 88a and 88b then return to the condition evaluation block to evaluate the refreshed NIBP values.

Referring to FIG. 8H, foundation protocol block 890a shows an example of a repeat operator 891. The specification window 890b for the repeat operator 891 includes the fields comment, number of repetitions 81 and repetition counter variable 82. The repeat operator 891 causes the clinical guidance engine 235 to repeat one or more protocol blocks in a loop. For example, in the protocol section 895, the repeat operator 891 causes a looped repetition of the blocks 897a, 897b, and 897c. The number of repetitions is specified in the field 81. In the example protocol 895, the number of repetitions is set at three. The loop function can truncate and exit if a variable reaches a particular value. In this example, the variable 83, “quality,” is tested in block 897c. If this variable reaches a particular value, then the repetition ends at the end repetition port 80b. In this manner, the repeat operation can prevent a physiological parameter or medical device parameter from reaching an undesired value based on the particular circumstances of a particular patient. If the repetition reaches the number of repetitions specified in field 81, then the repetition ends at the exit port 80c. The next port 80d enables a transition to another section of the protocol via a sequencing link from the next port 80d to one or more subsequent protocol blocks.

The foundation blocks described above correspond to various functional categories. For example, some foundation protocol blocks may cause the clinical guidance engine 235 to generate a clinical guidance UI control and receive information via the UI control. Protocol blocks that generate a clinical guidance UI control may provide multiple screens and multiple types of information in response to a user invocation of the UI control. For example, the medication administration block described below may cause the clinical guidance engine 235 to provide a screen that tells a user to administer a drug. That screen may also include a control that the user may select to obtain additional guidance on drug administration as provided in one or more screens. All of the screens provided at the clinical guidance UI 250 may be provided while the clinical guidance engine 235 executes the corresponding block. Thus, there may not be a one-to-one correspondence between one screen at the clinical guidance UI 250 and the execution of a particular protocol block.

The protocol blocks that include the alert operator 875, the checklist operator 876, the multiple-choice operator 877, the medication administration operator 879, the instruction operator 880, and the UI user input operator 881 are all examples of foundation protocol blocks that execute an operation that generates a UI control at the clinical guidance UI 250. For these protocol blocks, there is caregiver interaction with the clinical guidance engine 235 that is necessary for the execution of the protocol block. The caregiver may receive information from the clinical guidance engine 235 via the clinical guidance UI 250 (e.g., the caregiver guidance 435) and/or may provide information to the clinical guidance engine 235 via the clinical guidance UI 250 (e.g., the caregiver input 430). Although shown schematically herein as guidance and input provided at a display, this guidance and input may be in the form of audible guidance, audible input, haptic guidance, haptic input, and/or guidance and/or input from a virtual display depending on the type of device that provides the clinical guidance UI 250.

As another example, other foundation protocol blocks may cause the clinical guidance engine 235 to execute at least one operation as a background task without generating a clinical guidance UI control. For example, the protocol blocks that include the condition evaluation operator 528, the range evaluation operator 870, the value assignment operator 871, the timer operator 872, the delay operator 873, the computation operator 874, the trend operator 878, the refresh operator 892, and the repeat operator 891 are all examples of foundation protocol blocks that may execute at least one operation as a background task. For these protocol blocks, there may be no caregiver interaction with the clinical guidance engine 235 that is necessary for the execution of the protocol block. The caregiver may not even be aware that the clinical guidance engine 235 is executing these protocol blocks. The operation executed by these protocol blocks is defined by the operator and the entries to the user specification window 506 that corresponds to the operator.

For example, the condition evaluation operator 528 corresponds to a foundation protocol block that executes as a background task without generating a control at the clinical guidance UI 250. The operation of the condition evaluation protocol block may be specified as [(SpO2>90) AND (EtCO2>35)]. In such an example, the condition evaluation protocol block causes the clinical guidance engine 235 to evaluate a pulse oximetry signal and a CO2 signal received at the clinical guidance engine 235 from a medical device 265. The function of the condition evaluation protocol block is to evaluate these signals. If the evaluation determines that [(SpO2>90) AND (EtCO2>35)], the clinical guidance engine 235 moves to a protocol block sequenced to a logically true output port of the condition evaluation initiation block, else the clinical guidance engine moves to a protocol block sequenced to a logically false output port of the condition evaluation protocol block.

The operators perform various general functions. For example, the condition evaluation operator 528 and the range evaluation operator 870 are examples of evaluation operators that cause the clinical guidance engine 235 to perform an evaluation. As another example, the timer operator 872 and the refresh operator 892 are examples of command operators that function to command the clinical guidance engine 235 to perform a task. As a further example, operators like the value assignment operator 871, the delay operator 873, the computation operator 874, the trend operator 878, and the repeat operator 891 are examples of internal logic operators that cause the clinical guidance engine 235 to execute the programming logic of the protocol according to the operations performed by these operators. In some examples, internal logic protocol blocks with internal logic operators may not cause interactions with any external devices. The internal logic may also include defining values for parameters and/or variables.

In some examples, the foundation protocol blocks that cause the clinical guidance engine 235 to execute an operation as a background task without generating a clinical guidance UI control function as initiation blocks, or background triggers, for a protocol. A single block or a combination of two or more blocks may function as the background trigger. As examples, these blocks may include the condition evaluation operator 528, the range evaluation operator 870, the value assignment operator 871, the timer operator 872, the delay operator 873, the computation operator 874, the trend operator 878, and/or the refresh operator 892. Instances of these blocks may be tagged to instruct the clinical guidance engine 235 to execute code that repeatedly evaluates a variable and/or parameter invoked by the background trigger block in order to provide a trigger function. The clinical guidance engine 235 repetitively evaluates the variable and/or parameter and flags a change in that variable and/or parameter. The variable and/or parameter may base on an input to the clinical guidance engine 235 from the medical devices 265 and/or based on an input to the clinical guidance engine from the clinical guidance UI 250. The clinical guidance engine 235 then implements the background trigger block and this block will trigger a subsequent protocol block and/or workflow when a condition within the background trigger block is satisfied due to the change in the evaluated parameter and/or variable. If a condition a variable and/or parameter invoked continues to implement one or more of these blocks repeatedly until a block condition is satisfied and/or until a change in a parameter and/or variable. The initiation blocks, or background triggers, execute in the background of other activities of the clinical guidance engine 235 and then initiate, or trigger, the execution of a subsequent protocol block. In some examples, the subsequent protocol block may be a workflow block. In an implementation, a generated protocol may include an initiation block and a workflow block. Such a protocol enables the clinical guidance engine 235 to periodically evaluate a variable and/or parameter and a condition specified in the initiation block and then execute the workflow block when the condition is satisfied.

For example, an ARF workflow block may link to a background trigger block that monitors EtCO2 and SpO2 values and instructs the clinical guidance engine 235 to implement the ARF workflow if these monitored values correspond to particular pre-determined values or ranges. The clinical guidance engine 235 may watch for these trigger elements or conditions and invoke the generated protocol when the trigger elements or conditions occur or are satisfied. In an implementation, the clinical guidance engine 235 may concurrently evaluate multiple background trigger blocks associated with the multiple clinical guidance protocols. This evaluation occurs in the background without generating UI controls at the clinical guidance UI 250. For example, the multiple initiation blocks may evaluate breathing conditions and heart conditions. The clinical guidance engine 235 may then implement one or more clinical guidance protocols based on satisfaction of the evaluation of one or more initiation blocks. If more than one condition is implicated (e.g., a breathing problem co-occurring with a heart problem), the clinical guidance engine 235 may provide a caregiver option at the clinical guidance UI 250 to select the priority of care. In an implementation, the triggered protocols may include priority determination blocks that evaluate parameters indicative of the priority of one set of interventions over another and branch between protocols based on these priorities. The initiation blocks are computational in nature and do not require caregiver interaction or attention. In an implementation, an initiated protocol may include internal evaluation blocks that enable the clinical guidance engine 235 to exit the protocol or rearrange protocol steps based on the evaluation. In an implementation, an initiation block may cause the clinical guidance engine 235 to pre-empt an ongoing protocol due to the triggering of a higher priority protocol. The clinical guidance engine 235 may hold a place in the lower priority protocol and resume at a later time when the higher priority issue is resolved. For example, during a protocol to evaluate a patient for spinal cord injury, a cardiac arrest protocol may be initiated. A cardiac arrest poses an immediate threat to the life of a patient and would take priority. In an implementation, a protocol may include a workflow performance operation that directs the clinical guidance engine 235 to branch to a particular protocol if an evaluation attached to the workflow performance operation is satisfied.

In an implementation, an envelope protocol may call multiple constituent protocols in a prioritized order such that the higher-level protocol may move from a lower priority protocol to a higher priority protocol based on physiological monitoring triggers. Alternatively, the envelope protocol may include two or more constituent protocols and run these as multi-threaded protocols. With multi-threaded protocols, any of the two or more constituent protocols can be activated based on satisfaction of a triggering condition. The generated protocol 225 may enable the clinical guidance engine 235 to stack and run multiple active protocols in a multi-threaded fashion, where all threads can be active and advanced simultaneously either by multiple users or by the system and a user. In an implementation, the clinical guidance engine 235 may stack multithreaded protocols and prioritize execution according to medical direction and therefore utilize different areas of the clinical guidance UI 250. For example, the main protocol UI area may be populated by a ventilation instruction while a HR trigger enables a cardiac arrest protocol that then moves to the main window and moves the ventilation protocol step to the stacked alert area to be followed later either by the user concluding the cardiac arrest protocol or by the user accessing the alert box and re-enabling that protocol. In an implementation, a background trigger block in a multi-threaded protocol may generate an alert for the user at the clinical guidance UI 250. The alert may notify the user of a priority selection by the clinical guidance engine 235 or may request a priority selection by the user. In this way, the user also has control over prioritization of the multiple threads when they are active by selecting workflow that is primarily provided at the clinical guidance UI 250.

As an example of another type of protocol block function, in an implementation, one or more protocol blocks may be medical device interaction protocol blocks configured to cause the clinical guidance engine 235 to interact with the medical devices 265. For example, the medical device interaction protocol blocks may include the refresh block 870a, the UI instruction block 860a, the condition evaluation block 810a, the range evaluation block 805a, and/or the alert block 835a. The medical device interaction protocol blocks may utilize device drivers to either provide instructions to the medical devices and/or to retrieve information from the medical devices. For example, the retrieved information may include data such as a patient status parameter or a device status parameter. Further the medical device interaction protocol blocks may cause the clinical guidance engine 235 to verify the medical devices that are communicatively coupled to the clinical guidance engine 235 during a patient encounter where the engine 235 is providing caregiver guidance in real-time. The clinical guidance engine 235 may then initiate communications with a particular medical device based on this information and/or search for a communicatively coupled device. In this manner, the clinical guidance engine 235 may tailor the guidance to medical devices actually in use and capable of communications with the engine 235. In various examples, the medical device interaction protocol blocks may cause the clinical guidance engine 235 to provide an instruction (e.g., the medical device instructions 425) to at least one medical device 265. These instructions may include an instruction to at least one medical device to perform one or more of a therapeutic or monitoring function. The therapeutic function may include providing a therapeutic treatment or changing a therapeutic treatment parameter. The monitoring function may include initiating and/or continuing monitoring of a physiological parameter of the patient by requesting data from the medical device. In some examples, the clinical guidance engine 235 may monitor the physiological parameter of the patient for a pre-determined time interval. In an implementation, the clinical guidance engine 235 may provide closed loop control instructions to the medical device 265. As discussed above, some of the protocol blocks may receive and evaluate data from at least one medical device without generating a clinical guidance UI control. The clinical guidance engine 235 may retrieve information from the resource manager 240, 260, and/or 280 for this evaluation. In an implementation, the clinical guidance engine 235 may provide caregiver guidance for at least one medical device at the clinical guidance UI based on the information from the resource manager 240, 260, and/or 280. The user specifications for the medical device interaction protocol blocks may include a user specification of a type of data to retrieve from and/or send to the medical device, a type of medical device. The user specification of the type of medical device may include a make and model of the medical device. Alternatively, the user specification may indicate a functional type of device, such as defibrillator or ventilator, and the clinical guidance engine 235 may implement the protocol block by retrieving more specific information, such as make and model, from the resource manager 240, 260, and/or 280.

Any or all of the foundation protocol blocks that utilize a user specification of a physiological parameter evaluation may cause the clinical guidance engine 235 to retrieve physiological parameter information from the resource manager 240, 260, and/or 280. The clinical guidance engine 235 may then present this information at the clinical guidance UI 250 in the form of user guidance, verification of physiological information entered by the user, alerts, etc.

In order to build and generate a protocol, the user of the CGPG GUI 220 may select at least one foundation block and at least one workflow block. The foundation block may function as an initiation block for the workflow block. As selected from the menu 502, the initiation block is an unspecified initiation block. As discussed with regard to FIG. 6, highlighting or clicking on the initiation block in the display area 504 pulls up a specification window 506 that corresponds in format to the highlighted initiation block. The user of the CGPG GUI 220 provides a user specification to the specification window 506. The CGPG engine 210 is configured to incorporate the user specification into the unspecified initiation block to convert the unspecified initiation block to a specified initiation block. This specified initiation block is then configured to cause the clinical guidance engine to execute the at least one workflow block based on the specific operation defined in the specified initiation block.

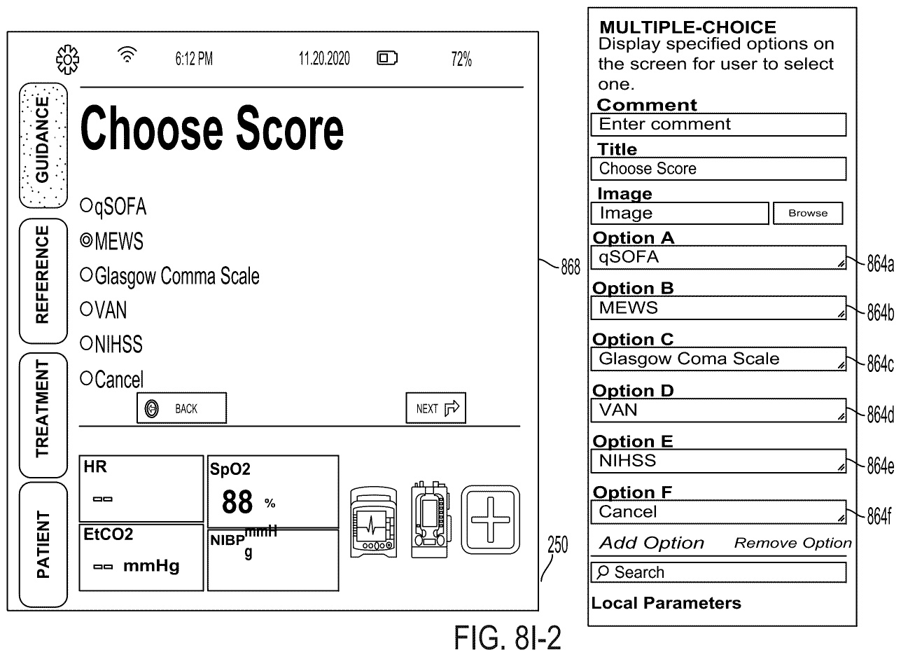

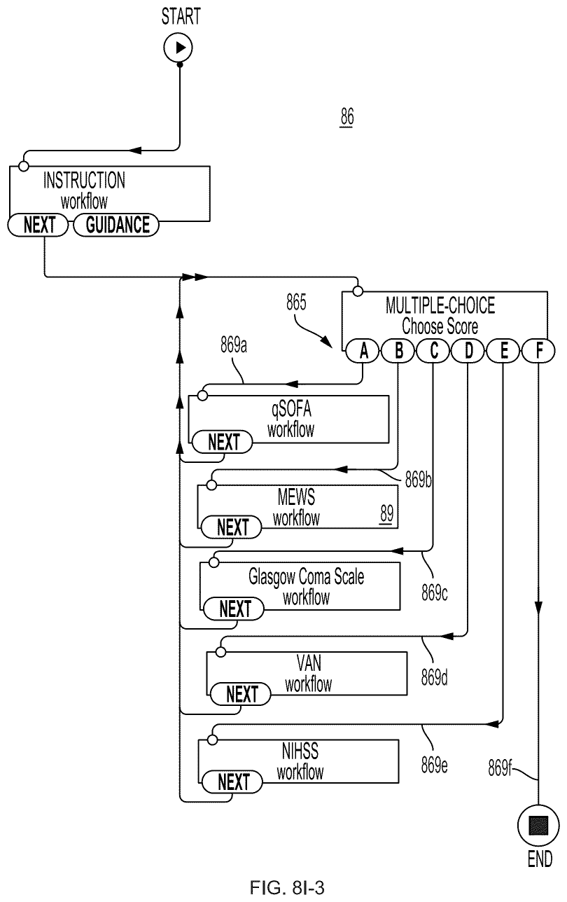

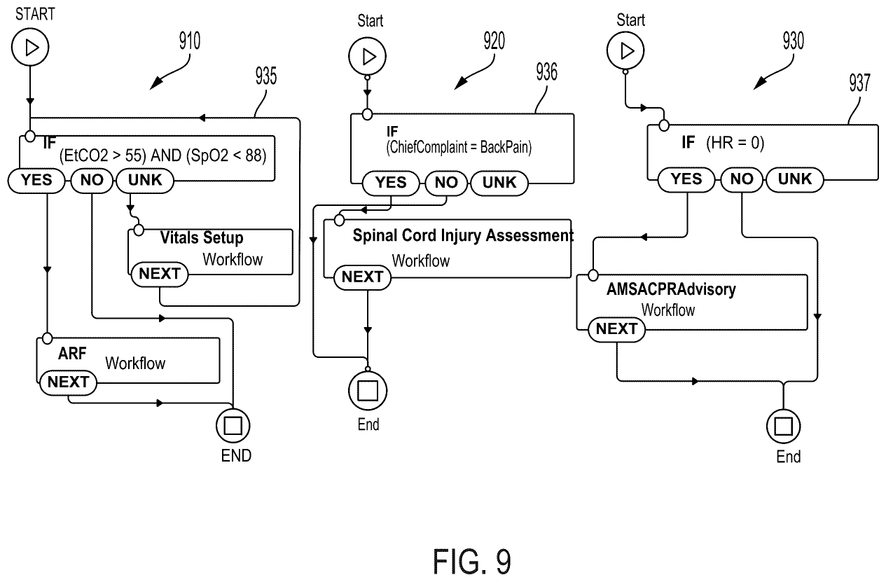

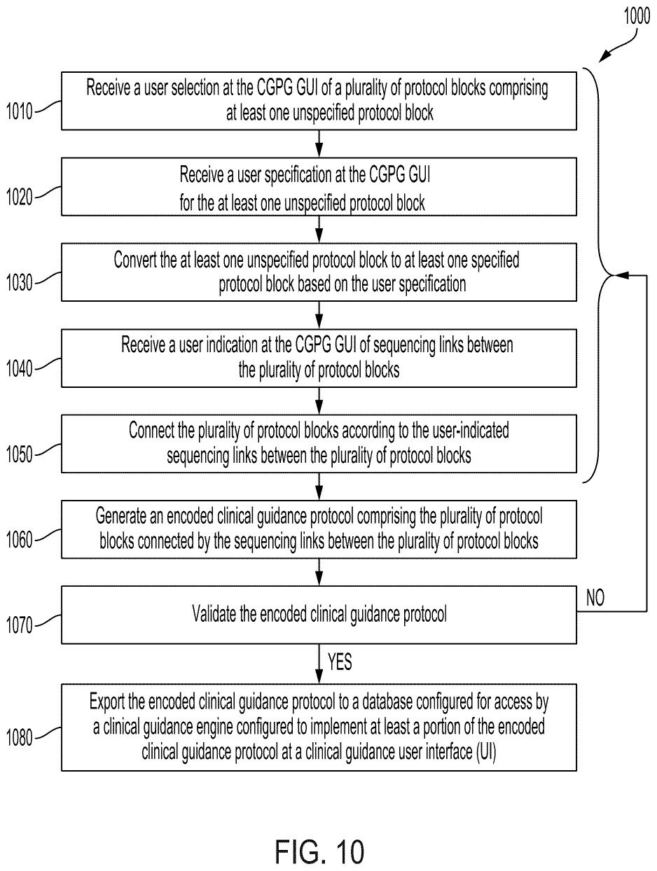

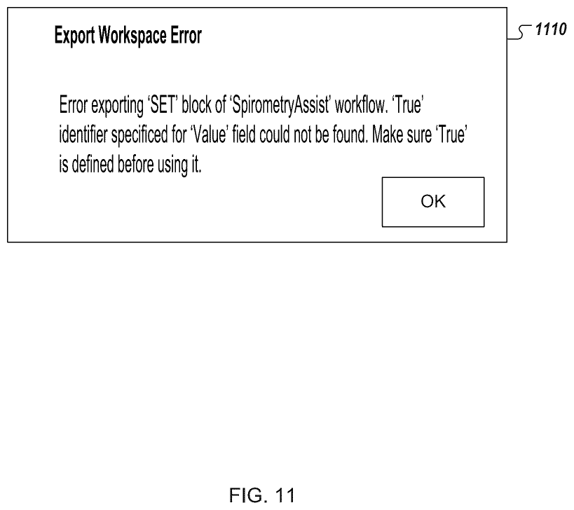

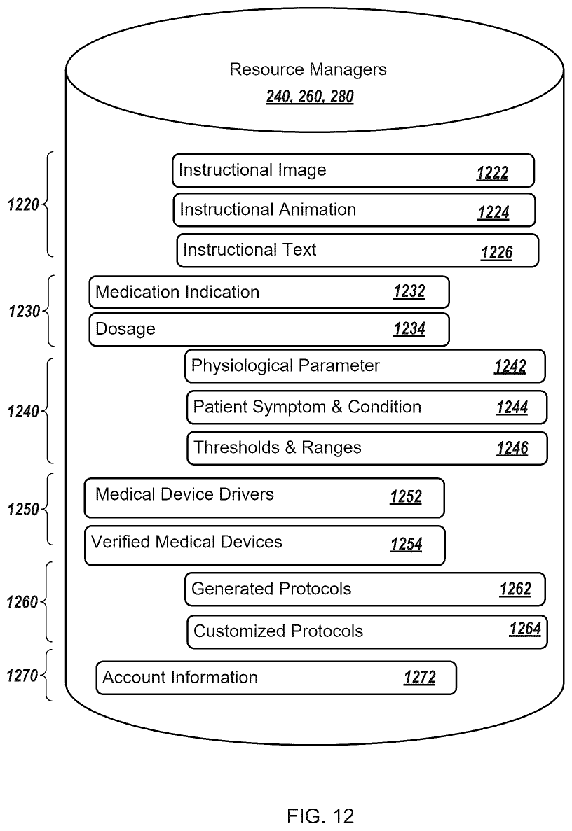

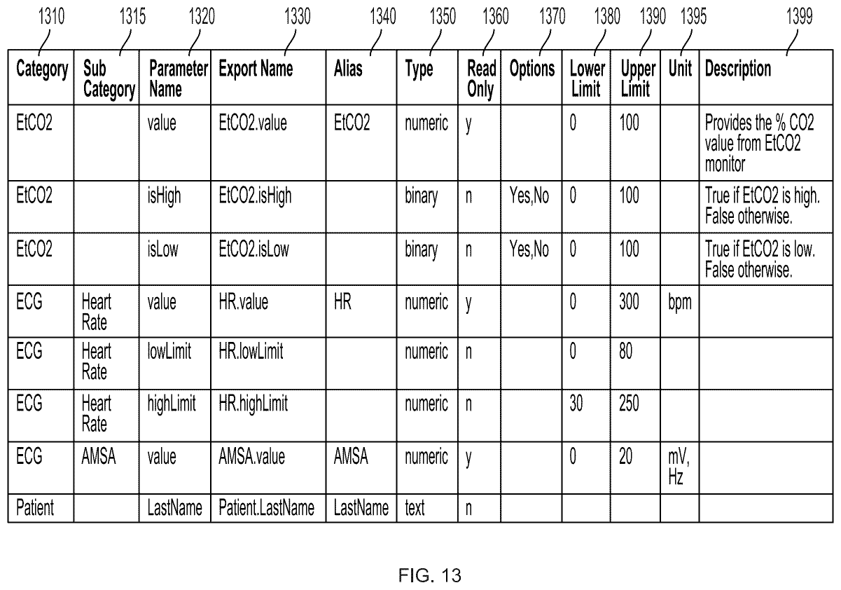

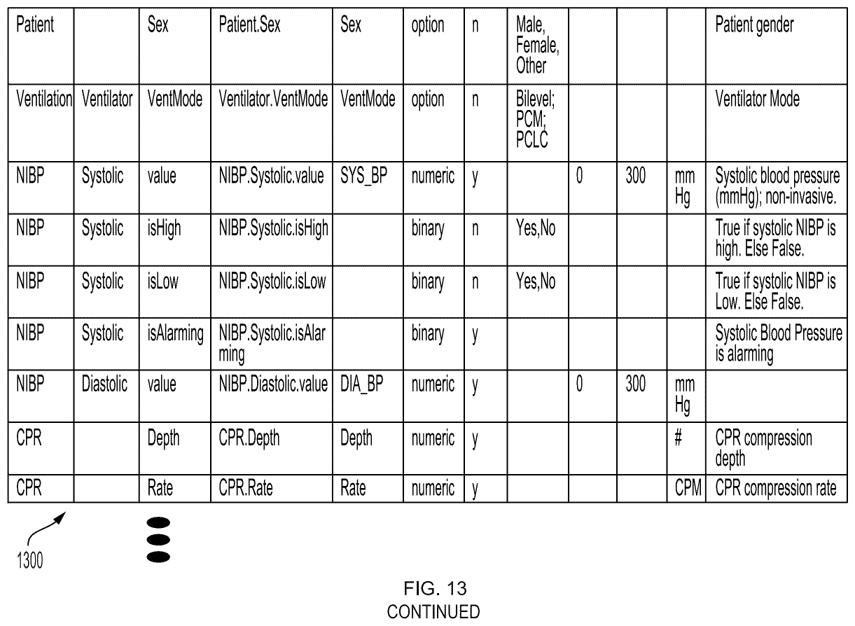

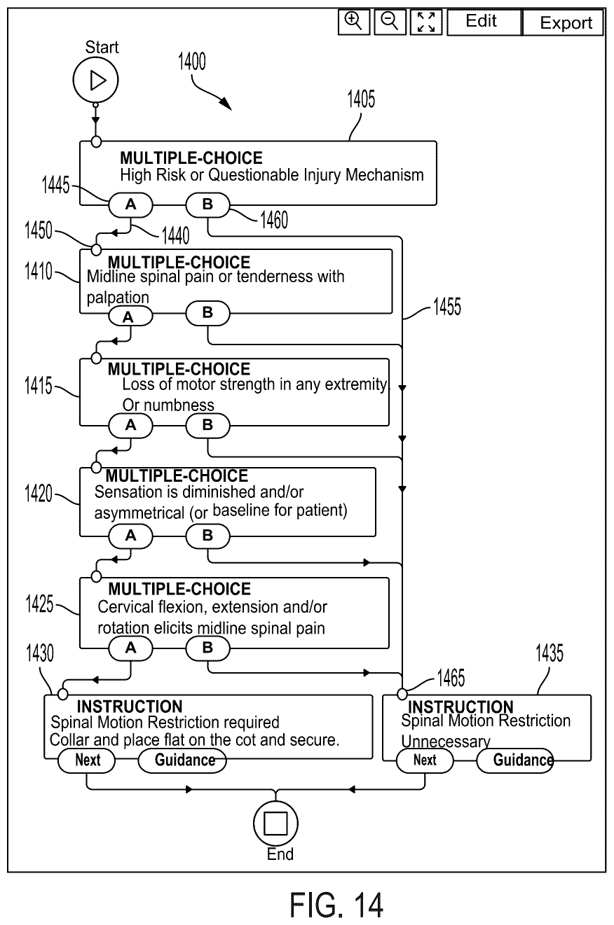

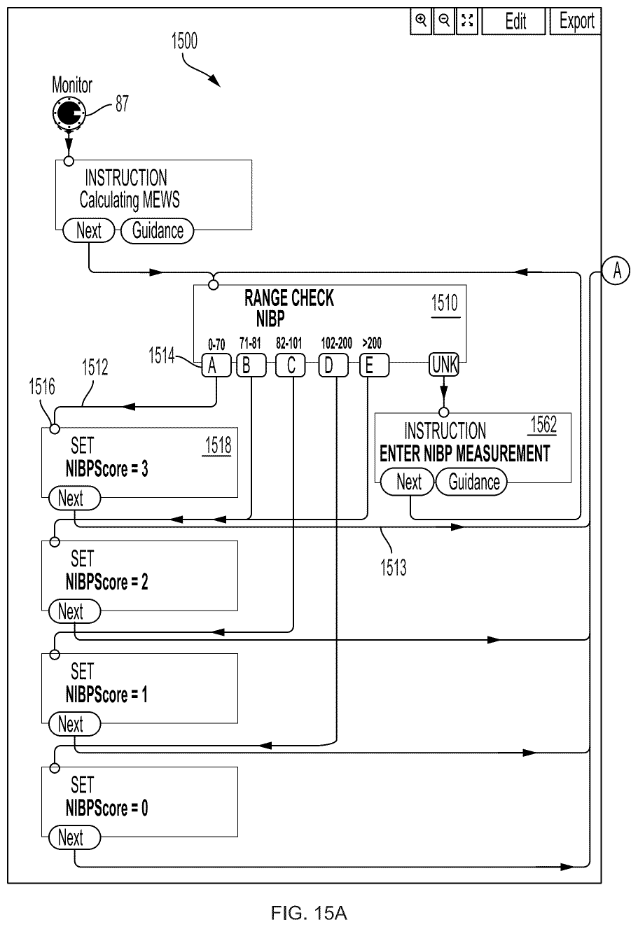

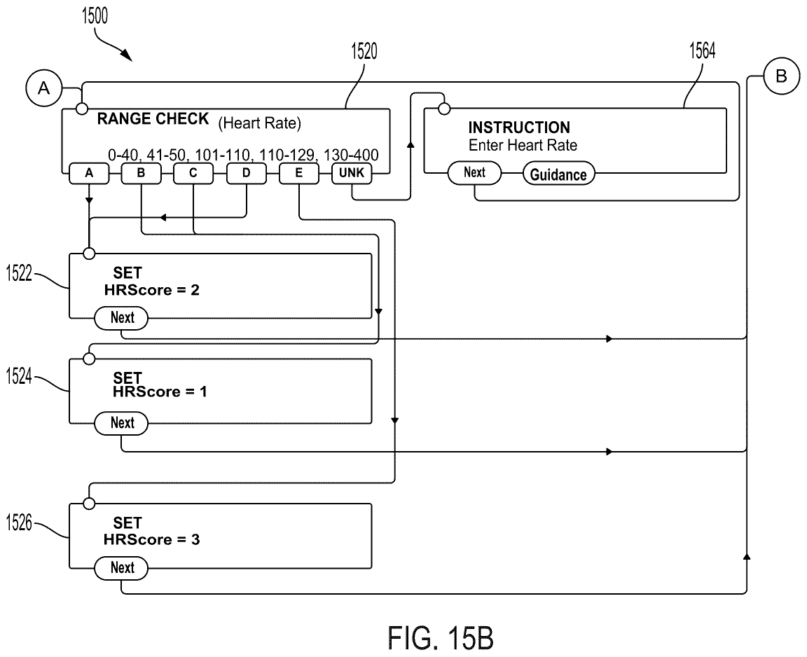

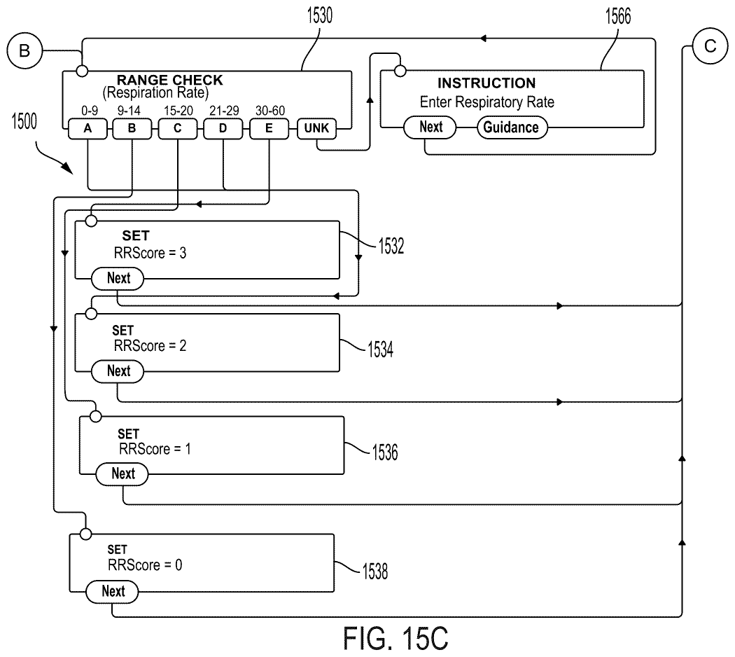

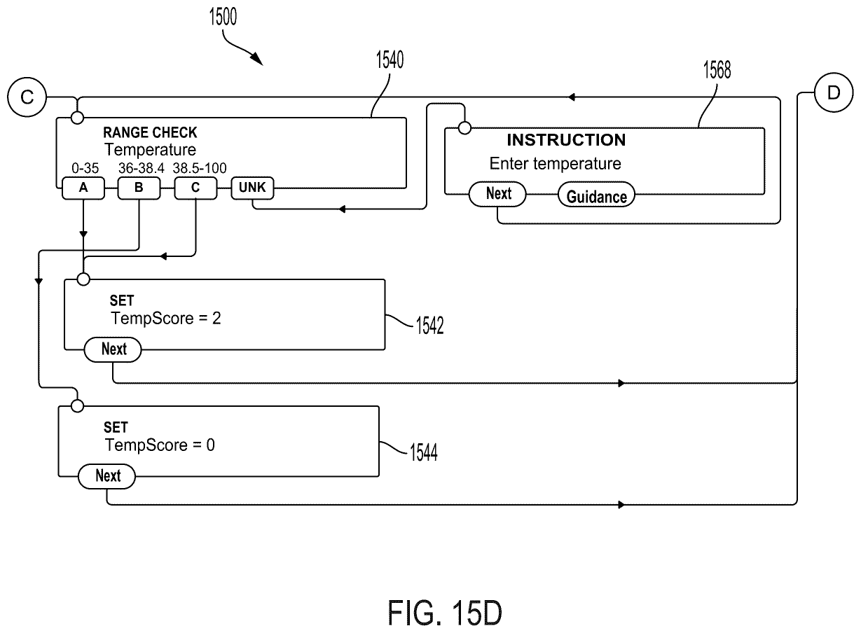

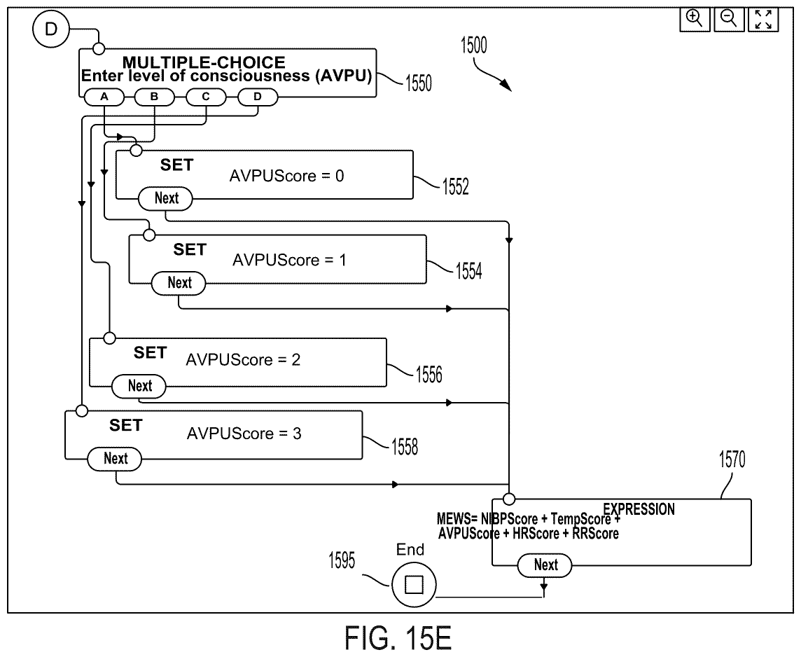

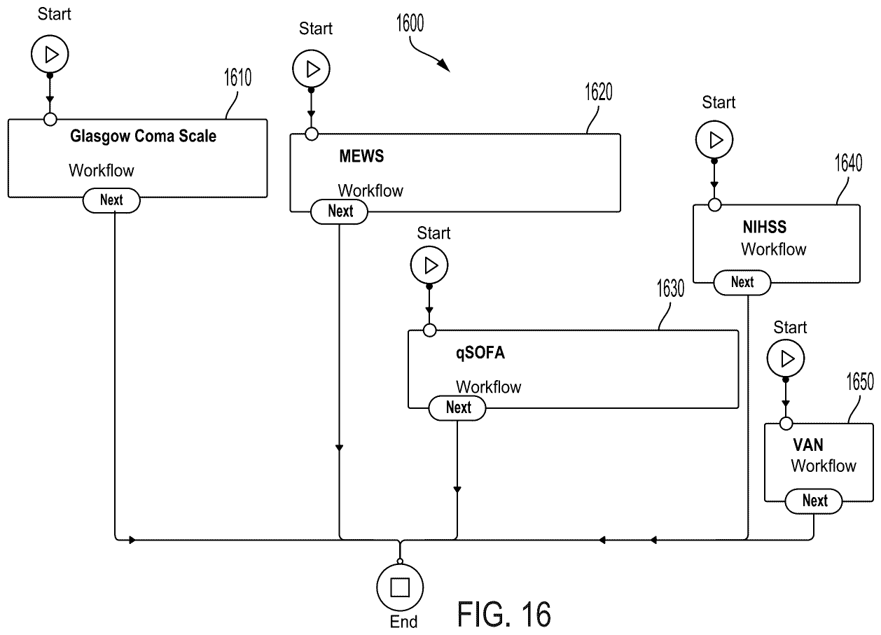

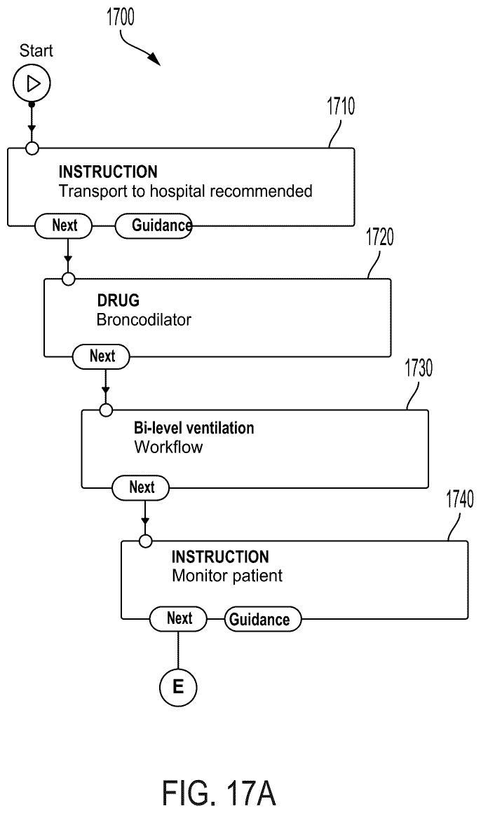

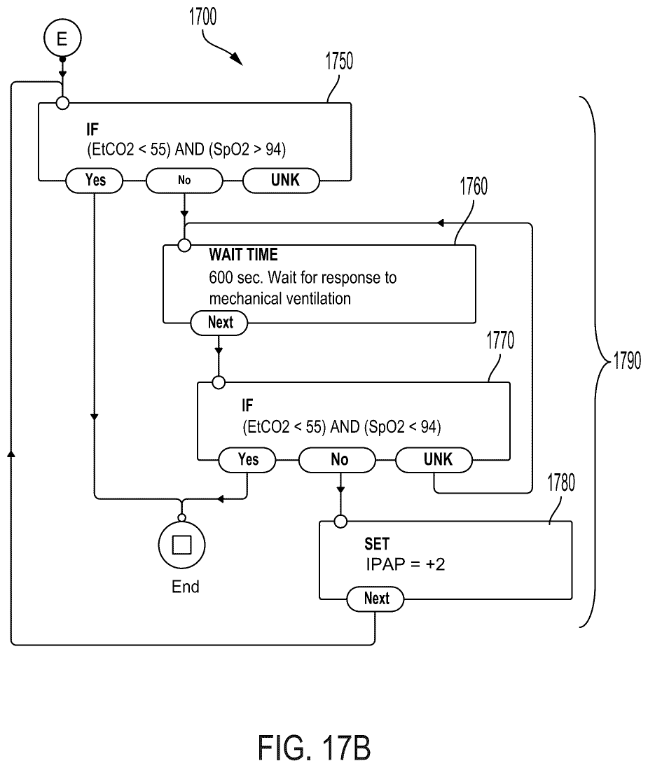

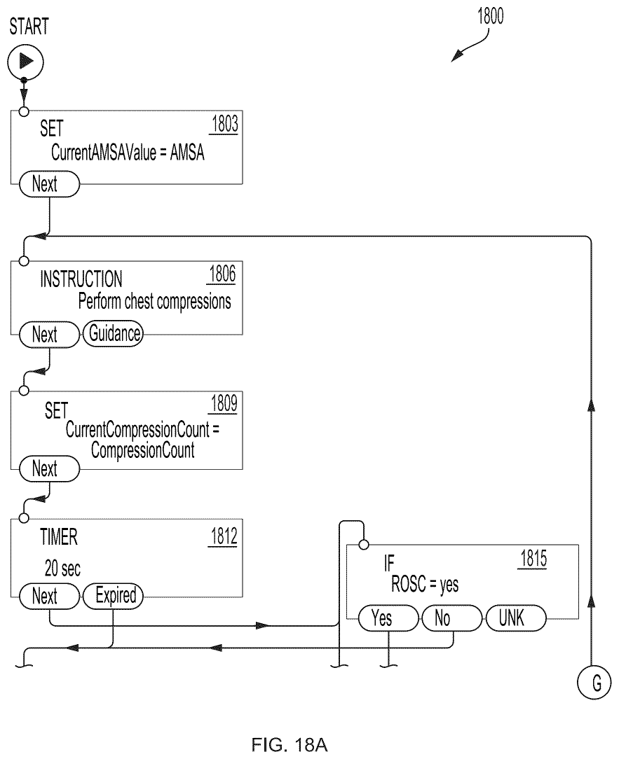

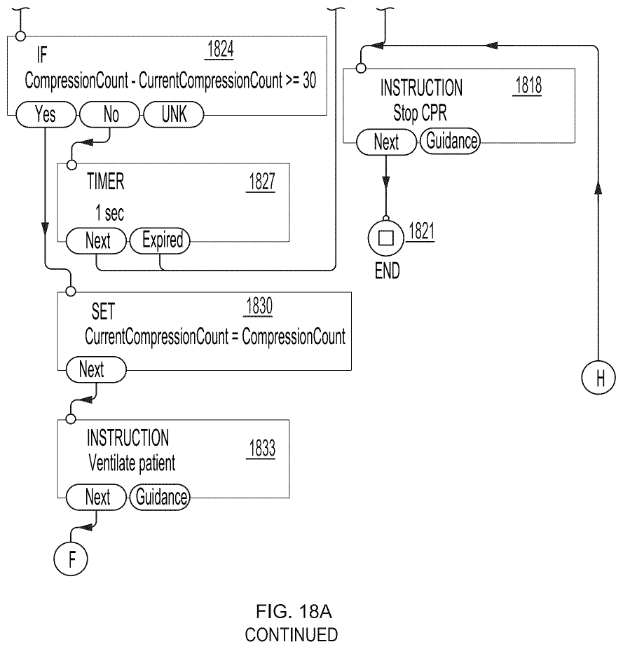

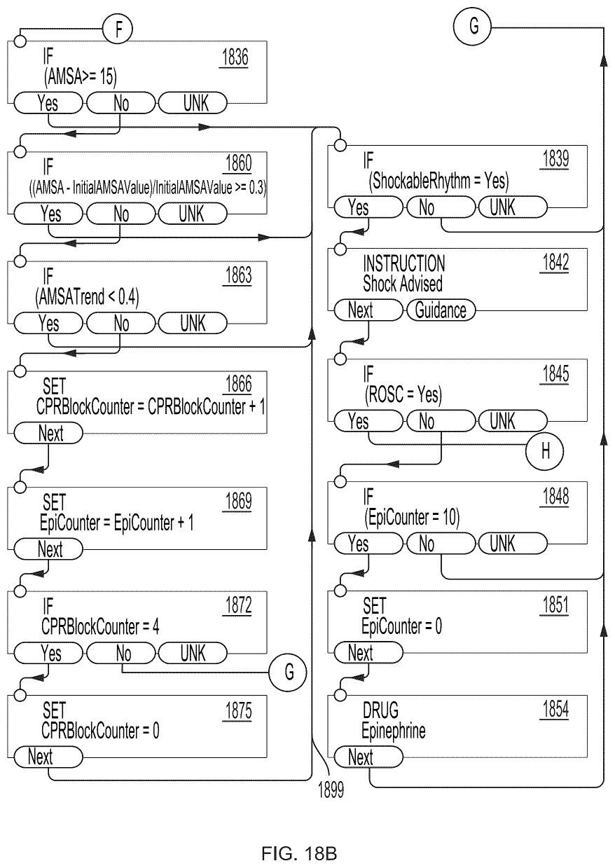

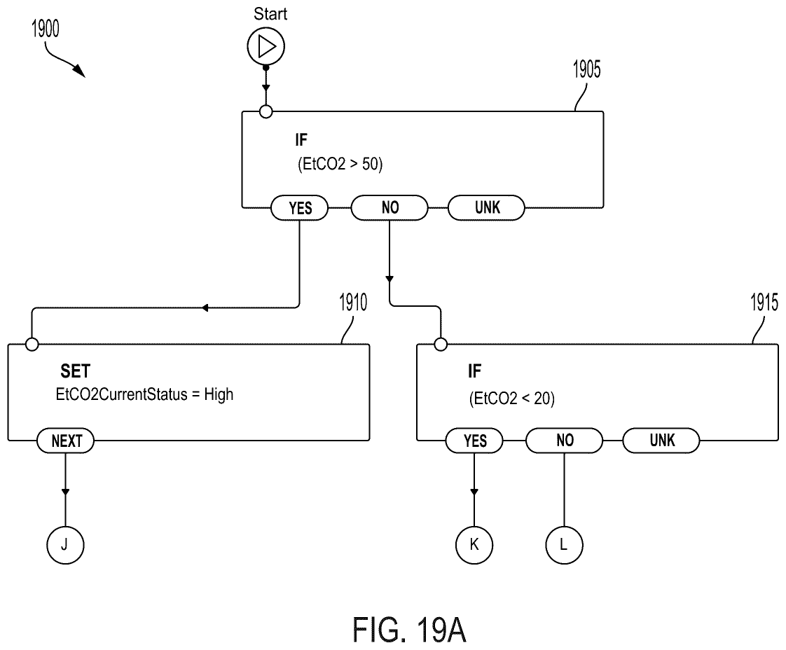

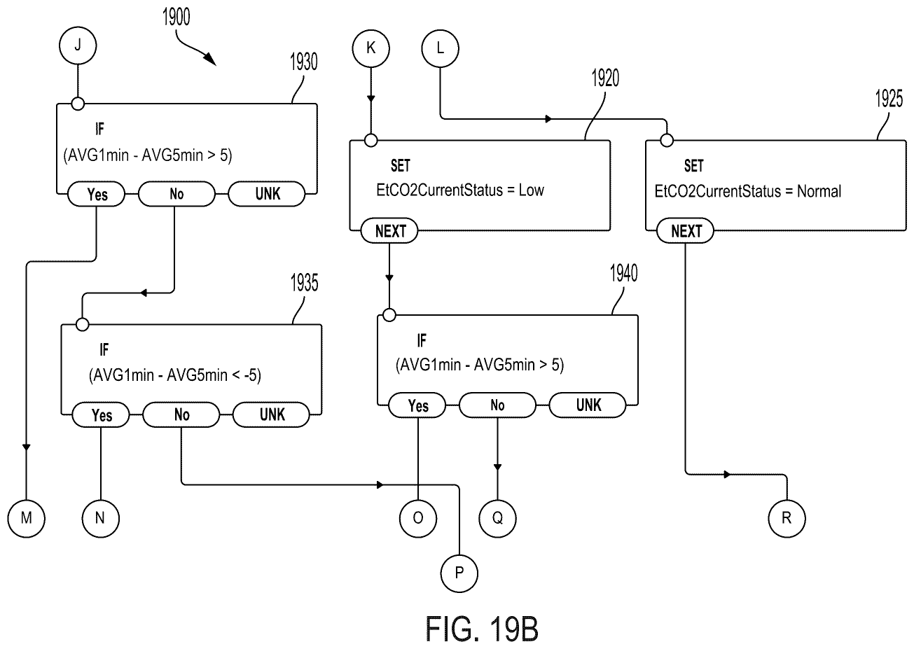

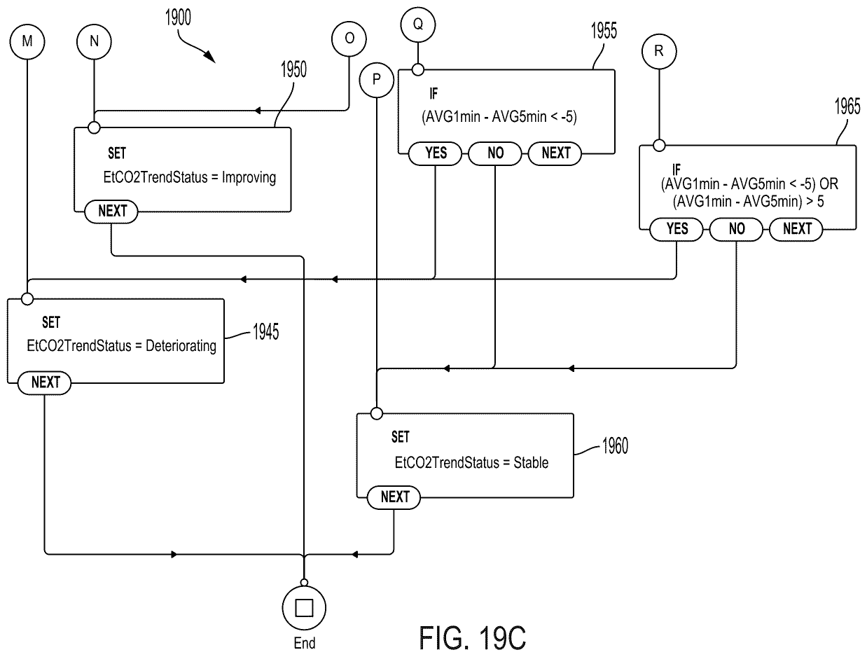

Referring to FIGS. 81-1-81-3, examples of clinical guidance UI features corresponding to encoded clinical guidance protocol specifications are shown. FIG. 8I-1 shows clinical guidance UI features 848 for the clinical guidance UI 250 that correspond to the workflow block 849 according to the user specifications 847a, 847b, 847c, and 847d. These user specifications determine the title 844a, or instruction, that appears on the clinical guidance UI 250. The parameter options 847b, 847c, and 847d appear on the UI 250 as selectable options 844b in a check list. Each of these options is associated with a parameter, e.g., the parameters 843a, 843b, and 843c. In this example, when a user selects “shortness of breath” and “hypotension” and does not select “hypertension,” the parameters “shortb” and “hypoT” are set to a logically true value and the parameter “hyperT” is set to a logically false value. A user of the CGPG GUI 220 may embed the encoded check list protocol 84 within a larger protocol and link it to a subsequent protocol or workflow blocks. The check list protocol 84 will pass the parameters 843a, 843b, and 843c to the subsequent protocol or workflow blocks and these blocks may perform operations based on evaluations of these parameter values. FIGS. 81-2 and 81-3 show an example of UI features 868 at the clinical guidance UI 250 for a multiple-choice workflow 86. In this example, the fillable fields 864a, 864b, 864c, 864d, 864e, 864f allow the user of the CGPG GUI 220 to specify options associated with each of six output ports. The sequencing links 869a, 869b, 869c, 869d, 869e, and 869f link each of the options 865 to a corresponding scoring workflow. For example, the link 869b links the output port “B” with a MEWS workflow, an example of which is provided in FIGS. 15A-15E. When the user of the clinical guidance UI 250 selects “MEWS,” the output port “B” is set to a logically true value and the encoded clinical guidance protocol 89 proceeds to the MEWS workflow block 89 based on the sequencing link address uniquely identifying the MEWS workflow block 89.

Referring to FIG. 9, an example of parallel processing of multiple initiation blocks by the clinical guidance engine is shown. A quantity of each component in FIG. 9 is an example only and other quantities of each, or any, component could be used. The clinical guidance engine 235 may evaluate multiple initiation blocks in parallel to track the clinical context of the patient. As illustrated in FIG. 9, the clinical guidance engine 235 may load the three workflows 910, 920, and 930 to provide guidance for potential respiratory conditions, spinal cord injury conditions, and cardiac conditions of a patient. The clinical guidance engine 235 may then execute the appropriate workflow block triggered by a corresponding initiation block. For example, the clinical guidance engine 235 may monitor physiological parameter signals coming from the medical devices 265 and may monitor entries to a patient chart 455. In the example of FIG. 9, the signals may include signals from a patient monitor or patient monitor/defibrillator such as heart rate (HR), end tidal CO2 (EtCO2), and oxygen saturation (SpO2). One of the entry fields for the patient chart may be a chief complaint of the patient. The clinical guidance engine 235 may evaluate whether HR, EtCO2, and SpO2 satisfy any of the conditions in background initiation block 935 of workflow 910, background initiation block 936 of workflow 920, or background initiation block 937 of workflow 930. Additionally, the clinical guidance engine 235 may watch for an entry to a chief complaint field of an electronic patient chart of “back pain” or text with a similar meaning. If one of these conditions is met, the clinical guidance engine 235 implements the sequenced workflow. If two or more of these conditions is met, the clinical guidance engine 235 may determine a priority or query a caregiver for a priority. For example, for the three workflows exemplified in FIG. 9, cardiac arrest workflow 930 may have top priority followed by the respiratory distress workflow 910 followed by spinal assessment workflow 920. The clinical guidance engine 235 and/or the caregiver may assign these priorities based on the relative threat to the life of the victim (e.g., a heart rate of zero is the most life threatening followed by acute respiratory failure followed by a spinal cord injury). Where multiple protocols, or workflows, are invoked, the clinical guidance engine 235 may reorder the priority based on changes in response to interventions and/or changes in the patient state. For example, a patient may have a non-zero heart rate restored and an alleviation of respiratory distress thus causing the spinal cord injury to be top priority. As another example, a patient may initially present with back pain without an indication of cardiac arrest (e.g., HR>0). The clinical guidance engine 235 may initiate the workflow 920. However, the patient may go into cardiac arrest and, in response, the change in patient state may cause the clinical guidance engine 235 to implement workflow 930 and reduce the priority of workflow 920 at least temporarily.

Referring to FIG. 10, an example of a method of generating an encoded clinical guidance protocol is shown. The method 1000 is, however, an example only and not limiting. The method 500 can be altered, e.g., by having stages added, removed, rearranged, combined, and/or performed concurrently. For example, the CGPG engine 210 may perform the method 500.

At the stage 1010, the CGPG engine 210 may receive a user selection at the CGPG GUI 220 of a plurality of protocol blocks. The plurality of protocol blocks may include at least one unspecified protocol block. The unspecified protocol block may be an unspecified foundation block. The unspecified foundation blocks may be sequenced with one another to generate a workflow block. The workflow block may include a plurality of foundation blocks and/or a combination of one or more foundation blocks and one or more other workflow blocks. Workflow blocks may be protocol blocks built-in to the CGPG GUI 220 and/or may be protocol blocks created by a current user of the CGPG GUI 220, stored for use in a protocol, and imported from a resource manager for use at the CGPG GUI 220. The workflow block may be associated with a specific clinical procedure and/or a specific patient condition. In an implementation, the plurality of protocol blocks may include at least one unspecified foundation block and at least one workflow block. The unspecified foundation block may be sequenced with a workflow block and function as an initiation block for the workflow block. The initiation block defines an operation that the clinical guidance engine 235 must execute prior to executing the workflow block. The initiation block may trigger execution of the linked workflow block.

At the stage 1020, the CGPG engine 210 may receive a user specification at the CGPG GUI 220 for the at least one unspecified protocol block. The CGPG engine 210 may be configured to provide the user specification window in a format based on the user selection of the unspecified protocol block. For example, in FIG. 5B, the user specification window 506 is provided in a format for a condition evaluation operation that corresponds to the condition evaluation operator 528. The CGPG engine 210 may be configured to receive the user specifications as entries to the user specification window. The user of the CGPG GUI 220 may provide specifications as entries to fillable fields. For example, as illustrated in FIG. 6, the CGPG GUI 220 may capture a user entry of “SpO2” for the “left hand side” fillable field of a specification window for a condition evaluation operation.

At the stage 1030, the CGPG engine 210 may convert the at least one unspecified protocol block to at least one specified protocol block based on the user specification. Each unspecified protocol block may be converted to a specified protocol block by incorporating a user specification provided at the CGPG GUI 220. The specified protocol block may be a specified foundation block resulting from a conversion of an unspecified foundation block. For example, as shown in FIG. 6, the unspecified block 605 has an undefined operation. However, once the fillable fields in the specification window are complete, the CGPG engine 210 converts the unspecified block 605 to a specified block 610. The CGPG engine 210 incorporates the user specification as indicated by entries to the specification window for a particular operator into the unspecified initiation block to convert from an unspecified block to a specified block. The specified initiation block provides an instruction for the clinical guidance engine 235 to execute the block according to the defined operation. For example, the specified initiation block shown in FIG. 6 causes the clinical guidance engine 235 to evaluate whether the condition (SpO2>90) AND (EtCO2>=30) is satisfied.

At the stage 1040, the CGPG engine 210 may receive a user indication at the CGPG GUI 220 of sequencing links between the plurality of protocol blocks. As shown in FIG. 7, the user of the CGPG GUI 220 may provide an indication of sequencing links that connect each of one or more output ports to an input port of a subsequent protocol block. The first protocol block in a sequence may be linked to a start block and the last protocol block in a sequence may be linked to an end block. The output ports are each associated with a specific attribute as described in detail above, for example, in regard to FIGS. 8A-8H. As illustrated schematically in FIG. 7, the CGPG GUI 220 is configured to capture user indications of sequencing links via touchscreen gestures, mouse-driven input, etc. For example, clicking on an output port may cause the CGPG GUI 220 to display a line that the user can drag to a selected input port to indicate the sequencing link between two protocol blocks. The CGPG GUI 220 is configured to display graphic representations of the user indication of the at least one sequencing link in the graphic protocol display area 615. For example, as shown in FIG. 7, the CGPG GUI 220 may display the sequencing link 794 or 796. A user may click on the link and/or a pair of endpoint ports (e.g., the ports 714 and 722 or the ports 716 and 732) and move the sequencing link to modify the designated protocol progression.

At the stage 1050, the CGPG engine 210 may connect the plurality of protocol blocks according to the user-indicated sequencing links between the plurality of protocol blocks. To generate the encoded clinical guidance protocol, every protocol block must be linked to at least one other protocol block.

At the stage 1060, the CGPG engine 210 may generate the encoded clinical guidance protocol 225. For example, the CGPG engine 210 may receive a user selection of the save control 53b (e.g., as shown in FIG. 5A) as an indication that a protocol is generated and complete. The CGPG engine 210 may save the generated protocol to a platform resource manager.

The encoded clinical guidance protocol 225 may include the plurality of protocol blocks connected by the sequencing links between the plurality of protocol blocks. The plurality of protocol blocks in the encoded clinical guidance protocol 225 includes the specified protocol blocks (e.g., the specified foundation blocks and/or the specified initiation blocks). In an implementation, the encoded clinical guidance protocol 225 includes at least one specified foundation block and at least one workflow block. The at least one specified foundation block may include at least one specified initiation block that triggers execution of the at least one workflow block. In an implementation, the CGPG engine 210 may provide the generated protocol as a workflow element in the workflow block menu 515. In this manner, the generated protocol may be included as a sub-protocol in subsequently generated envelope protocol.

At the stage 1070, the CGPG engine 210 may validate the encoded clinical guidance protocol 225. If the encoded clinical guidance protocol 225 satisfies the validation, the method 1000 moves to the stage 1080. However, if the encoded clinical guidance protocol fails to satisfy the validation, the CGPG engine 210 may provide for an invalidation notification and a corrective action. For example, the CGPG engine 210 may provide an error message (e.g., the error message 1110 shown in FIG. 11) that provides an indication of the validation error and corrective actions. The CGPG engine 210 may provide the error message at the CGPG GUI 220. In an implementation, the CGPG engine 210 may report one error at a time and then look for an export request to repeat the validation. Alternatively, the CGPG engine 210 may provide a batch report of all errors found during the validation. In an implementation, the CGPG engine 210 may perform validations as possible in real-time during generation of the encoded clinical guidance protocol 225. The CGPG engine 210 may provide error messages and may provide corrective actions as the user populates the graphic protocol display area 504. In an implementation, the CGPG engine 210 may provide a GUI control that enables a user to request a validation and report at any time prior to an automated validation at export.

In response to an invalidation notification, the method 1000 may return to at least one of the stages 1010, 1020, 1030, 1040, 1050, and 1060 based on a user action in response to the error message. These stages enable a correction of the unvalidated encoded clinical guidance protocol. The CGPG engine may enable the user of the CGPG GUI to return to at least one of selecting the plurality of protocol blocks, specifying at least one unspecified protocol block, or indicating a sequencing link. Alternatively, in an implementation, if the validation fails, the CGPG engine 210 may automatically modify a user specification and/or a sequencing link for an unvalidated clinical guidance protocol. The CGPG engine 210 may request a confirmation of the automatic change from the user of the CGPG GUI 220. In some examples, the CGPG engine 210 may utilize various libraries (e.g., as shown in FIG. 12) of the resource managers in order to perform the validation.

The CGPG engine 210 may be configured to validate the generated protocol 225 in a variety of ways. For example, validation may include an evaluation of closed loop control instructions. The CGPG engine 210 may check how many times a closed loop control loop would run according to the sequencing links and protocol blocks of the encoded clinical guidance protocol 225. If the number of repetitions of a loop may cause a physiological parameter to become clinical invalid (e.g., the parameter may exceed or drop below a physically possible and/or clinically recommended value), then the CGPG engine 210 may generate a validation error message. The user needs to correct this error, for example, by adding sequencing links and protocol blocks that cause an exit from a control loop before the physiological parameter becomes clinically invalid. For example, a workflow section may direct the clinical guidance engine 235 to adjust a ventilator setting for respiratory rate by a fixed increment, wait 10 minutes, check the EtCO2 value, and repeat this loop if the EtCO2 value is not within an acceptable range. However, if this loop repeats too many times, the EtCO2 value may be driven to a clinically undesirable value. Therefore, the loop would require an addition of a counter or other check so that the loop ceases to repeat prior to reaching the clinically undesirable value.

As another example, for validation, the CGPG engine 210 may confirm that a user specification for a parameter is in a proper format (e.g., numeric format for a numeric parameter rather than a text string). This validation may also flag parameter specifications that are impossible such as a condition that a patient's age be less than zero. As a further example, the CGPG engine may confirm that a user specification for a particular protocol block is actionable and physiologically valid. For example, a protocol block condition that SpO2 exceed 100% is not physiologically valid. Therefore, this condition would always fail during protocol implementation. As another example, a protocol block may request a medical device setting or mode that is unavailable. In an implementation, the resource manager 240 may include the physiological indications library 1240 and/or the medical device library 1250 (as shown in FIG. 12) and the CGPG engine 210 may reference these libraries during validation. For example, the CGPG engine 210 may validate the generated protocol 225 based on validation information retrieved from one or more of an instructional library, a medication library, a physiological indications library, a medical device library, a protocol library, or a user account information library. The validation information may be information that the CGPG engine 210 retrieves from the respective library to use as a comparison with user specifications or sequences in the protocol. If the retrieved information contradicts or otherwise conflicts with the protocol, then the CGPG engine 210 validation process may generate an error message or a corrective action. For example, if the user of the CGPG GUI 220 provides a drug dosage, a sequence of medical steps, medical equipment instructions, user account information, etc. that in a protocol and this information conflicts with the library information, then the CGPG engine 210 validation process may generate the error message.

In an implementation, the CGPG engine 210 may invoke the resource manager to confirm that the equipment provided for in the closed-loop control is available to the user of the protocol (e.g., based on account and/or agency identification information). The validation may include a check that the demographics, such as age, gender, and/or the medical history as evidenced by previous workflow steps allow for use of particular equipment. The validation may further evaluate geographical guidelines, such as legal guidelines, regarding the use of equipment and/or procedures (e.g., based on account and/or agency identification information). In an implementation, the CGPG engine 210 may recommend that the protocol include time-based options. For example, the protocol may include a check of a time of arrival at a healthcare facility by the clinical guidance engine 235. Based on that time of arrival, the protocol may include branches for interventions of longer or shorter duration.

The validation may also include a test that a sequencing link provides for a sequence of protocol blocks where the sequence indicated is actionable and physiologically valid. For example, the CGPG engine 210 may verify that a subsequent protocol block does not indicate an action that is contradictory to a preceding protocol block. As yet another example, the CGPG engine 210 may inspect the encoded clinical guidance protocol 225 for self-consistency. Such a self-consistency validation may flag contradictory conditions and/or settings between prior and subsequent protocol blocks. For example, if a workflow is initiated based on a user entry of an age <10, a subsequent block would be flagged as invalid if that subsequent block included a condition that the age of the patient be >80.

In various examples, validation may apply to drug administration instruction blocks or medical device instruction blocks and/or protocol blocks that follow these blocks in sequence. For drug administration, the validation may confirm that the instructions for an indicated drug are available in the resource manager 240, 260, and/or 280 to support guidance at the clinical guidance UI 250. As another example, the CGPG engine 210 may compare the drug administration block to an inventory list, a drug interaction list, an age recommendation, or other drug indications (e.g., via a medication library 1230 in a resource manager) and/or may require a protocol block that checks a patient's medical record for contraindications such as allergies or incompatible current medications.

The validation may further include a check by the CGPG engine 210 that all variables in use by the generated protocol 225 are defined and that all the ports that require a connection via a sequencing link to another port are in fact connected.

In an implementation, the validation may generate outputs indicative of tests applied to one or both of the user specification or the sequencing links to ensure that the user specification and the sequence of protocol blocks are actionable and physiologically valid. For example, the CGPG engine 210 may generate a first output that indicates that the user specification is one or both of unactionable or physiologically invalid if the validation test of the user specification fails. Similarly, the CGPG engine 210 may generate a second output that indicates that one or more sequencing links are one or both of unactionable or physiologically invalid if the validation test of the sequence of protocol blocks fails.

At the stage 1080, the CGPG engine 210 may export the encoded clinical guidance protocol 225. For example, the CGPG engine 210 may export the encoded clinical guidance protocol 225 to a database configured for access by the clinical guidance engine 235. The database may be one or more of the platform resource manager 240, the local resource manager 260, or the agency resource manager 280. The clinical guidance engine 235 may access one or more encoded clinical guidance protocols 225 and implement at least a portion of the protocol(s) at the clinical guidance UI 250. Further, the clinical guidance engine 235 may implement at least a portion of the protocol(s) as background tasks without providing any controls and/or information at the clinical guidance UI 250. In an implementation, the CGPG engine 210 may receive a user selection of the export control 53e (e.g., as shown in FIG. 5A) and export the generated protocol.

Referring to FIG. 12, an example of a resource manager for a clinical guidance protocol generation and clinical guidance system is shown. A quantity of each component in FIG. 12 is an example only and other quantities of each, or any, component could be used. The system 200 in FIG. 2A may include resource managers. Specifically, the CGPG engine 210 may be communicatively coupled to a platform resource manager 240 and the clinical guidance engine 235 may be communicatively coupled to one or more of the resource managers 240, 260, and/or 280. The platform resource manager 240 may be disposed at one or more memory devices of a cloud server providing the clinical guidance protocol platform 202 (e.g., the cloud server 270). The agency resource manager may be disposed at one or more memory devices of an agency server (e.g., the agency server 295). The local resource manager 260 may be disposed at one or more memory devices at or coupled to the one or more devices that provide the clinical guidance UI 250. For example, the local resource manager 260 may be disposed at a mobile computing device (e.g., the user interface device 255) and/or a medical device 265. The platform resource manager 240, the local resource manager 260, and the agency resource manager 280 may include all of the same resources or may include some common resources and some distinct resources. In an implementation, the platform resource manager 240 may include resources associated with multiple clinical guidance user accounts associated with multiple agencies, hospitals, and other caregiving organizations. The resources may be designated according to the corresponding account. In an implementation, the agency resource manager 280 and the local resource manager 260 may include only resources associated with one or more specific accounts associated with a particular agency, hospital, or other caregiving organization. Further, the local resource manager 260 may include only resources associated with a specific caregiving individual or team within an account. In an implementation, the platform resource manager 240, the local resource manager 260, and the agency resource manager 280 may be configured to synchronize with one another periodically to align and update common resources.

The CGPG engine 210 may store information including the encoded clinical guidance protocol in the resource manager 240, 260, and/or 280. The CGPG engine 210 may store the encoded clinical guidance protocol according to account information associated with the protocol. In this manner, an agency, hospital, or other caregiving organization may access protocols associated with their account.

The resource manager 240, 260, and/or 280 may include an instructional library 1220, a medication library 1230, a physiological indication library 1240, a medical device library 1250, a protocol library 1260, and/or an account information library 1270.

The instructional library 1220 may include an instructional image library 1222, an instructional animation library 1224, and/or an instructional text library 1226. In various examples, one or more protocol blocks may cause the clinical guidance engine 235 to retrieve instructional information from the instructional library 1220 and provide the instructional information at the clinical guidance UI 250. For example, a UI instruction protocol block 860a may provide instructions as shown in FIG. 8E-2. In conjunction with those instructions, the UI instruction protocol block 860a may retrieve textual instructions, graphic instructions, and/or instructional animations from the instructional library 1220 in order to demonstrate in real-time to the user of the clinical guidance UI 250 proper procedures for implementing provided instructions. In an implementation, this protocol block may cause the clinical guidance engine 235 to retrieve medical device information from the medical device library 1250 that indicates which specific medical devices are or could be communicatively coupled to the clinical guidance engine 235. The clinical guidance engine 235 may then retrieve instructional information associated with the one or more medical devices that are or could be communicatively coupled so as to tailor the guidance to the specific needs of a user.

The medication library 1230 may include at least one of a medication indication library 1232 and a medication dosage library 1234. In an example, the CGPG engine 210 may be configured to retrieve medication specification guidelines from the medication library and provide the medication specification guidelines at the CGPG GUI 220. This may enable a user of the CGPG GUI 220 to modify the guidelines and/or select a protocol progression sequence based on the guidelines. Additionally, during protocol generation, the CGPG engine 210 may validate a user specification of a medication based on information in the medication library. Additionally, or alternatively, CGPG engine 210 may provide medication information at the CGPG GUI 220. The user of the CGPG GUI 220 may select a protocol progression sequence-based medication information provided at the CGPG GUI 220. The CGPG engine 210 may retrieve specification guidelines for medications relevant to a protocol from the medication library 1230 and provide the specification guidelines for physiological parameters at the CGPG GUI 220. For example, the CGPG engine 210 may provide the medical library information in a drop-down menu at the CGPG GUI 220.

The physiological indication library 1240 may include one or more of a physiological parameter library 1242, a patient symptom and condition library 1244, or a threshold and ranges library 1246. In an example, the CGPG engine 210 may be configured to retrieve physiological parameter information from the physiological indication library 1240 and provide the physiological parameter information at the CGPG GUI 220. This may enable a user of the CGPG GUI 220 to modify the guidelines and/or select a protocol progression sequence based on the guidelines. During protocol generation, the CGPG engine 210 may validate a user specification of a physiological parameter based on information in the physiological indication library 1240. Additionally, or alternatively, CGPG engine 210 may provide physiological parameter information at the CGPG GUI 220. The user of the CGPG GUI 220 may select a protocol progression sequence based physiological parameter information provided at the CGPG GUI. The CGPG engine 210 may retrieve specification guidelines for physiological parameters relevant to a protocol from the physiological indication library 1240 and provide the specification guidelines for physiological parameters at the CGPG GUI 220. For example, the CGPG engine 210 may provide the physiological parameter information in a drop-down menu at the CGPG GUI 220.

Referring to FIG. 13, an example of a parameter file from the parameter library is shown. One or more of the resource managers 240, 260, 280 may store a parameter file 1300. For example, the parameter library 1240 may store this file in one or more of the physiological parameter library 1242 and/or the thresholds and ranges library 1246. The parameter file 1300 may be a distributed file accessible as an editable report by the user of the CGPG GUI 220. The user of the CGPG GUI 220 may access the parameter file 1300 to create and specify parameters and attributes and edit pre-defined parameters. The parameter file 1300 is only an example of a portion of a possible parameter file and is not limiting of the disclosure.

The parameter category 1310 and subcategory 1315 are organizational attributes at the discretion of the user of the CGPG GUI 220. For instance, the user may group the parameters according to a sensor type, medical device type, patient charting entry type, etc. As an example, all non-invasive blood pressure (NIBP) parameters may be grouped together with subcategory designations as systolic or diastolic. The parameter name 1320 is a name of the parameter as used by the CGPG engine 210 during generation of the encoded clinical guidance protocol and the export name 1330 is the encoded name exported to the clinical guidance engine 235. The alias 1340 is a clinician name for the parameter according to a vernacular recognized by the user of the CGPG GUI 220 and could reflect different spoken languages (e.g., English, French, Chinese, etc.). The parameter type 1350 indicates a format type of numeric, text, binary, or option. The read only designation 1360 indicates parameters that may only be read from a medical device and/or a database but may not be entered or edited by a user. The options 1370 designate the input options for a parameter. The lower limit 1380 and the upper limit 1390 provide valid ranges for the parameter. The unit 1395 indicates a measurement unit. The description 1399 allows a user to make optional notations as to how they are using or specifying a particular parameter. In an implementation, the CGPG engine 210 may provide default values in the parameter file 1300 and the user of the CGPG GUI 220 may edit those default values. In an implementation, the CGPG engine 210 may validate an encoded clinical guidance protocol 225 based on the parameter file 1300.

Referring again to FIG. 12, the medical device library 1250 may include a medical device drivers library 1252 and/or a verified medical devices library 1254. In an implementation, medical device interaction protocol blocks may utilize device drivers to either provide instructions to the medical devices and/or to retrieve information from the devices. Further the library 1250 may include the verified medical devices library 1254 to indicate which medical devices are authorized to interact with the clinical guidance engine 235. In some examples, medical device library 1250 may provide information that enables the clinical guidance engine 235 to initiate communications with a particular medical device based on this information and/or search for such a device in a local area network. In this manner, the clinical guidance engine 235 may tailor the guidance to medical devices actually in use and capable of communications with the engine 235. In various examples, the medical device interaction protocol blocks may cause the clinical guidance engine 235 to provide an instruction (e.g., the medical device instructions 425) to at least one medical device 265 based on information in the medical device library 1250. In an implementation, the clinical guidance engine 235 may retrieve information from the medical device library 1250 such as the make and model of a particular medical device. In an implementation, the CGPG engine 210 may provide medical device information, such as make, model, and/or operational specifications for a medical device at the CGPG GUI 220 for use during protocol generation.

The protocol library 1260 comprises clinical guidance protocols generated at and/or for use by the CGPG engine 210. The encoded clinical guidance protocols generated at the CGPG GUI 220 are user-defined protocols. In other words, these are protocols defined by the user of the CGPG GUI 220. When the encoded clinical guidance protocols are generated at the CGPG GUI 220, they may be saved in the platform resource manager 240. Further, the CGPG engine 210 may provide these protocols from the library 1260 at the CGPG GUI 220 at the workflow blocks menu 515 of the CGPG GUI 220 so that a user of the CGPG GUI 220 may select these protocols and nest them within other generated protocols. Thus, one generated protocol may invoke another generated protocol in a nested fashion. As discussed above, some foundation blocks may function as initiation blocks for a nested user-defined protocol. The protocol library 1260 may also include the built-in protocols that the CGPG engine 210 may provide at the workflow blocks menu 515 of the CGPG GUI 220.

In an implementation, a second party, such as a medical director for a particular ambulance agency or hospital may retrieve and customize an encoded clinical guidance protocol 225 from the resource manager 240, 260, and/or 280 to create the customized encoded clinical guidance protocol 325. Such customization may or may not occur during a patient encounter depending on particular circumstances of the encounter. In an implementation, a protocol customization engine (e.g., the protocol customization engine 310 shown in FIG. 3) may enable customization of at least one encoded clinical guidance protocol and save the customized encoded clinical guidance protocol in a customized protocol library of the protocol library 1260.

The clinical guidance engine 235 may implement one or more of the encoded clinical guidance protocols 225 or the customized encoded clinical guidance protocols 325. Thus, generated protocols are available to but not generated by users of the clinical guidance UI 250.

The user account information library 1272 includes user account information associated with the generated and/or customized encoded clinical guidance protocols saved in the protocol library 1260. Additionally, the user account information library may include account information associated with one or more of the instructional library, the medication library, the physiological indication library, and the medical device library. The user account information library 1272 may enable the clinical guidance engine 235 and/or the clinical guidance protocol customization engine 310 to access library resources specific to a particular user account. The user account may be associated with an agency, hospital, agency system, hospital system, and/or other caregiving organization.

Referring to FIG. 14, an example of an encoded spinal cord assessment workflow generated at the CGPG GUI is shown. For example, the CGPG engine 210 may generate the protocol 1400 at the CGPG GUI 220 based on entries to the CGPG GUI 220 from the first user 20. The illustrated encoded spinal cord assessment workflow is, however, an example only and not limiting. This workflow can be altered, e.g., by having workflow blocks added, removed, rearranged, combined, and/or performed concurrently.

The example of the spinal cord assessment workflow shown in FIG. 14 includes foundation blocks that cause the clinical guidance engine 235 to provide prompts at the clinical guidance UI 250. The prompts guide the caregiver (e.g., the second user 21) through a spinal cord assessment. Furthermore, the example of the spinal cord assessment workflow shown in FIG. 14 includes foundation blocks that cause the clinical guidance engine 235 to provide instructions at the clinical guidance UI 250. These blocks enable the caregiver to request additional guidance, for example, text, graphics, or animations that provide details on how to collar a patient, how to transfer the patient to a cot, and/or how to secure the patient on the cot. Because this guidance is available upon request, the caregiver may tailor the instructions to their own level of training. If the caregiver does not need instruction, the clinical guidance UI 250 does not provide unnecessary and possibly distracting information.

To generate the spinal cord assessment workflow 1400 shown in FIG. 14, the CGPG engine 210 is configured to receive the user selection at the CGPG GUI 220 of protocol blocks including unspecified multiple-choice protocol blocks and unspecified UI instruction blocks. For example, the first user 20 may select these protocol blocks from the protocol block selection menu 502 and the CGPG GUI 220 may display these selected blocks in the graphic protocol display area 504. The first user 20 may then select these blocks within the display area to pull up the user specification window 506. The first user 20 may then provide entries to the various fillable fields in the user specification window 506 at the CGPG GUI 220. The CGPG engine 210 may then incorporate the user specification information into the unspecified protocol blocks to convert them to specified protocol blocks. For example, for each of the multiple-choice protocol blocks 1405, 1410, 1415, 1420, and 1425, the protocol blocks are specified to provide specific text at the CGPG GUI 220. For instance, the operation of block 1405 may provide the text “high risk or questionable injury mechanism” or text with a similar meaning at the CGPG GUI 220 and the operation of block 1410 provides the text “midline spinal pain or tenderness with palpation” or text with a similar meaning at the CGPG GUI 220.

The first user 20 may then indicate sequencing links between the various protocol blocks and the CGPG engine 210 may connect the protocol blocks according to the indicated sequencing links. For example, the sequencing link 1440 connects output linkage port A 1445 with the input linkage port 1450 of the subsequent protocol block. This causes a logically true answer to the text provided by protocol block 1405 to move the workflow 1400 to protocol block 1410. The sequencing link 1455 connects output linkage port B 1460 to input linkage port 1465 of protocol block 1435. This causes a logically false answer to move the workflow 1400 to protocol block 1435. The remaining output linkage ports are connected to the remaining input linkage ports so that if the answer to all of the prompts are logically true the workflow moves to the UI instruction protocol block 1430. Thus, the CGPG engine 210 receives the user indications at the CGPG GUI 220 of the sequencing links between the protocol blocks and connects the protocol blocks according to the user-specified links. Depending on the answers to the multiple-choice prompts at the clinical guidance UI 250, the clinical guidance UI 250 may provide either the instruction specified by protocol block 1430 (instructions for spinal motion restriction) or the instruction specified by protocol block 1435 (a notification to bypass spinal motion restriction).

The first user 20 may provide user specifications and sequencing links in any order (i.e., all user specifications then all sequencing links, alternating one or more user specifications with one or more sequencing links, or all sequencing links and then all user specifications). Furthermore, during this process, the first user 20 may add or delete protocol blocks.

The CGPG engine 210 may generate the spinal cord assessment workflow 1400 with the specified protocol blocks and the user-indicated sequencing links and export the generated spinal cord assessment workflow 1400 to the clinical guidance engine 235 and/or save the spinal cord assessment workflow 1400 to the resource manager 240, 260, and/or 280. As a saved workflow, the spinal cord assessment workflow 1400 may be available to the first user 20 to select and nest within a larger envelope protocol.

Referring to FIGS. 15A-15E, an example of an encoded modified early warning score (MEWS) assessment protocol generated at the CGPG GUI is shown. For example, the CGPG engine 210 may generate the protocol 1500 at the CGPG GUI 220 based on entries to the CGPG GUI 220 from the first user 20. The illustrated encoded MEWS assessment workflow is, however, an example only and not limiting. This workflow can be altered, e.g., by having workflow blocks added, removed, rearranged, combined, and/or performed concurrently.

The MEWS assessment protocol begins in FIG. 15A and the monitor block 87. The monitor block 87 causes the clinical guidance engine to implement the protocol 1500 and calculate or update the MEWS score any time the clinical guidance engine 235 receives a changed value of NIBP (from block 1510), heart rate (from block 1520), respiration rate (from block 1530), temperature (from block 1540), and/or AVPU (from block 1550). The change is based on a comparison of a new or refreshed value of NIBP, HR, RR, temperature, or AVPU with a previously received value (which may be a null value if the clinical guidance engine 235 receives a first and new value). Because protocol 1500 is a monitoring protocol, the use of the MEWS score is not limited to an initial triage and repetition of the MEWS score evaluation does not take a caregiver away from other tasks. Instead, the clinical guidance engine 235 can watch the MEWS score over the course of treatment and notify the caregiver of changes in that score if and when such a change occurs.

The protocol 1500 moves through the steps in FIG. 15A to the “A” junction that moves to FIG. 15B. Similarly, the protocol 1500 moves through the steps in FIG. 15B to the “B” junction that moves to FIG. 15C, then through the steps of FIG. 15C to the “C” junction that moves to FIG. 15D, then through the steps of FIG. 15D to the “D” junction that moves to FIG. 15E, and finally through the steps of FIG. 15E to the end block 1595. Each of the sections of the protocol 1500 shown in each of FIGS. 15A-15E evaluates one of five parameters of the MEWS assessment. The section in FIG. 15A evaluates non-invasive blood pressure (NIBP) at protocol block 1510. The section in FIG. 15B evaluates heart rate at protocol block 1520. The section in FIG. 15C evaluates respiration rate at protocol block 1530. The section in FIG. 15D evaluates temperature at protocol block 1540. Finally, the section in FIG. 15E evaluates an AVPU score in protocol block 1550 (where AVPU is an acronym for alert, verbal, pain, unresponsive as a caregiver evaluation of a patient's consciousness).

Each of the protocol blocks 1510, 1520, 1530, and 1540 is a medical device interaction protocol block. These protocol blocks cause the clinical guidance engine 235 to retrieve a physiological measurement from a medical device, such as, a blood pressure measurement, a heart rate measurement, a respiration rate measurement, and a temperature measurement. In an implementation, a single medical device, such as a patient monitor or a defibrillator/patient monitor 482 may collect these measurement through sensors (e.g., the patient interface devices 450 shown in FIG. 4A). The clinical guidance engine 235 may be communicatively coupled to the patient monitor or the defibrillator/patient monitor and/or may be communicatively coupled to one or more of the sensors.

In the example of the protocol 1500, the medical device interaction protocol blocks 1510, 1520, 1530, and 1540 are range evaluation protocol blocks configured to evaluate a range of the physiological measurement from the medical device as a background task without generating a UI control at the clinical guidance UI 250. Based on the range evaluation, a sequencing link moves the protocol 1500 to an internal logic block that sets a value of a variable. For example, if the NIBP is between 0-70 mmHg, the sequencing link 1512 moves from the output port 1514 associated with the 0-70 mmHg range to the input port 1516 of protocol block 1518. The protocol block 1518 is, for example, a value assignment protocol block that sets the NIBP score variable (e.g., “NIBPScore”) to a value of “3.” The sequencing link 1513 then moves the 1500 protocol to the junction “A” and the next section of the protocol 1500 shown in FIG. 15B.

The protocol sections in FIGS. 15B, 15C, and 15D function in a similar manner to the section illustrated in FIG. 15A. The protocol block 1520 evaluates the range of a measured heart rate and then based on that range, one of the protocol blocks 1522, 1524 or 1526 sets a value of the heart rate score variable (e.g., “HRScore”). The protocol block 1530 evaluates the range of a measured respiration rate and then based on that range, one of the protocol blocks 1532, 1534,1536, or 1536 sets a value of the respiratory rate score variable (e.g., “RRScore”). The protocol block 1540 evaluates the range of a measured temperature and then based on that range, one of the protocol blocks 1542 or 1544 sets a value of the temperature score variable (e.g., “TempScore”).

Notably, each of the protocol blocks 1510, 1520, 1530, and 1540 are the same type of protocol block (e.g., a range evaluation protocol block) however the number of output ports differs based on the user specification of these blocks that define the number of ranges to be evaluated for each block. Thus, based on the user specification, the specified protocol block may have a different number of output ports than the corresponding unspecified protocol block with the same operator.

Another feature of each of the protocol blocks 1510, 1520, 1530, and 1540 is that each of these includes an indeterminate value output port. These protocol blocks are configured to default to the indeterminate value output linkage port if a respective MEWS assessment physiological parameter is unavailable. The sequencing links connect the indeterminate value output linkage port to an input linkage port of a caregiver interactive protocol block. The caregiver interactive protocol block is configured to cause the clinical guidance engine 235 to generate a UI control at the clinical guidance UI 250. For example, the UI control may be a user prompt at the clinical guidance UI 250 for a caregiver entry of the respective MEWS assessment physiological parameter. The protocol blocks 1562, 1564, 1566, and 1568 are examples of caregiver interactive protocol blocks. The protocol blocks 1562, 1564, 1566, and 1568 cause the clinical guidance engine 235 to prompt for entry of NIBP, heart rate, respiratory rate, and temperature, respectively.

In an implementation, the clinical guidance engine 235 may define one or more intrinsic or default operations where a measurement is required to implement a protocol step, but the measurement is both unavailable and the indeterminate value output linkage port is unconnected. For example, the default operations may include an automatic prompt for the second user 21 to set up and activate a sensor, request entry of a value at the clinical guidance UI 250, contact a supervisor or request other assistance, and/or inform the second user 21 that particular information and/or equipment is necessary to proceed with guidance.

Additionally, each of these blocks includes a guidance port that causes the clinical guidance engine 235 to generate a more guidance control at the clinical guidance UI 250. If the guidance port is not connected to another block via sequencing link, the clinical guidance engine 235 may either not provide an option for guidance at the clinical guidance UI 250 or may provide an option that, if selected, causes the clinical guidance engine 235 to retrieve and display non-interactive instructions. For example, the non-interactive instructions may be a displayed document in, for example, a portable document format (PDF) or joint photographic experts group (JPEG) format. In some implementations, the displayed document may be protocol instructions from an EMS agency and/or page(s) from an instruction for use document for a medical device. However, in the non-interactive case, the clinical guidance UI 250 merely displays the information and does not generate any controls for user selection of display options (e.g., a request for further guidance, an entry confirmation control, an animation of device operation, etc.) other than, for example, to close the display window. The caregiver (e.g., the second user 21 of the clinical guidance UI 250) may select the more guidance UI control to receive additional guidance on how to procure each of the measurements that may include instructions in the form of text, animations, or graphics on how to utilize the particular medical device and/or sensor capable of providing the requested measurement.

The protocol block 1550 is a caregiver interactive block that causes the clinical guidance engine to generate a UI control at the clinical guidance UI 250. This control prompts the caregiver to select options, for example, multiple-choice options, that are linked to different output ports. The clinical guidance engine 235 may implement the multiple-choice block 1550 by displaying the four options of the AVPU score, namely fully awake (e.g., “A”), verbal (e.g., “V”), reporting or exhibiting pain (e.g., “P”) and unresponsive (e.g., “U”). Each of these options define a different assessment of a patient's level of consciousness. The least concerning of these options is fully awake where the patient is responsive to voice and has bodily motor function. The most concerning of these options is unresponsive where the victim is unconscious and shows no eye, motor, or verbal response to audible or painful stimuli. The verbal and exhibiting pain states are intermediate states. In the example of FIG. 15E, the caregiver selects one of “A,” “V,” “P,” and “U” on the clinical guidance UI 250 and then the generated protocol causes the clinical guidance engine 235 to link each these responses to a port and an associated sequencing link. The selection labels “A,” “V,” “P,” and “U” are examples only and other selection labels for the AVPU score are within the scope of this disclosure. The protocol block 1550 is linked to internal logic protocol blocks 1552, 1554, 1556, and 1558 that set a value of the AVPU variable (e.g., the variable “AVPUScore”).

The protocol block 1570 is an internal logic protocol block configured to evaluate variables internal to the 1500 protocol and generate a final output of a MEWS score from the protocol 1500. For example, the protocol block 1570 may be a computation block that evaluates a mathematical expression that assigns the sum of the component variables for the overall MEWS score (e.g., the variables “NIBPScore,” “TempScore,” “AVPUScore,” “HRScore,” and “RRScore”).

The generation of the protocol 1500 is substantially the same as the generation of the protocol 1400 as described above. The CGPG engine 210 is configured to receive user selections at the CGPG GUI 220 of a plurality of protocol blocks. These include first foundation protocol blocks configured to cause the clinical guidance engine to execute a background task without generating a clinical guidance UI control and second foundation protocol blocks configured to cause the clinical guidance engine to generate a UI control at the clinical guidance UI. The first user 20 may then provide user specifications for selected protocol blocks and the CGPG engine 210 may convert unspecified protocol blocks to specified protocol blocks based on the user specifications. Finally, the first user 20 may indicate sequencing links and the CGPG engine 210 connects the protocol blocks according to the sequencing links to generate the MEWS assessment workflow 1500. The CGPG engine 210 may export the generated MEWS assessment workflow 1500 to the clinical guidance engine 235 and/or save the MEWS assessment workflow 1500 to the resource manager 240, 260, and/or 280. As a saved workflow, the MEWS assessment workflow 1500 may be available to the first user 20 to select and nest within a larger envelope protocol.

The MEWS assessment workflow 1500 illustrates several advantages of the clinical guidance protocol system described herein. Assuming that an appropriate medical device is communicatively coupled to the clinical guidance engine 235, this workflow automates most of the MEWS assessment. The caregiver interaction may be limited to an entry of the AVPU assessment. This enables the caregiver to more efficiently provide patient care as the caregiver may proceed with the AVPU assessment while the clinical guidance engine 235 evaluates the other MEWS factors in the background without distracting the caregiver. Thus, once the caregiver completes and enters the AVPU assessment, the workflow 1500 is ready to provide the final assessment on a time scale of a computer processor. Further, the workflow 1500 may be embedded in a larger envelope workflow that may immediately begin providing care instructions and/or begin evaluating other patient parameters based on the output from the workflow 1500. Further, the workflow 1500 is optimized to only request caregiver interaction where the measurements are not available directly from the medical device. Additionally, the workflow 1500 enables a caregiver to request guidance according to their particular needs and not waste time with unneeded guidance or waste time struggling with a procedure where guidance would be beneficial.

MEWS is but one example of scoring workflows that may be encoded into workflows using the CGPG engine 210. Other examples of medical scores and assessments include, but are not limited to, Glasgow coma scale (GCS), quick sequential organ failure assessment (qSOFA), vision-aphasia-neglect screening (VAN), national early warning score (NEWS), targeted real-time early warning system (TREWS), sequential organ failure assessment (SOFA), National Institutes of Health stroke scale (NIHSS), functional assessment staging tool (FAST), recognition of stroke in the emergency room (ROSIER), amplitude spectrum area (AMSA), etc. These scores all provide checklist or calculable quantification of a patient's medical state vis-à-vis a particular medical condition or set of medical conditions. Scoring enables a triage type of evaluation to efficiently guide a caregiver towards intervention steps.

Referring to FIG. 16, an example of an encoded clinical guidance protocol that evaluates several different medical evaluation scores is shown. As illustrated in FIG. 16, workflows for GCS 1610, MEWS 1620, qSOFA 1630, NIHSS 1640, and VAN 1650 may be implemented in parallel using the multi-threaded workflow 1600. Although the workflow 1600 includes these five scores, it could include any number of scores (e.g., a number of scores across many orders of magnitude 10, 100, 1000, 10,000, etc.) that a user of the CGPG GUI 220 deems relevant for a particular treatment workflow. The workflow 1600 will return a final score for each of the scoring workflows shown. The workflow 1600 may be embedded within a larger workflow that determines an intervention path based on the relative values of the scoring workflows within 1600. The illustrated workflow in FIG. 16 is an example only and not limiting. This workflow can be altered, e.g., by having workflow blocks added, removed, rearranged, combined, and/or performed concurrently.

A human caregiver can only perform these assessments serially and, in fact, medical training specifically insists on a serial and methodical assessments so that no steps are forgotten. In contrast, an encoded clinical guidance protocol can enable the clinical guidance engine 235 to evaluate these assessments in parallel. Such parallel processing not only reduces the time to intervention but enables a discovery of cross-correlated factors that may be missed in a serial assessment. Additionally, the clinical guidance engine 235 may repeat one or more of these score assessments and/or multi-threaded assessment workflows as patient care progresses and thereby calculate and monitor scoring trends. Such dynamic evaluations enable ongoing and updated triage that may enable an improvement in care because developing, deteriorating, and/or improving conditions may be taken into account by the clinical guidance engine 235. Additionally, different scores are more sensitive to different conditions that may pose varying threats to a patient's health over time. Interventions may need to be modified over time but only a monitoring and trending of multiple scores may identify these issues. As a further consideration, a medical director may limit the number of conditions included in a score assessment when performed by a person. However, the encoded clinical guidance protocol enables the medical director to include a much larger number of factors without concerns for delayed care or caregiver confusion. Thus, for example rather than limiting a MEWS score to blood pressure, heart rate, temperature, AVPU, and respiratory rate, the MEWS score may also include urine output, FiO2, body mass index, age, etc. Further, the granularity of binning for the various factors may be increased in order to more narrowly and accurately categorize a patient's state.

Referring to FIGS. 17A-17B, an example of an encoded acute respiratory failure (ARF) protocol generated at the CGPG GUI is shown. The protocol 1700 has two sections, a first section in FIG. 17A that proceeds to the “E” junction and then to the remainder of the workflow shown in FIG. 17B. In practice, the ARF protocol 1700 could be embedded in a larger envelope protocol to handle instances of acute respiratory failure. The illustrated encoded ARF workflow is, however, an example only and not limiting. This workflow can be altered, e.g., by having workflow blocks added, removed, rearranged, combined, and/or performed concurrently.

The protocol blocks in the ARF protocol 1700 include first foundation protocol blocks configured to cause the clinical guidance engine 235 to execute a background task without generating a clinical guidance UI control and second foundation protocol blocks configured to cause the clinical guidance engine 235 to generate a UI control at the clinical guidance UI 250. The first foundation protocol blocks include medical device interaction protocol blocks such as the protocol blocks 1750 and 1770 shown in FIG. 17B. These blocks are configured to cause the clinical guidance engine 235 to retrieve respiratory parameters measured by a communicatively coupled medical device (e.g., a patient monitor and/or defibrillator/patient monitor 482 coupled to SpO2 and CO2 sensors 454 and 456 as shown for example in FIG. 4A). The first foundation protocol blocks may also include internal logic protocol blocks such as the protocol blocks 1760 and 1780 shown in FIG. 17B. For example, the protocol block 1760 may be a value assignment protocol block that sets a variable value for the peak inspiratory pressure (PIP) ventilation parameter. The value of the PIP variable (e.g., “PIP”) is increased by two (e.g., “+2”) if the output from the preceding block 1770 indicates that CO2 and SpO2 are within particular values.

The section of the protocol 1700 shown in FIG. 17B forms a monitoring loop 1790. This monitoring loop evaluates CO2 and SpO2 measurements in response to a mechanical ventilation intervention provided based on the built-in bi-level ventilation workflow block 1730. The ARF protocol 1700 provides caregiver interactive blocks 1710, 1720, and 1740. The protocol blocks 1710 and 1720 enable UI instructions to the caregiver to initiate hospital transport and provide a medication, e.g., a bronchodilator. For example, the protocol block 1710 may be a UI instruction protocol block and the protocol block 1720 may be a medication administration protocol block.

The protocol block 1740 enables a UI notice that once bi-level ventilation is underway, the caregiver and the system are in a monitoring mode. However, after the block 1740, the caregiver may proceed with other care activities while the clinical guidance engine 235 under instruction from the protocol 1700 monitors the patient in the background. Thus, the workflow reduces caregiver distraction and offloads caregiver tasks, such as setting a timer to remind the caregiver to manually recheck measurements, to the clinical guidance engine 235. The protocol blocks 1750 and 1770 repeat checks on the CO2 and SpO2 measurements while the protocol block 1760 defines and introduces a delay time in the ARF workflow 1700 to account for delayed patient responses to ventilation interventions. For example, the protocol block 1760 may be the delay protocol block. In this manner, the workflow 1700 is able to recheck the patient every 600 seconds and adjust the inspiratory positive airway pressure (IPAP) as needed based on these rechecks. The block 1780 causes the clinical guidance engine 235 to send a medical device instruction 425 to raise IPAP by 2 cm H₂0.

The generation of the protocol 1700 substantially the same as the generation of the protocols 1400 and 1500 as described above. The CGPG engine 210 is configured to receive user selections at the CGPG GUI 220 of a plurality of protocol blocks. These include first foundation protocol blocks configured to cause the clinical guidance engine to execute a background task without generating a clinical guidance UI control and second foundation protocol blocks configured to cause the clinical guidance engine to generate a UI control at the clinical guidance UI. The protocol blocks further include a built-in workflow block, e.g., the bi-level ventilation workflow block. The first user 20 may then provide user specifications for selected protocol blocks and the CGPG engine 210 may convert unspecified protocol blocks to specified protocol blocks based on the user specifications. Finally, the first user 20 may indicate sequencing links and the CGPG engine 210 connects the protocol blocks according to the sequencing links to generate the ARF workflow 1700. The CGPG engine 210 may export the generated ARF workflow 1700 to the clinical guidance engine 235 and/or save the ARF workflow 1700 to the resource manager 240, 260, and/or 280. As a saved workflow, the ARF workflow 1700 may be available to the first user 20 to select and nest within a larger envelope protocol.

Referring to FIGS. 18A-18B an example of an encoded CPR advisory protocol generated at the CGPG GUI is shown. The encoded CPR advisory protocol 1800 is an example only and not limiting of the disclosure. This workflow can be altered, e.g., by having workflow blocks added, removed, rearranged, combined, and/or performed concurrently. The CPR advisory protocol 1800 illustrated in FIGS. 18A and 18B is an example of a guideline enabled AMSA CPR advisory protocol. As described below, an encoded CPR advisory protocol could be guideline driven without AMSA considerations or be a strictly AMSA driven protocol.

One common way to treat ventricular fibrillation (VF) is through the use of an electrical defibrillator that delivers a relatively high voltage shock to the heart in order to force it back to a normal, consistent, and strong rhythm. People undergoing ventricular fibrillation may be more receptive to a defibrillating shock in some instances compared to others. For example, research has determined that a computation of amplitude spectrum area (AMSA), or other computational methods that use either time-based or spectrum-based analytic methods on electrocardiogram (ECG) data to calculate a prediction of defibrillation shock success, may indicate whether a shock that is delivered to a person will likely result in successful defibrillation or will instead likely fail. Often the calculation of AMSA involves complex computation like a Fast Fourier Transform, which is not a calculation that is possible in real-time during the treatment of a patient without a computer processor.

Evaluating AMSA during clinical guidance for chest compressions, ventilations, and defibrillation in response to cardiac arrest enables delivery of a shock only when the likelihood exceeds some clinically determined threshold value at which the benefit of likely defibrillation outweigh the dis-benefits of harming the patient. Trends in the AMSA values may also indicate the state of a victim over the course of a VF episode. The general phases of cardiac arrest or VF may be identified, in one representation, as three separate phases (though there may be some overlap at the edges of the phases): electrical, circulatory, and metabolic. The electrical phase is the first several minutes of an event and marks a period during which electric shock can be particularly effective in defibrillating the victim's heart and returning the victim to a relative satisfactory condition. The circulatory phase appears to mark a decrease in effectiveness for electric shock in defibrillating the victim, and particularly in the absence of chest compressions performed on the victim. As a result, the appropriate clinical guidance may be to stop advising shocks during such a phase (or to advise a shock only when other determinations indicate that a shock would be particularly likely to be effective) and instead to advise forceful CPR chest compressions. Such forceful compressions may maximize blood flow through the heart tissue and other parts of the body so as to extend the time that the victim may survive without lasting or substantial damage. In the metabolic phase, chest compressions may be relatively ineffective as compared to the circulatory phase. For example, where tissue has become ischemic, such as in circulatory phase, the tissue may react favorably to the circulation of blood containing some oxygen, but where tissue has become severely ischemic, such as in the metabolic phase, the introduction of too much oxygen may be harmful to the tissue. As a result, more gentle compressions for the first period, such as 30 seconds, may need to be advised in the metabolic phase before compressions are provided with full force. Other treatments that may be useful in the metabolic phase include extracorporeal circulation and cooling, either alone, in combination with each other, or in combination with other pharmacological treatments. In any event, observation of elapsed time since an event has begun and/or observation of the phase in which a victim is in, may be used to control a device or instruct a rescuer to switch from a first mode of providing care to a second mode of providing care in which the parameters of the provided care differ (e.g., speed or depth of chest compressions may change, temperature-based therapy may be provided or stopped, or pharmaceuticals may be administered).

In general, evaluation of AMSA values and AMSA value trends during treatment of cardiac arrest enables a caregiver to tailor the intervention to the particular patient state rather than applying a global standard of care for the intervention that cannot take into account the patient state. However, relying on individual caregivers to continuously evaluate AMSA and determine a course of treatment for a particular patient is risky because of variations in caregiver training and the difficulty of making complex medical treatment decisions within the five to ten minute window available to prevent irreversible damage from a cardiac arrest. With an encoded clinical guidance protocol such as the protocol 1800 illustrated in FIGS. 18A and 18B, the clinical guidance engine 235 can provide this guidance. With such an encoded protocol, a standard of care is enforced based on the protocol, but this standard of care is tailored to the specific patient by taking the AMSA values into account in the decision making of the protocol. The encoded clinical guidance protocol 1800 enables the clinical guidance engine 235 to handle all of the AMSA calculations and evaluations of values and trends in the background and then provides the caregiver with simple instructions based on these evaluations.

At the block 1803, the encoded protocol 1800 sets an initial AMSA value at the start of chest compressions for cardiopulmonary resuscitation (CPR), e.g., as instructed at block 1806. Block 1809 initializes a compression counter and block 1812 sets a timer. While the timer is running, block 1815 evaluates return of spontaneous circulation (ROSC). For example, the clinical guidance engine 235 may identify ROSC based on sensor signals from a patient monitor/defibrillator. If the clinical guidance engine 235 identifies ROSC, then block 1818 provides an instruction to stop the compressions and ventilations. Compressions past a point of ROSC can cause harm in the form of re-arrest and once ROSC occurs the clinical guidance engine 235 can exit the protocol 1800 at the end block 1821. The clinical guidance engine 235 can guide the caregiver in interventions other than CPR compressions and ventilations.

One advantage of the protocol 1800 is that the protocol can provide guidance to the caregiver according to medical director standards encoded in the protocol 1800 that account for ROSC without having to identify a specific number of compressions to administer and/or a fixed time interval in between checks for ROSC. All caregivers using the protocol 1800 on any patient follow the same guidance but that guidance is tailored to the specific patient conditions.

Continuing with the protocol 1800, the block 1824 watches for 30 compressions and then checks ROSC at 1815 and either instructs to stop compressions or provides a ventilation instruction at block 1833. The protocol also provides for a 1 second delay at block 1827 before the ROSC check and updates a total compression count at the block 1824. In an implementation, the protocol 1800 enables the clinical guidance engine 235 to receive signals indicative of compressions from a medical device and/or a sensor. For example, the clinical guidance engine 235 may receive signals indicative of compressions from a patient monitor/defibrillator 482 coupled to a compression sensor 451 and/or the clinical guidance engine 235 may be communicatively coupled to the compression sensor 451.

Following the instruction to provide ventilations at block 1833, the protocol moves to FIG. 18B and block 1836 for an evaluation of the AMSA value. According to block 1836, if the AMSA value is greater than or equal to 15, the protocol evaluates the shockable rhythm assessment (i.e., the ECG assessment) from the patient monitor/defibrillator at block 1839. Block 1842 advises administration of a defibrillation shock based both on the AMSA value and the rhythm analysis. Block 1842 also enables the clinical guidance engine 235 to provide an option for guidance at the clinical guidance UI 250. Block 1845 reevaluates ROSC. Blocks 1848 and 1851 update an epinephrine counter. The epinephrine counter counts compression cycles in order to provide an instruction to administer epinephrine after a certain number (e.g., 10) of chest compression cycles. The epinephrine counter is reset to zero at block 1848 when a shock is administered. If the clinical guidance engine 235 detects ROSC at block 1845, the protocol returns to the instruction to stop CPR at block 1818. The protocol then either provides an epinephrine dose instruction at block 1854 or returns to block 1806 for further chest compression instructions at block 1806. The administration of epinephrine may be confirmed by a user input to the clinical guidance UI 250 confirming administration of an epinephrine dose and/or by a signal from a drug administration device configured to communicatively couple to the clinical guidance engine 235.

Another advantage of the protocol 1800 is that the clinical guidance engine 235 tracks the epinephrine administration interval. This relieves the caregiver from having to count compression cycles or use another time keeping mechanism to provide proper dosing intervals in the midst of the repetitive provision of compressions, ventilations, and shocks. In the case of manual chest compressions, the chest compressions are physically exhausting which further strains a caregiver's ability to track repetitive procedures.

Returning to block 1836, if the AMSA value is less than 15, rather than proceeding to a shockable rhythm analysis at block 1839, the protocol 1800 evaluates a fractional change in the AMSA value at block 1860. Based on this change, the protocol either proceeds to the shockable rhythm analysis at block 1839 or checks the trend of the changes in AMSA at block 1863. Based on the trend, the protocol again either returns to a rhythm analysis at block 1839 or updates compression and epinephrine counters at blocks 1866 and 1869, respectively. The CGPG engine 210 enables the user of the CGPG GUI 220 to specify the conditional evaluation metrics for each of the conditional evaluation blocks 1836, 1860, and 1863. Further, the CGPG engine 210 enables the user of the CGPG GUI 220 to specify the condition evaluation tools through the ability to include and select amongst one or more of these conditional evaluation blocks. The AMSA evaluations in blocks 1836, 1860, and 1863 are examples only and other evaluations, numerical metrics, and evaluation combinations and sequences are within the scope of the disclosure.

Block 1866 updates a compression cycle counter for each set of 30 compressions followed by ventilations. Once this compression cycle counter reaches four, the protocol moves to block 1839 and 1842 for rhythm analysis and shock when advised by the rhythm analysis. Block 1869 updates an epinephrine counter. This counter counts compression cycles so that an epinephrine dose is advised after 10 compression cycles in the absence of a delivered shock. The epinephrine counter is reset to zero at block 1851 after every shock. Note that blocks 1848 and 1851 reset the epinephrine counter when it reaches 10 but the protocol only proceeds to block 1848 if the shockable rhythm is found at block 1839 and a shock is delivered at block 1842. Note further that block 1842 advises a shock. In an implementation, the clinical guidance engine 235 may provide user guidance at the clinical guidance UI 250 and/or at the patient monitor/defibrillator 482 that a shock is advised, and the caregiver may then administer a shock by pressing a shock button on the patient monitor/defibrillator 482. In an implementation, the clinical guidance engine 235 may provide a shock instruction to the patient monitor/defibrillator 482 through a communicative coupling. In response to the shock instruction, the patient monitor/defibrillator 482 may provide a caregiver instruction or may automatically deliver the shock. At the block 1872, the protocol 1800 proceeds to ECG analysis and shock delivery if the number of compression cycles is less than four (e.g., via the sequencing link 1899). If the number of compression cycles is four, then the protocol 1800 proceeds back to block 1806 shown in FIG. 18A.

Standard guidelines for CPR, for example, as promulgated by resuscitation science advisory organizations (e.g., the American Heart Association, the Red Cross, the International Liaison Committee on Resuscitation, etc.) provide specific cardiac arrest care guidelines regarding factors such as a quantity of chest compressions in a single compression cycle, a quantity of compression cycles between defibrillation shock administration, quantity and frequency of ventilations, quantity and frequency of epinephrine administrations, pause times during compressions, etc. These guidelines are adjusted over time to reflect advances and changes in cardiac arrest science. The protocol 1800 incorporates guidelines and supplements these guidelines with the AMSA analysis. The guidelines are reflected (a) in blocks 1815, 1824 and 1833, which provide for ventilations in the absence of ROSC after every compression cycle of 30 chest compressions, (b) in blocks 1872, 1875, and 1839, which provide for an administration of shock in the presence of a shockable heart rhythm after four compression cycles with intervening ventilations (or approximately every two minutes), and (c) in blocks 1848, 1851, 1845, and 1869 which provide for an administration of epinephrine after ten compression cycles with intervening ventilations following a shock delivery. In an example, a CPR advisory protocol based strictly on guidelines without AMSA supplementation would exclude blocks 1803, 1836, 1860, and 1863. Such a protocol would include the other blocks and sequencing links shown in FIGS. 18A and 18B, or similar blocks and sequencing links according to the CPR guidelines. The presence of the AMSA blocks 1803, 1836, 1860, and 1863 enable the protocol 1800 to provide guidance for the administration of shocks sooner than the guidelines (i.e., sooner than an approximately two-minute interval at the completion of four compression cycles with intervening ventilations). This allows the protocol 1800 to advise for more frequent shocks based on the relative responsiveness of the patient's heart to defibrillation shock as determined by AMSA. In an example, the CPR advisory protocol could be strictly AMSA driven, in which case the guideline default of a shock at least every two minutes would not apply. Such a protocol would only reach block 1839 from blocks similar to 1839, 1860, and 1863 but would not reach block 1839 based on a compression cycle count such as that in blocks 1872 and 1875. In other words, a strictly AMSA driven CPR advisory protocol would exclude at least sequencing link 1899.

Referring to FIGS. 19A-19C, an example of an encoded trend monitoring protocol generated at the CGPG GUI is shown. The encoded trend monitoring protocol 1900 is an example only and not limiting of the disclosure. This workflow can be altered, e.g., by having workflow blocks added, removed, rearranged, combined, and/or performed concurrently. The protocol 1900 provides examples of trend monitoring in the context of monitoring EtCO2 trends. Embedding this protocol within a larger protocol enables the larger protocol to receive outputs from the protocol 1900 that indicate whether the EtCO2 level is high, normal, or low and whether the EtCO2 level is improving, deteriorating, or stable. With these outputs, the larger protocol can provide instructions to a caregiver or medical device and/or can evaluate another condition and/or set of physiological parameters in light of the EtCO2 status output by protocol 1900. For example, the protocol block 1905 evaluates the EtCO2 value and sets an EtCO2 status to “high” in block 1910 if the EtCO2 value exceeds 50 mmHg. Block 1915 evaluates the EtCO2 level relative to 20 mmHg. If the EtCO2 value is above 20 mmHg and below 50 mmHg, then block 1925 sets the EtCO2 status to “normal.” If the EtCO2 value is below 20 mmHg, the block 1920 sets the EtCO2 status to “low.” Following the status evaluation of high, low, or normal, the protocol 1900 evaluates whether the EtCO2 is improving, deteriorating, or stable. Blocks 1930, 1940, and 1965 evaluate whether the difference between the EtCO2 value measured at a first time and the EtCO2 value measured at a second time is positive and greater than five. Blocks 1935, 1955, and 1965 evaluate whether the difference between the EtCO2 value measured at a first time and the EtCO2 value measured at a second time is negative and less than five. If the difference is positive and greater than five, then block 1950 sets the trend status to “improving.” If the difference is negative and less than five, then block 1945 sets the trend status to deteriorating. If the difference is between negative five and positive five, then block 1960 sets the trend status to stable. A similar protocol may be applied to physiological parameters other than EtCO2 to similarly determine a determination if the physiological parameter is high, low, or normal and to determine if the physiological parameter is deteriorating, improving, or stable. The user of the CGPG GUI 220 may determine the particular tests to apply to each physiological parameter in order to categorize the status and trend of the physiological parameter.

While certain embodiments have been described, these embodiments have been presented by way of example only and are not intended to limit the scope of the present disclosures. Indeed, the novel methods, apparatuses and systems described herein can be embodied in a variety of other forms; furthermore, various omissions, substitutions and changes in the form of the methods, apparatuses and systems described herein can be made without departing from the spirit of the present disclosures. The accompanying claims and their equivalents are intended to cover such forms or modifications as would fall within the scope and spirit of the present disclosures.

1. A system for generating encoded clinical guidance protocols, the system comprising:
a clinical guidance protocol generation (CGPG) graphical user interface (GUI), and
a clinical guidance protocol platform comprising a CGPG engine configured to:
receive a user selection at the CGPG GUI of a plurality of protocol blocks comprising at least one unspecified protocol block;
receive a user specification at the CGPG GUI for the at least one unspecified protocol block,
convert the at least one unspecified protocol block to at least one specified protocol block based on the user specification,
receive user indications at the CGPG GUI of sequencing links between the plurality of protocol blocks,
connect the plurality of protocol blocks according to the user-indicated sequencing links,
generate an encoded clinical guidance protocol comprising the plurality of protocol blocks connected by the user-indicated sequencing links, and
export the encoded clinical guidance protocol to a clinical guidance engine configured to implement at least a portion of the encoded clinical guidance protocol at a clinical guidance user interface (UI).

2. The system of claim 1, wherein:
the at least one unspecified protocol block comprises at least one unspecified foundation block,
the plurality of protocol blocks comprises at least one workflow block, and
the CGPG engine is configured to:
convert the at least one unspecified foundation block to at least one specified foundation block based on the user specification, and
generate the encoded clinical guidance protocol comprising the at least one specified foundation block and the at least one workflow block connected by the sequencing links.

3. (canceled)

4. The system of claim 2, wherein the at least one workflow block comprises a built-in workflow or an imported workflow, the built-in workflow comprising one of:
a bi-level ventilation workflow, a continuous positive airway pressure (CPAP) ventilation workflow, a spirometry workflow, a non-rebreather (NRC) mask supplemental oxygen workflow, or a nasal cannula supplemental oxygen workflow; and
the imported workflow comprising an encoded clinical guidance protocol previously generated at the CGPG GUI.

5. (canceled)

6. (canceled)

7. The system of claim 4, wherein the encoded clinical guidance protocol previously generated at the CGPG GUI comprises one of a spinal cord immobilization workflow, an acute respiratory failure (ARF) workflow, a modified early warning score (MEWS) workflow, a multi-threaded score evaluation workflow, a CPR advisory workflow, or a trend monitoring protocol.

8. (canceled)

9. The system of claim 1, wherein
the CGPG engine is configured to incorporate the user specification into the at least one unspecified protocol block to convert the at least one unspecified protocol block to the at least one specified protocol block, and
the at least one specified protocol block is configured to cause the clinical guidance engine to implement the encoded clinical guidance protocol according to the incorporated user specification.

10. The system of claim 9, wherein
the at least one unspecified protocol block comprises: (a) an operator and (b) an undefined operation, and the at least one specified protocol block comprises (a) the operator and (b) a defined operation based on the user specification.

11. The system of claim 10, wherein
the operator is configured to cause the clinical guidance engine to execute the defined operation as a background task without generating a clinical guidance UI control, the operator comprising one of an evaluation or a command for the clinical guidance engine, the evaluation comprising a conditional evaluation or a range evaluation, and the command comprising a refresh command or a timer command.

12. (canceled)

13. (canceled)

14. (canceled)

15. (canceled)

16. The system of claim 10, wherein the operator comprises a guidance operator and the unspecified protocol block comprises at least one instruction operator, at least one reference operator, or a series of operators comprising the at least one instruction operator and the at least one reference operator, and
the operator is configured to cause the clinical guidance engine to generate a clinical guidance UI control to execute the defined operation.

17. (canceled)

18. The system of claim 16, wherein the operator is configured to cause the clinical guidance engine to generate one of:
(a) a delay clinical guidance UI control,
(b) a user input clinical guidance UI control,
(c) a drug administration clinical guidance UI control,
(d) a caregiver instruction clinical guidance UI control,
(e) a multiple-choice clinical guidance UI control,
(f) an alert clinical guidance UI control,
(g) a guidance request control, or
(h) a checklist clinical guidance UI control.

19. (canceled)

20. The system of claim 1, wherein the clinical guidance UI is configured to implement at least the portion of the encoded clinical guidance protocol with medical device information for at least one medical device communicatively coupled to a device providing the clinical guidance UI.

21. The system of claim 1, wherein the CGPG engine is configured to: receive a user entry of an encoded clinical guidance protocol attribute comprising one or more of a protocol name, a protocol input comprising an input to the encoded clinical guidance protocol from the clinical guidance engine, a protocol output an output from the encoded clinical guidance protocol to the clinical guidance engine, or a protocol variable.

22. (canceled)

23. (canceled)

24. (canceled)

25. The system of claim 1, wherein
each of the plurality of protocol blocks comprises (a) an entry linkage port and (b) one or more output linkage ports, and
the user indications of the sequencing links comprises user selections of:
at least one output linkage port of a first protocol block of the plurality of protocol blocks, and
one entry linkage port of at least one second protocol block of the plurality of protocol blocks.

26. (canceled)

27. The system of claim 25, wherein:
each of the one or more output linkage ports is associated with a sequencing attribute that indicates a sequencing relationship between the first protocol block and the at least one second protocol block.

28. The system of claim 25, wherein:
at least one protocol block of the plurality of protocol blocks is a user input protocol block configured to generate a user input clinical guidance UI control, and
the one or more output linkage ports comprise an entry confirmation port and an entry cancelation port.

29. The system of claim 25, wherein the one or more output linkage ports comprise an advance port, wherein a protocol block having the advance port comprises one of:
(a) a wait time protocol block configured to cause the clinical guidance engine to generate a wait time clinical guidance UI control,
(b) a drug administration protocol block configured to cause the clinical guidance engine to generate a drug administration clinical guidance UI control,
(c) an alert protocol block configured to cause the clinical guidance engine to generate an alert clinical guidance UI control,
(d) a checklist protocol block configured to cause the clinical guidance engine to generate a checklist clinical guidance UI control, or
(e) an instruction block comprising a guidance output port and configured to cause the clinical guidance engine to generate a request guidance UI control.

30. (canceled)

31. The system of claim 25, wherein:
at least one protocol block of the plurality of protocol blocks comprises at least one of:
a UI instruction protocol block configured to generate a caregiver instruction clinical guidance UI control,
a multiple-choice protocol block configured to generate a multiple-choice clinical guidance UI control,
a protocol block configured to configured to cause the clinical guidance engine to execute conditional evaluation without generating a clinical guidance UI control,
a range evaluation protocol block configured to cause the clinical guidance engine to execute a range evaluation without generating a clinical guidance UI control,
a timer protocol block configured to cause the clinical guidance engine to execute a timer command without generating a clinical guidance UI control,
a refresh protocol block configured to cause the clinical guidance engine to obtain an updated parameter value from a medical device without generating a clinical guidance UI control, or
an internal logic protocol block configured to cause the clinical guidance engine to execute an internal protocol operation without generating a clinical guidance UI control.

32. (canceled)

33. (canceled)

34. (canceled)

35. (canceled)

36. (canceled)

37. (canceled)

38. The system of claim 1, wherein the CGPG engine is configured to validate the user specification and the sequencing links prior to an export of the encoded clinical guidance protocol, based on validation information retrieved from one or more libraries by the CGPG engine, the one or more libraries comprising one or more of an instructional library, a medication library, a physiological indications library,
a medical device library, a protocol library, or a user account information library.

39. (canceled)

40. The system of claim 38, wherein the validation comprises at least one of:
a first test that the user specification is actionable and physiologically valid, or
a second test that the sequencing links provide for a sequence of protocol blocks that is actionable and physiologically valid, and
wherein the validation generates a corrective action if either of the first test or the second test fail.

41. The system of claim 40, wherein the corrective action comprises one or more of an automated correction with a prompt for user confirmation or a prompt for a user correction, and the first test confirms that a value for a parameter provided in the user specification conforms to a format of the parameter.

42. (canceled)

43. The system of claim 38, wherein the validation comprises at least one of:
a first test that the user specification is actionable and physiologically valid, or
a second test that the sequencing links provide for a sequence of protocol blocks that is actionable and physiologically valid, and,
respectively,
the validation generates an output indicative of the user specification being one or both of unactionable or physiologically invalid if the first test fails, and
the validation generates an output indicative of the sequencing links being one or both of unactionable or physiologically invalid if the second test fails.

44. (canceled)

45. The system of claim 38, wherein:
the user specification and the sequencing links define a closed loop control sequence for a medical device, and
the CGPG engine is configured to:
export the encoded clinical guidance protocol to the clinical guidance engine with an instruction to execute the closed loop control sequence,
receive a parameter summary from the closed loop control sequence, and
provide a corrective action based on the parameter summary.

46. (canceled)

47. (canceled)

48. (canceled)

49. The system of claim 1, wherein the CGPG GUI is configured to provide a protocol block selection menu, a graphic protocol display area, and a user specification window.

50. The system of claim 49, wherein the protocol block selection menu displays the plurality of protocol blocks available for selection.

51. The system of claim 50, wherein the CGPG engine is configured to receive the user selection of the plurality of protocol blocks via a drag and drop operations from the protocol block selection menu to the graphic protocol display area.

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. The system of claim 1, wherein at least one protocol block of the plurality of protocol blocks is configured to cause the clinical guidance engine to utilize data from at least one medical device configured to communicatively couple to the clinical guidance engine, wherein the data comprises one or more of a patient status parameter or a device status parameter.

66. (canceled)

67. The system of claim 65, wherein the user specification comprises a type of data and a type of medical device.

68. (canceled)

69. (canceled)

70. The system of claim 1, wherein the clinical guidance engine is disposed at a device comprising a display, the device comprising a mobile computing device or a medical device, and is configured to:
communicatively couple to a cloud server providing the clinical guidance protocol platform,
receive the exported encoded clinical guidance protocol, and
provide the clinical guidance UI at least in part at the display.

71. The system of claim 1, wherein the clinical guidance engine is disposed at a cloud server and is configured to:
communicatively couple to a device comprising a display, the device comprising a mobile computing device or a medical device,
host a clinical guidance application at the device, and
provide the clinical guidance UI at least in part at the display via the hosted clinical guidance application.

72. The system of claim 1, comprising at least one resource manager communicatively coupled to the CGPG engine and the clinical guidance engine, the at least one resource manager comprising a platform resource manager and at least one of a local resource manager or an agency resource manager and wherein the local resource manager and the agency resource manager are configured to synchronize with the platform resource manager.

73. (canceled)

74. (canceled)

75. The system of claim 72,
wherein the encoded clinical guidance protocol is associated with user account information, and
the clinical guidance engine is configured to store the encoded clinical guidance protocol in the at least one resource manager according to the user account information.

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. (canceled)

103. (canceled)

104. The system of claim 1 wherein the encoded clinical guidance protocol comprises a first workflow sequenced to a second workflow, wherein the first workflow generates a parameter output, and the second workflow evaluates the parameter output at one or more constituent protocol blocks.

105. The system of claim 104, wherein the parameter output comprises a trend evaluation for a physiological parameter, the trend evaluation comprising one of improving, deteriorating, or stable.

106. (canceled)

107. (canceled)

108. (canceled)

109. (canceled)

110. (canceled)

111. (canceled)

112. (canceled)

113. The system of claim 1, wherein the encoded clinical guidance protocol comprises a modified early warning score (MEWS) assessment workflow, and the CGPG engine is configured to:
receive the user selection at the CGPG GUI of the plurality of protocol blocks comprising:
(a) a plurality of first foundation protocol blocks configured to cause the clinical guidance engine to execute a background task without generating a clinical guidance UI control, and
(b) a plurality of second foundation protocol blocks configured to cause the clinical guidance engine to generate a UI control at the clinical guidance UI,
receive the user specification at the CGPG GUI for unspecified first and second foundation blocks,
convert the unspecified first and second foundation blocks to specified first and second foundation blocks based on the user specification,
receive the user indications at the CGPG GUI of the sequencing links between the specified first and second foundation blocks,
connect the specified first and second foundation blocks according to the user-indicated sequencing links,
generate the MEWS assessment workflow comprising the specified foundation protocol blocks connected by the user-indicated sequencing links, and export the MEWS assessment workflow to the clinical guidance engine configured to implement the MEWS assessment workflow at the clinical guidance UI.

114. (canceled)

115. The system of claim 113, wherein the plurality of first foundation protocol blocks comprise medical device interaction protocol blocks configured to cause the clinical guidance engine to retrieve MEWS assessment physiological parameters from a communicatively coupled medical device.

116. (canceled)

117. The system of claim 115, wherein:
each medical device interaction protocol block comprises an indeterminate value output linkage port and is configured to default to the indeterminate value output linkage port if a respective MEWS assessment physiological parameter is unavailable, and
the sequencing links connect the indeterminate value output linkage port to an input linkage port of a protocol block configured to cause the clinical guidance UI to generate user prompt at the clinical guidance UI for a caregiver entry of the respective MEWS assessment physiological parameter.

118. (canceled)

119. (canceled)

120. (canceled)

121. (canceled)

122. (canceled)

123. (canceled)

124. The system of claim 1, wherein the encoded clinical guidance protocol comprises an acute respiratory failure (ARF) workflow, the CGPG engine configured to:
receive the user selection at the CGPG GUI of the plurality of protocol blocks comprising foundation protocol blocks and a bi-level ventilation built-in workflow block,
receive the user specification at the CGPG GUI for unspecified foundation protocol blocks,
convert the unspecified foundation protocol blocks to specified foundation protocol blocks based on the user specification,
receive the user indications at the CGPG GUI of the sequencing links between the specified foundation protocol blocks and between the foundation protocol blocks and the bi-level ventilation built-in workflow block,
connect the specified foundation protocol blocks and the bi-level ventilation built-in workflow block according to the user-indicated sequencing links,
generate the ARF workflow comprising the specified foundation protocol blocks and the bi-level ventilation built-in workflow block connected by the user-indicated sequencing links, and
export the ARF workflow to the clinical guidance engine configured to implement the ARF workflow at the clinical guidance UI.

125. (canceled)

126. The system of claim 124, wherein the foundation protocol blocks comprise:
(a) a plurality of first foundation protocol blocks configured to cause the clinical guidance engine to execute a background task without generating a clinical guidance UI control, and
(b) a plurality of second foundation protocol blocks configured to cause the clinical guidance engine to generate a UI control at the clinical guidance UI.

127. The system of claim 126, wherein the plurality of first foundation protocol blocks comprise medical device interaction protocol blocks, the medical device interaction protocol blocks configured to cause the clinical guidance engine to retrieve respiratory parameters from a communicatively coupled medical device.

128. (canceled)

129. (canceled)

130. (canceled)

131. (canceled)

132. (canceled)

133. (canceled)

134. (canceled)

135. (canceled)

136. The system of claim 1, wherein the encoded clinical guidance protocol comprises a cardiopulmonary resuscitation (CPR) advisory workflow, the CPR advisory workflow comprising:
(a) one or more specified protocol blocks configured to provide chest compression guidance,
(b) one or more specified protocol blocks configured to provide ventilation guidance, and
(c) one or more specified protocol blocks configured to evaluate an ECG rhythm and provide shock delivery guidance based on the ECG rhythm evaluation.

137. (canceled)

138. (canceled)

139. (canceled)

140. (canceled)

141. (canceled)

142. (canceled)

143. (canceled)

144. (canceled)

145. (canceled)

146. (canceled)

147. The system of claim 1, wherein:
each sequencing link connects an output port of a first protocol block with an input port of a second protocol block,
the CGPG GUI is configured to graphically represent each sequencing link, and
the CGPG engine is configured to:
assign a first unique identifier to the output port,
assign a second unique identifier to the input port, and
encode the graphically represented sequencing link as a sequencing instruction in the encoded clinical guidance protocol using the first and the second unique identifiers.

148-219. (canceled)
